(19) United States
(12) Patent Application Publication
(10) Pub. No.: US 2002/0012921 A1
(43) Pub. Date: Jan. 31, 2002

(54) Identification of genetic components of drug response

(76) Inventor: Vincent P. Stanton JR., Belmont, MA (US)

Correspondence Address: ANITA L. MEIKLEJOHN, PH.D., Fish & Richardson P.C., 225 Franklin Street, Boston, MA, 02110-2804, US

(21) Appl. No.: 09/767,892

(22) Filed: Jan. 22, 2001

Related U.S. Application Data
(60) Provisional application No. 60/177,522, filed on Jan. 21, 2000.

(51) Int. Cl. C12Q 1/68; C12Q 1/00

(52) U.S. Cl. 435/6; 435/4

(57) ABSTRACT

The present invention is concerned generally with the field of identifying an appropriate treatment regimen for a disease based upon genotype in mammals, particularly in humans. It is further concerned with the genetic basis of inter-patient variation in response to therapy, including drug therapy. Specifically, this invention describes the identification of gene sequence variances useful in the field of therapeutics for optimizing efficacy and safety of drug therapy. These variances may be useful during the drug development process and in guiding the optimal use of already approved compounds. DNA sequence variances in candidate genes (i.e., genes that may plausibly affect the action of a drug) are tested in clinical trials, leading to the establishment of diagnostic tests useful for improving the development of new pharmaceutical products and/or the more effective use of existing pharmaceutical products. Methods for identifying genetic variances and determining their utility in the selection of optimal therapy for specific patients are also described. In general, the invention relates to methods for identifying patient population subsets that respond to drug therapy with either therapeutic benefit or side effects (i.e., symptomatology prompting concern about safety or other unwanted signs or symptoms).

### TECHNICAL FIELD

This application concerns the field of mammalian therapeutics and the selection of therapeutic regimens utilizing host genetic information, including gene sequence variances within the human genome in human populations. The application further concerns methods for identification of DNA sequence variations likely to affect treatment response, including both in vitro and in vivo approaches.

### BACKGROUND

The information provided below is not admitted to be prior art to the present invention, but is provided solely to assist the understanding of the reader.

Many drugs or other treatments are known to have highly variable safety and efficacy in different individuals. A consequence of such variability is that a given drug or other treatment may be effective in one individual, and ineffective or not well-tolerated in another individual. Thus, administration of such a drug to an individual in whom the drug would be ineffective would result in wasted cost and time during which the patient's condition may significantly worsen. Also, administration of a drug to an individual in whom the drug would not be tolerated could result in a direct worsening of the patient's condition and could even result in the patient's death.

For some drugs, over 90% of the measurable variation in selected pharmacokinetic parameters has been shown to be heritable. For a limited number of drugs, DNA sequence variances have been identified in specific genes that are involved in drug action or metabolism, and these variances have been shown to account for the variable efficacy or safety of the drugs in different individuals. As the sequence of the human genome is completed, and as additional human gene sequence variances are identified, the power of genetic methods for predicting drug response will further increase.

Medical management of human diseases often present unique medical challenges to clinicians, patients, and caregivers. Many diseases progress and the clinical diagnosis may include more than one disorder, dysfunction, or condition. Further, the efficacy of available treatments may be limited and there may be serious, mostly unpredictable, side effects associated with some drugs. The progressive nature of many diseases makes the passage of time a crucial issue in the treatment process. Specifically, selection of optimal treatment for optimal therpaeutic management may be complicated by the fact that it often takes weeks or months to determine if a given therapy is producing a measurable benefit. Thus the current empirical approach to prescribing pharmacotherapy, in which each course of treatment for a given patient is a small experiment, is unsatisfactory from both a medical and economic perspective. Even when an effective treatment is ultimately identified, it often follows a period of ineffective or suboptimal treatment. A method that would help caregivers predict which patients will exhibit beneficial therapeutic responses to a specific medication would provide both medical and economic benefits. As healthcare becomes increasingly costly, the ability to rationally allocate healthcare expenditures, and in particular pharmacy resources, also becomes increasingly important.

### SUMMARY

The present invention is concerned generally with the field of identifying an appropriate treatment regimen for a disease based upon genotype in mammals, particularly in humans. It is further concerned with the genetic basis of inter-patient variation in response to therapy, including drug therapy. Specifically, this invention describes the identification of gene sequence variances useful in the field of therapeutics for optimizing efficacy and safety of drug therapy. These variances may be useful during the drug development process and in guiding the optimal use of already approved compounds. DNA sequence variances in candidate genes (i.e., genes that may plausibly affect the action of a drug) are tested in clinical trials, leading to the establishment of diagnostic tests useful for improving the development of new pharmaceutical products and/or the more effective use of existing pharmaceutical products. Methods for identifying genetic variances and determining their utility in the selection of optimal therapy for specific patients are also described. In general, the invention relates to methods for identifying patient population subsets that respond to drug therapy with either therapeutic benefit or side effects (i.e., symptomatology prompting concern about safety or other unwanted signs or symptoms).

The inventors have determined that the identification of gene sequence variances in genes that may be involved in drug action are useful for determining whether genetic variances account for variable drug efficacy and safety and for determining whether a given drug or other therapy may be safe and effective in an individual patient. Provided in this invention are identifications of genes and sequence variances which can be useful in connection with predicting differences in response to treatment and selection of appropriate treatment of a disease or condition. A target gene and variances are useful, for example, in pharmacogenetic association studies and diagnostic tests to improve the use of certain drugs or other therapies including, but not limited to, the drug classes and specific drugs identified in the 1999 Physicians' Desk Reference (53rd edition), Medical Economics Data, 1998, the 1995 United States Pharmacopeia XXIII National Formulary XVIII, Interpharm Press, 1994, Examples 5-18 or other sources as described below.

The terms “disease” or “condition” are commonly recognized in the art and designate the presence of signs and/or symptoms in an individual or patient that are generally recognized as abnormal. Diseases or conditions may be diagnosed and categorized based on pathological changes. Signs may include any objective evidence of a disease such as changes that are evident by physical examination of a patient or the results of diagnostic tests which may include, among others, laboratory tests to determine the presence of DNA sequence variances or variant forms of certain genes in a patient. Symptoms are subjective evidence of disease or a patients condition, i.e., the patients perception of an abnormal condition that differs from normal function, sensation, or appearance, which may include, without limitations, physical disabilities, morbidity, pain, and other changes from the normal condition experienced by an individual. Various diseases or conditions include, but are not limited to; those categorized in standard textbooks of medicine including, without limitation, textbooks of nutrition, allopathic, homeopathic, and osteopathic medicine. In certain aspects of this invention, the disease or condition is selected from the group consisting of the diseases or conditions identified herein and the types of diseases listed in standard texts such as Harrison's Principles of Internal Medicine (14th Ed) by Anthony S. Fauci, Eugene Braunwald, Kurt J. Isselbacher, et al. (Editors), McGraw Hill, 1997, or Robbins Pathologic Basis of Disease (6th edition) by Ramzi S. Cotran, Vinay Kumar, Tucker Collins & Stanley L. Robbins, W B Saunders Co., 1998, or the Diagnostic and Statistical Manual of Mental Disorders: DSM-IV (4th edition), American Psychiatric Press, 1994, or other texts described below.

In connection with the methods of this invention, unless otherwise indicated, the term “suffering from a disease or condition” means that a person is either presently subject to the signs and symptoms, or is more likely to develop such signs and symptoms than a normal person in the population. Thus, for example, a person suffering from a condition can include a developing fetus, a person subject to a treatment or environmental condition which enhances the likelihood of developing the signs or symptoms of a condition, or a person who is being given or will be given a treatment which increase the likelihood of the person developing a particular condition. For example, tardive dyskinesia is associated with long-term use of anti-psychotics, dyskinesias, paranoid ideation, psychotic episodes and depression have been associated with use of L-dopa in Parkinson's disease; (and dizziness, diplopia, ataxia, sedation, impaired mentation, weight gain, and other undesired effects have been described for various anticonvulsant therapies). Thus, methods of the present invention which relate to treatments of patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

The term “therapy” refers to a process that is intended to produce a beneficial change in the condition of a mammal, e.g., a human, often referred to as a patient. A beneficial change can, for example, include one or more of: restoration of function, reduction of symptoms, limitation or retardation of progression of a disease, disorder, or condition or prevention, limitation or retardation of deterioration of a patient's condition, disease or disorder. Such therapy can involve, for example, nutritional modifications, administration of radiation, administration of a drug, behavioral modifications, and combinations of these, among others.

The term “drug” as used herein refers to a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, lipoproteins, and modifications and combinations thereof. A biological product is preferably a monoclonal or polyclonal antibody or fragment thereof such as a variable chain fragment; cells; or an agent or product arising from recombinant technology, such as, without limitation, a recombinant protein, recombinant vaccine, or DNA construct developed for therapeutic, e.g., human therapeutic, use. The term “drug” may include, without limitation, compounds that are approved for sale as pharmaceutical products by government regulatory agencies (e.g., U.S. Food and Drug Administration (USFDA or FDA), European Medicines Evaluation Agency (EMEA), and a world regulatory body governing the International Conference of Harmonization (ICH) rules and guidelines), compounds that do not require approval by government regulatory agencies, food additives or supplements including compounds commonly characterized as vitamins, natural products, and completely or incompletely characterized mixtures of chemical entities including natural compounds or purified or partially purified natural products. The term “drug” as used herein is synonymous with the terms “medicine”, “pharmaceutical product”, or “product”. Most preferably the drug is approved by a government agency for treatment of a specific disease or condition.

A “low molecular weight compound” has a molecular weight <5,000 Da, more preferably <2500 Da, still more preferably <1000 Da, and most preferably <700 Da.

Those familiar with drug use in medical practice will recognize that regulatory approval for drug use is commonly limited to approved indications, such as to those patients afflicted with a disease or condition for which the drug has been shown to be likely to produce a beneficial effect in a controlled clinical trial. Unfortunately, it has generally not been possible with current knowledge to predict which patients will have a beneficial response, with the exception of certain diseases such as bacterial infections where suitable laboratory methods have been developed. Likewise, it has generally not been possible to determine in advance whether a drug will be safe in a given patient. Regulatory approval for the use of most drugs is limited to the treatment of selected diseases and conditions. The descriptions of approved drug usage, including the suggested diagnostic studies or monitoring studies, and the allowable parameters of such studies, are commonly described in the “label” or “insert” which is distributed with the drug. Such labels or inserts are preferably required by government agencies as a condition for marketing the drug and are listed in common references such as the Physicians Desk Reference (PDR). These and other limitations or considerations on the use of a drug are also found in medical journals, publications such as pharmacology, pharmacy or medical textbooks including, without limitation, textbooks of nutrition, allopathic, homeopathic, and osteopathic medicine.

Many widely used drugs are effective in a minority of patients receiving the drug, particularly when one controls for the placebo effect. For example, the PDR shows that about 45% of patients receiving Cognex (tacrine hydrochloride) for Alzheimer's disease show no change or minimal worsening of their disease, as do about 68% of controls (including about 5% of controls who were much worse). About 58% of Alzheimer's patients receiving Cognex were minimally improved, compared to about 33% of controls, while about 2% of patients receiving Cognex were much improved compared to about 1% of controls. Thus a tiny fraction of patients had a significant benefit. Response to treatments for amyotrophic lateral sclerosis are likewise minimal.

Thus, in a first aspect, the invention provides a method for selecting a treatment for a patient suffering from a disease or condition by determining whether or not a gene or genes in cells of the patient (in some cases including both normal and disease cells, such as cancer cells) contain at least one sequence variance which is indicative of the effectiveness of the treatment of the disease or condition. Preferably the at least one variance includes a plurality of variances. Preferably the at least one variance, or plurality of variances provides or constitues a haplotype or haplotypes. (In each of the aspects of this invention, at least one variance or a plurality of variances preferably provides one or more haplotypes.) Preferably the joint presence of the plurality of variances is indicative of the potential effectiveness or safety of the treatment in a patient having such plurality of variances. The plurality of variances may each be indicative of the potential effectiveness of the treatment, and the effects of the individual variances may be independent or additive, or the plurality of variances may be indicative of the potential effectiveness if at least 2, 3, 4, or more appear jointly. The plurality of variances may also be combinations of these relationships. The plurality of variances may include variances from one, two, three or more gene loci.

In some cases, the selection of a method of treatment, i.e., a therapeutic regimen, may incorporate selection of one or more from a plurality of medical therapies. Thus, the selection may be the selection of a method or methods which is/are more effective or less effective than certain other therapeutic regimens (with either having varying safety parameters). Likewise or in combination with the preceding selection, the selection may be the selection of a method or methods, which is safer than certain other methods of treatment in the patient.

The selection may involve either positive selection or negative selection or both, meaning that the selection can involve a choice that a particular method would be an appropriate method to use and/or a choice that a particular method would be an inappropriate method to use. Thus, in certain embodiments, the presence of the at least one variance is indicative that the treatment will be effective or otherwise beneficial (or more likely to be beneficial) in the patient. Stating that the treatment will be effective means that the probability of beneficial therapeutic effect is greater than in a person not having the appropriate presence or absence of particular variances. In other embodiments, the presence of the at least one variance is indicative that the treatment will be ineffective or contra-indicated for the patient. For example, a treatment may be contra-indicated if the treatment results, or is more likely to result, in undesirable side effects, or an excessive level of undesirable side effects. A determination of what constitutes excessive side-effects will vary, for example, depending on the disease or condition being treated, the availability of alternatives, the expected or experienced efficacy of the treatment, and the tolerance of the patient. As for an effective treatment, this means that it is more likely that desired effect will result from the treatment administration in a patient with a particular variance or variances than in a patient who has a different variance or variances. Also in preferred embodiments, the presence of the at least one variance is indicative that the treatment is both effective and unlikely to result in undesirable effects or outcomes, or vice versa (is likely to have undesirable side effects but unlikely to produce desired therapeutic effects).

In reference to response to a treatment, the term “tolerance” refers to the ability of a patient to accept a treatment, based, e.g., on deleterious effects and/or effects on lifestyle. Frequently, the term principally concerns the patients perceived magnitude of deleterious effects such as nausea, weakness, dizziness, and diarrhea, among others. Such experienced effects can, for example, be due to general or cell-specific toxicity, activity on non-target cells, cross-reactivity on non-target cellular constituents (non-mechanism based), and/or side effects of activity on the target cellular substituents (mechanism based), or the cause of toxicity may not be understood. In any of these circumstances one may identify an association between the undesirable effects and variances in specific genes.

Adverse responses to drugs constitute a major medical problem, as shown in two recent meta-analyses (Lazarou, J. et at, Incidence of adverse drug reactions in hospitalized patients: a meta-analysis of prospective studies, JAMA 279:1200-1205, 1998; Bonn, Adverse drug reactions remain a major cause of death, Lancet 3 51: 1183, 1998). An estimated 2.2 million hospitalized patients in the United Stated had serious adverse drug reactions in 1994, with an estimated 106,000 deaths (Lazarou et al.). To the extent that some of these adverse events are due to genetically encoded biochemical diversity among patients in pathways that effect drug action, the identification of variances that are predictive of such effects will allow for more effective and safer drug use.

In embodiments of this invention, the variance or variant form or forms of a gene is/are associated with a specific response to a drug. The frequency of a specific variance or variant form of the gene may correspond to the frequency of an efficacious response to administration of a drug. Alternatively, the frequency of a specific variance or variant form of the gene may correspond to the frequency of an adverse event resulting from administration of a drug. Alternatively the frequency of a specific variance or variant form of a gene may not correspond closely with the frequency of a beneficial or adverse response, yet the variance may still be useful for identifying a patient subset with high response or toxicity incidence because the variance may account for only a fraction of the patients with high response or toxicity. In such a case the preferred course of action is identification of a second or third or additional variances that permit identification of the patient groups not usefully identified by the first variance. Preferably, the drug will be effective in more than 20% of individuals with one or more specific variances or variant forms of the gene, more preferably in 40% and most preferably in >60%. In other embodiments, the drug will be toxic or create clinically unacceptable side effects in more than 10% of individuals with one or more variances or variant forms of the gene, more preferably in >30%, more preferably in >50%, and most preferably in >70% or in more than 90%.

Also in other embodiments, the method of selecting a treatment includes excluding or eliminating a treatment, where the presence or absence of the at least one variance is indicative that the treatment will be ineffective or contra-indicated, e.g., would result in excessive weight gain. In other preferred embodiments, in cases in which undesirable side-effects may occur or are expected to occur from a particular therapeutic treatment, the selection of a method of treatment can include identifying both a first and second treatment, where the first treatment is effective to treat the disease or condition, and the second treatment reduces a deleterious effect of the first treatment.

The phrase “eliminating a treatment” or “excluding a treatment” refers to removing a possible treatment from consideration, e.g., for use with a particular patient based on the presence or absence of a particular variance(s) in one or more genes in cells of that patient, or to stopping the administration of a treatment.

Usually, the treatment will involve the administration of a compound preferentially active or safe in patients with a form or forms of a gene, where the gene is one identified herein. The administration may involve a combination of compounds. Thus, in preferred embodiments, the method involves identifying such an active compound or combination of compounds, where the compound is less active or is less safe or both when administered to a patient having a different form of the gene.

Also in preferred embodiments, the method of selecting a treatment involves selecting a method of administration of a compound, combination of compounds, or pharmaceutical composition, for example, selecting a suitable dosage level and/or frequency of administration, and/or mode of administration of a compound. The method of administration can be selected to provide better, preferably maximum therapeutic benefit. In this context, “maximum” refers to an approximate local maximum based on the parameters being considered, not an absolute maximum.

Also in this context, a “suitable dosage level” refers to a dosage level that provides a therapeutically reasonable balance between pharmacological effectiveness and deleterious effects. Often this dosage level is related to the peak or average serum levels resulting from administration of a drug at the particular dosage level.

Similarly, a “frequency of administration” refers to how often in a specified time period a treatment is administered, e.g., once, twice, or three times per day, every other day, once per week, etc. For a drug or drugs, the frequency of administration is generally selected to achieve a pharmacologically effective average or peak serum level without excessive deleterious effects (and preferably while still being able to have reasonable patient compliance for self-administered drugs). Thus, it is desirable to maintain the serum level of the drug within a therapeutic window of concentrations for the greatest percentage of time possible without such deleterious effects as would cause a prudent physician to reduce the frequency of administration for a particular dosage level.

A particular gene or genes can be relevant to the treatment of more than one disease or condition, for example, the gene or genes can have a role in the initiation, development, course, treatment, treatment outcomes, or health-related quality of life outcomes of a number of different diseases, disorders, or conditions. Thus, in preferred embodiments, the disease or condition or treatment of the disease or condition is any which involves a gene from the gene list described in U.S. Ser. No. 09/689,506 (filed Oct. 13, 2000), hereby incorporated by reference.

Determining the presence of a particular variance or plurality of variances in a particular gene in a patient can be performed in a variety of ways. In preferred embodiments, the detection of the presence or absence of at least one variance involves amplifying a segment of nucleic acid including at least one of the at least one variances. Preferably a segment of nucleic acid to be amplified is 500 nucleotides or less in length, more preferably 100 nucleotides or less, and most preferably 45 nucleotides or less. Also, preferably the amplified segment or segments includes a plurality of variances, or a plurality of segments of a gene or of a plurality of genes. In other embodiments, e.g., where a haplotype is to be determined, the segment of nucleic acid is at least 500 nucleotides in length, or at least 2 kb in length, or at least 5 kb in length.

In preferred embodiments, determining the presence of a set of variances in a specific gene related to treatment of neurological disease or other related genes, or genes listed in In U.S. patent application Ser. No. 09/689,506, includes a haplotyping test that involves allele specific amplification of a large DNA segment of no greater than 25,000 nucleotides, preferably no greater than 10,000 nucleotides and most preferably no greater than 5,000 nucleotides. Alternatively one allele may be enriched by methods other than amplification prior to determining genotypes at specific variant positions on the enriched allele as a way of determining haplotypes. Preferably the determination of the presence or absence of a haplotype involves determining the sequence of the variant sites by methods such as chain terminating DNA sequencing or minisequencing, or by oligonucleotide hybridization or by mass spectrometry.

The term “genotype” in the context of this invention refers to the alleles present in DNA from a subject or patient, where an allele can be defined by the particular nucleotide(s) present in a nucleic acid sequence at a particular site(s). Often a genotype is the nucleotide(s) present at a single polymorphic site known to vary in the human population.

In preferred embodiments, the detection of the presence or absence of the at least one variance involves contacting a nucleic acid sequence corresponding to one of the genes identified above or a product of such a gene with a probe. The probe is able to distinguish a particular form of the gene or gene product or the presence or a particular variance or variances, e.g., by differential binding or hybridization. Thus, exemplary probes include nucleic acid hybridization probes, peptide nucleic acid probes, nucleotide-containing probes which also contain at least one nucleotide analog, and antibodies, e.g., monoclonal antibodies, and other probes as discussed herein. Those skilled in the art are familiar with the preparation of probes with particular specificities. Those skilled in the art will recognize that a variety of variables can be adjusted to optimize the discrimination between two variant forms of a gene, including changes in salt concentration, temperature, pH and addition of various compounds that affect the differential affinity of GC vs. AT base pairs, such as tetramethyl ammonium chloride. (See Current Protocols in Molecular Biology by F. M. Ausubel, R. Brent, R. E. Kngston, D. D. Moore, J. D. Seidman, K. Struhl, and V. B. Chanda (editors, John Wiley & Sons.)

In other preferred embodiments, determining the presence or absence of the at least one variance involves sequencing at least one nucleic acid sample. The sequencing involves sequencing of a portion or portions of a gene and/or portions of a plurality of genes which includes at least one variance site, and may include a plurality of such sites. Preferably, the portion is 500 nucleotides or less in length, more preferably 100 nucleotides or less, and most preferably 45 nucleotides or less in length. Such sequencing can be carried out by various methods recognized by those skilled in the art, including use of dideoxy termination methods (e.g., using dye-labeled dideoxy nucleotides) and the use of mass spectrometric methods. In addition, mass spectrometric methods may be used to determine the nucleotide present at a variance site. In preferred embodiments in which a plurality of variances is determined, the plurality of variances can constitute a haplotype or collection of haplotypes. Preferably the methods for determining genotypes or haplotypes are designed to be sensitive to all the common genotypes or haplotypes present in the population being studied (for example, a clinical trial population).

The terms “variant form of a gene”, “form of a gene”, or “allele” refer to one specific form of a gene in a population, the specific form differing from other forms of the same gene in the sequence of at least one, and frequently more than one, variant sites within the sequence of the gene. The sequences at these variant sites that differ between different alleles of the gene are termed “gene sequence variances” or “variances” or “variants”. The term “alternative form” refers to an allele that can be distinguished from other alleles by having distinct variances at least one, and frequently more than one, variant sites within the gene sequence. Other terms known in the art to be equivalent include mutation and polymorphism, although mutation is often used to refer to an allele associated with a deleterious phenotype. In preferred aspects of this invention, the variances are selected from the group consisting of the variances listed in the variance tables herein or in a patent or patent application referenced and incorporated by reference in this disclosure. In the methods utilizing variance presence or absence, reference to the presence of a variance or variances means particular variances, i.e., particular nucleotides at particular polymorphic sites, rather than just the presence of any variance in the gene.

Variances occur in the human genome at approximately one in every 500-1,000 bases within the human genome when two alleles are compared. When multiple alleles from unrelated individuals are compared the density of variant sites increases as different individuals, when compared to a reference sequence, will often have sequence variances at different sites. At most variant sites there are only two alternative nucleotides involving the substitution of one base for another or the insertion/deletion of one or more nucleotides. Within a gene there may be several variant sites. Variant forms of the gene or alternative alleles can be distinguished by the presence of alternative variances at a single variant site, or a combination of several different variances at different sites (haplotypes).

It is estimated that there are 3,300,000,000 bases in the sequence of a single haploid human genome. All human cells except germ cells are normally diploid. Each gene in the genome may span 100-10,000,000 bases of DNA sequence or 100-20,000 bases of mRNA. It is estimated that there are between 60,000 and 150,000 genes in the human genome. The “identification” of genetic variances or variant forms of a gene involves the discovery of variances that are present in a population. The identification of variances is required for development of a diagnostic test to determine whether a patient has a variant form of a gene that is known to be associated with a disease, condition, or predisposition or with the efficacy or safety of the drug. Identification of previously undiscovered genetic variances is distinct from the process of “determining” the status of known variances by a diagnostic test (often referred to as genotyping). The present invention provides exemplary variances in genes listed in the gene tables, as well as methods for discovering additional variances in those genes and a comprehensive written description of such additional possible variances. Also described are methods for DNA diagnostic tests to determine the DNA sequence at a particular variant site or sites.

The process of “identifying” or discovering new variances involves comparing the sequence of at least two alleles of a gene, more preferably at least 10 alleles and most preferably at least 50 alleles (keeping in mind that each somatic cell has two alleles). The analysis of large numbers of individuals to discover variances in the gene sequence between individuals in a population will result in detection of a greater fraction of all the variances in the population. Preferably the process of identifying reveals whether there is a variance within the gene; more preferably identifying reveals the location of the variance within the gene; more preferably identifying provides knowledge of the sequence of the nucleic acid sequence of the variance, and most preferably identifying provides knowledge of the combination of different variances that comprise specific variant forms of the gene (referred to as alleles). In identifying new variances it is often useful to screen different population groups based on racial, ethnic, gender, and/or geographic origin because particular variances may differ in frequency between such groups. It may also be useful to screen DNA from individuals with a particular disease or condition of interest because they may have a higher frequency of certain variances than the general population.

The process of genotyping involves using diagnostic tests for specific variances that have already been identified. It will be apparent that such diagnostic tests can only be performed after variances and variant forms of the gene have been identified. Identification of new variances can be accomplished by a variety of methods, alone or in combination, including, for example, DNA sequencing, SSCP, heteroduplex analysis, denaturing gradient gel electrophoresis (DGGE), heteroduplex cleavage (either enzymatic as with T4 Endonuclease 7, or chemical as with osmium tetroxide and hydroxylamine), computational methods (described herein), and other methods described herein as well as others known to those skilled in the art. (See, for example: Cotton, R. G. H., Slowly but surely towards better scanning for mutations, Trends in Genetics 13(2): 43-6, 1997 or Current Protocols in Human Genetics by N. C. Dracoli, J. L. Haines, B. R. Korf, D. T. Moir, C. C. Morton, C. E. Seidman, D. R. Smith, and A. Boyle (editors), John Wiley & Sons.)

In the context of this invention, the term “analyzing a sequence” refers to determining at least some sequence information about the sequence, e.g., determining the nucleotides present at a particular site or sites in the sequence, particularly sites that are known to vary in a population, or determining the base sequence of all or of a portion of the particular sequence.

In the context of this invention, the term “haplotype” refers to a cis arrangement of two or more polymorphic nucleotides, i.e., variances, on a particular chromosome, e.g., in a particular gene. The haplotype preserves information about the phase of the polymorphic nucleotides—that is, which set of variances were inherited from one parent, and which from the other. A genotyping test does not provide information about phase. For example, an individual heterozygous at nucleotide 25 of a gene (both A and C are present) and also at nucleotide 100 (both G and T are present) could have haplotypes 25A-100G and 25C-100T, or alternatively 25A-100T and 25C-100G. Only a haplotyping test can discriminate these two cases definitively.

The terms “variances”, “variants” and “polymorphisms”, as used herein, may also refer to a set of variances, haplotypes or a mixture of the two, unless otherwise indicated. Further, the term variance, variant or polymorphism (singular), as used herein, also encompasses a haplotype unless otherwise indicated. This usage is intended to minimize the need for cumbersome phrases such as: “. . . measure correlation between drug response and a variance, variances, haplotype, haplotypes or a combination of variances and haplotypes . . .”, throughout the application. Instead, the italicized text in the foregoing sentence can be represented by the word “variance”, “variant” or “polymorphism”. Similarly, the term “genotype”, as used herein, means a procedure for determining the status of one or more variances in a gene, including a set of variances comprising a haplotype. Thus phrases such as “. . . genotype a patient . . . ” refer to determining the status of one or more variances, including a set of variances for which phase is known (i.e. a haplotype).

In preferred embodiments of this invention, the frequency of the variance or variant form of the gene in a population is known. Measures of frequency known in the art include “allele frequency”, namely the fraction of genes in a population that have one specific variance or set of variances. The allele frequencies for any gene should sum to 1. Another measure of frequency known in the art is the “heterozygote frequency” namely, the fraction of individuals in a population who carry two alleles, or two forms of a particular variance or variant form of a gene, one inherited from each parent. Alternatively, the number of individuals who are homozygous for a particular form of a gene may be a useful measure. The relationship between allele frequency, heterozygote frequency, and homozygote frequency is described for many genes by the Hardy-Weinberg equation, which provides the relationship between allele frequency, heterozygote frequency and homozygote frequency in a freely breeding population at equilibrium. Most human variances are substantially in Hardy-Weinberg equilibrium. In a preferred aspect of this invention, the allele frequency, heterozygote frequency, and homozygote frequencies are determined experimentally. Preferably a variance has an allele frequency of at least 0.01, more preferably at least 0.05, still more preferably at least 0.10. However, the allele may have a frequency as low as 0.001 if the associated phenotype is, for example, a rare form of toxic reaction to a treatment or drug. Beneficial responses may also be rare.

In this regard, “population” refers to a defined group of individuals or a group of individuals with a particular disease or condition or individuals that may be treated with a specific drug identified by, but not limited to geographic, ethnic, race, gender, and/or cultural indices. In most cases a population will preferably encompass at least ten thousand, one hundred thousand, one million, ten million, or more individuals, with the larger numbers being more preferable. In preferred embodiments of this invention, the population refers to individuals with a specific disease or condition that may be treated with a specific drug. In embodiments of this invention, the allele frequency, heterozygote frequency, or homozygote frequency of a specific variance or variant form of a gene is known. In preferred embodiments of this invention, the frequency of one or more variances that may predict response to a treatment is determined in one or more populations using a diagnostic test.

It should be emphasized that it is currently not generally practical to study an entire population to establish the association between a specific disease or condition or response to a treatment and a specific variance or variant form of a gene. Such studies are preferably performed in controlled clinical trials using a limited number of patients that are considered to be representative of the population with the disease. Since drug development programs are generally targeted at the largest possible population, the study population will generally consist of men and women, as well as members of various racial and ethnic groups, depending on where the clinical trial is being performed. This is important to establish the efficacy of the treatment in all segments of the population.

In the context of this invention, the term “probe” refers to a molecule that detectably distinguishes between target molecules differing in structure. Detection can be accomplished in a variety of different ways depending on the type of probe used and the type of target molecule. Thus, for example, detection may be based on discrimination of activity levels of the target molecule, but preferably is based on detection of specific binding. Examples of such specific binding include antibody binding and nucleic acid probe hybridization. Thus, for example, probes can include enzyme substrates, antibodies and antibody fragments, and nucleic acid hybridization probes. Thus, in preferred embodiments, the detection of the presence or absence of the at least one variance involves contacting a nucleic acid sequence which includes a variance site with a probe, preferably a nucleic acid probe, where the probe preferentially hybridizes with a form of the nucleic acid sequence containing a complementary base at the variance site as compared to hybridization to a form of the nucleic acid sequence having a non-complementary base at the variance site, where the hybridization is carried out under selective hybridization conditions. Such a nucleic acid hybridization probe may span two or more variance sites. Unless otherwise specified, a nucleic acid probe can include one or more nucleic acid analogs, labels or other substituents or moieties so long as the base-pairing function is retained.

As is generally understood, administration of a particular treatment, e.g., administration of a therapeutic compound or combination of compounds, is chosen depending on the disease or condition that is to be treated. Thus, in certain preferred embodiments, the disease or condition is one for which administration of a treatment is expected to provide a therapeutic benefit; in certain embodiments, the compound is a compound identified as described in a drug table in U.S. patent Ser. No. 09/689,506.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the presence of one or more sequence variances or alleles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

Effectiveness is measured in a particular population. In conventional drug development the population is generally every subject who meets the enrollment criteria (i.e. has the particular form of the disease or condition being treated). It is an aspect of the present invention that segmentation of a study population by genetic criteria can provide the basis for identifying a subpopulation in which a drug is effective against the disease or condition being treated.

The term “deleterious effects” refers to physical effects in a patient caused by administration of a treatment which are regarded as medically undesirable. Thus, for example, deleterious effects can include a wide spectrum of toxic effects injurious to health such as death of normally functioning cells when only death of diseased cells is desired, nausea, fever, inability to retain food, dehydration, damage to critical organs such as arrythmias, renal tubular necrosis, fatty liver, or pulmonary fibrosis leading to coronary, renal, hepatic, or pulmonary insufficiency among many others. In this regard, the term “contra-indicated” means that a treatment results in deleterious effects such that a prudent medical doctor treating such a patient would regard the treatment as unsuitable for administration. Major factors in such a determination can include, for example, availability and relative advantages of alternative treatments, consequences of non-treatment, and permanency of deleterious effects of the treatment.

It is recognized that many treatment methods, e.g., administration of certain compounds or combinations of compounds, may produce side-effects or other deleterious effects in patients. Such effects can limit or even preclude use of the treatment method in particular patients, or may even result in irreversible injury, dysfunction, or death of the patient. Thus, in certain embodiments, the variance information is used to select both a first method of treatment and a second method of treatment. Usually the first treatment is a primary treatment that provides a physiological effect directed against the disease or condition or its symptoms. The second method is directed to reducing or eliminating one or more deleterious effects of the first treatment, e.g., to reduce a general toxicity or to reduce a side effect of the primary treatment. Thus, for example, the second method can be used to allow use of a greater dose or duration of the first treatment, or to allow use of the first treatment in patients for whom the first treatment would not be tolerated or would be contra-indicated in the absence of a second method to reduce deleterious effects or to potentiate the effectiveness of the first treatment.

In a related aspect, the invention concerns a method for providing a correlation or other statistical test of relationship between a patient genotype and effectiveness of a treatment, by determining the presence or absence of a particular known variance or variances in cells of a patient for a gene gene in U.S. patent application Ser. No. 09/689,506, or other gene related to neurological disease, and providing a result indicating the expected effectiveness of a treatment for a disease or condition. The result may be formulated by comparing the genotype of the patient with a list of variances indicative of the effectiveness of a treatment, e.g., administration of a drug described herein. The determination may be by methods as described herein or other methods known to those skilled in the art.

In a related aspect, the invention provides a method for selecting a method of treatment for a patient suffering from a disease or condition by comparing at least one variance in at least one gene in the patient, with a list of variances in the gene from U.S. patent application Ser. No. 09/689,506, or other gene related to neurological disease, which are indicative of the effectiveness of at least one method of treatment. Preferably the comparison involves a plurality of variances or a haplotype indicative of the effectiveness of at least one method of treatment. Also, preferably the list of variances includes a plurality of variances.

Similar to the above aspect, in preferred embodiments the at least one method of treatment involves the administration of a compound effective in at least some patients with a disease or condition; the presence or absence of the at least one variance is indicative that the treatment will be effective in the patient; and/or the presence or absence of the at least one variance is indicative that the treatment will be ineffective or contra-indicated in the patient; and/or the treatment is a first treatment and the presence or absence of the at least one variance is indicative that a second treatment will be beneficial to reduce a deleterious effect of or potentiate the effectiveness of the first treatment; and/or the at least one treatment is a plurality of methods of treatment. For a plurality of treatments, preferably the selecting involves determining whether any of the methods of treatment will be more effective than at least one other of the plurality of methods of treatment. Yet other embodiments are provided as described for the preceding aspect in connection with methods of treatment using administration of a compound; treatment of various diseases, and variances in particular genes.

In the context of variance information in the methods of this invention, the term “list” refers to one or more, preferably at least 2, 3, 4, 5, 7, or 10 variances that have been identified for a gene of potential importance in accounting for inter-individual variation in treatment response. Preferably there is a plurality of variances for the gene, preferably a plurality of variances for the particular gene. Preferably, the list is recorded in written or electronic form. For example, identified variances of identified genes are recorded for some of the genes in U.S. patent application Ser. No. 09/689,506; additional variances for genes are provided in Table 1 of Stanton et al., U.S. application Ser. No. 09/300,747 or related CIP application, and additional gene variance identification tables are provided in a form which allows comparison with other variance information. The possible additional variances in the identified genes are provided in Table 3 in Stanton et al., U.S. application Ser. No. 09/300,747.

In addition to the basic method of treatment, often the mode of administration of a given compound as a treatment for a disease or condition in a patient is significant in determining the course and/or outcome of the treatment for the patient. Thus, the invention also provides a method for selecting a method of administration of a compound to a patient suffering from a disease or condition, by determining the presence or absence of at least one variance in cells of the patient in at least one identified gene in U.S. patent application Ser. No. 09/689,506, where such presence or absence is indicative of an appropriate method of administration of the compound. Preferably, the selection of a method of treatment (a treatment regimen) involves selecting a dosage level or frequency of administration or route of administration of the compound or combinations of those parameters. In preferred embodiments, two or more compounds are to be administered, and the selecting involves selecting a method of administration for one, two, or more than two of the compounds, jointly, concurrently, or separately. As understood by those skilled in the art, such plurality of compounds may be used in combination therapy, and thus may be formulated in a single drug, or may be separate drugs administered concurrently, serially, or separately. Other embodiments are as indicated above for selection of second treatment methods, methods of identifying variances, and methods of treatment as described for aspects above.

In another aspect, the invention provides a method for selecting a patient for administration of a method of treatment for a disease or condition, or of selecting a patient for a method of administration of a treatment, by comparing the presence or absence of at least one variance in a gene as identified above in cells of a patient, with a list of variances in the gene, where the presence or absence of the at least one variance is indicative that the treatment or method of administration will be effective in the patient. If the at least one variance is present in the patient's cells, then the patient is selected for administration of the treatment.

In preferred embodiments, the disease or the method of treatment is as described in aspects above, specifically including, for example, those described for selecting a method of treatment.

In another aspect, the invention provides a method for identifying a subset of patients with enhanced or diminished response or tolerance to a treatment method or a method of administration of a treatment where the treatment is for a disease or condition in the patient. The method involves correlating one or more variances in one or more genes as identified in aspects above in a plurality of patients with response to a treatment or a method of administration of a treatment. The correlation may be performed by determining the one or more variances in the one or more genes in the plurality of patients and correlating the presence or absence of each of the variances (alone or in various combinations) with the patient's response to treatment. The variances may be previously known to exist or may also be determined in the present method or combinations of prior information and newly determined information may be used. The enhanced or diminished response should be statistically significant, preferably such that p=0.10 or less, more preferably 0.05 or less, and most preferably 0.02 or less. A positive correlation between the presence of one or more variances and an enhanced response to treatment is indicative that the treatment is particularly effective in the group of patients having those variances. A positive correlation of the presence of the one or more variances with a diminished response to the treatment is indicative that the treatment will be less effective in the group of patients having those variances. Such information is useful, for example, for selecting or de-selecting patients for a particular treatment or method of administration of a treatment, or for demonstrating that a group of patients exists for which the treatment or method of treatment would be particularly beneficial or contra-indicated. Such demonstration can be beneficial, for example, for obtaining government regulatory approval for a new drug or a new use of a drug

In preferred embodiments, the variances are in at least one of the identified genes listed in U.S. patent application Ser. No. 09/689,506, or are particular variances described herein. Also, preferred embodiments include drugs, treatments, variance identification or determination, determination of effectiveness, and/or diseases as described for aspects above or otherwise described herein.

In preferred embodiments, the correlation of patient responses to therapy according to patient genotype is carried out in a clinical trial, e.g., as described herein according to any of the variations described. Detailed description of methods for associating variances with clinical outcomes using clinical trials are provided below. Further, in preferred embodiments the correlation of pharmacological effect (positive or negative) to treatment response according to genotype or haplotype in such a clinical trial is part of a regulatory submission to a government agency leading to approval of the drug. Most preferably the compound or compounds would not be approvable in the absence of the genetic information allowing identification of an optimal responder population.

As indicated above, in aspects of this invention involving selection of a patient for a treatment, selection of a method or mode of administration of a treatment, and selection of a patient for a treatment or a method of treatment, the selection may be positive selection or negative selection. Thus, the methods can include eliminating or excluding a treatment for a patient, eliminating or excluding a method or mode of administration of a treatment to a patient, or elimination of a patient for a treatment or method of treatment.

Also, in methods involving identification and/or comparison of variances present in a gene of a patient, the methods can involve such identification or comparison for a plurality of genes. Preferably, the genes are functionally related to the same disease or condition, or to the aspect of disease pathophysiology that is being subjected to pharmacological manipulation by the treatment (e.g., a drug), or to the activation or inactivation or elimination of the drug, and more preferably the genes are involved in the same biochemical process or pathway.

In another aspect, the invention provides a method for identifying the forms of a gene in an individual, where the gene is one specified as for aspects above, by determining the presence or absence of at least one variance in the gene. In preferred embodiments, the at least one variance includes at least one variance selected from the group of variances identified in variance tables herein. Preferably, the presence or absence of the at least one variance is indicative of the effectiveness of a therapeutic treatment in a patient suffering from a disease or condition and having cells containing the at least one variance.

The presence or absence of the variances can be determined in any of a variety of ways as recognized by those skilled in the art. For example, the nucleotide sequence of at least one nucleic acid sequence which includes at least one variance site (or a complementary sequence) can be determined, such as by chain termination methods, hybridization methods or by mass spectrometric methods. Likewise, in preferred embodiments, the determining involves contacting a nucleic acid sequence or a gene product of one of one of the genes with a probe which specifically identifies the presence or absence of a form of the gene. For example, a probe, e.g., a nucleic acid probe, can be used which specifically binds, e.g., hybridizes, to a nucleic acid sequence corresponding to a portion of the gene and which includes at least one variance site under selective binding conditions. As described for other aspects, determining the presence or absence of at least two variances and their relationship on the two gene copies present in a patient can constitute determining a haplotype or haplotypes. In this and other aspects involving mass spectrometry, the method can involve detection of the mass of a fragment or fragments and can further involve inferring the genotype (e.g., the specific variance at a site) from the masses determined.

Other preferred embodiments involve variances related to types of treatment, drug responses, diseases, nucleic acid sequences, and other items related to variances and variance determination as described for aspects above.

In yet another aspect, the invention provides a pharmaceutical composition which includes a compound which has a differential effect in patients having at least one copy, or alternatively, two copies of a form of a gene as identified for aspects above and a pharmaceutically acceptable carrier, excipient, or diluent. The composition is adapted to be preferentially effective to treat a patient with cells containing the one, two, or more copies of the form of the gene.

In preferred embodiments of aspects involving pharmaceutical compositions, active compounds, or drugs, the material is subject to a regulatory limitation or restriction on approved uses or indications, e.g., by the U.S. Food and Drug Administration (FDA), recommending use in or limiting approved use of the composition to patients having at least one copy of the particular form of the gene which contains at least one variance. Alternatively, the composition is subject to a regulatory limitation or restriction or recommendation on approved uses indicating that the composition is not approved for use or should not be used in patients having at least one copy of a form of the gene including at least one variance. Also in preferred embodiments, the composition is packaged, and the packaging includes a label or insert indicating or suggesting beneficial therapeutic approved use of the composition in patients having one or two copies of a form of the gene including at least one variance. Alternatively, the label or insert recommends or limits approved use of the composition to patients having zero or one or two copies of a form of the gene including at least one variance. The latter embodiment would be likely where the presence of the at least one variance in one or two copies in cells of a patient means that the composition would be ineffective or deleterious to the patient. Also in preferred embodiments, the composition is indicated for use in treatment of a disease or condition which is one of those identified for aspects above. Also in preferred embodiments, the at least one variance includes at least one variance from those identified herein.

The term “packaged” means that the drug, compound, or composition is prepared in a manner suitable for distribution or shipping with a box, vial, pouch, bubble pack, or other protective container, which may also be used in combination. The packaging may have printing on it and/or printed material may be included in the packaging.

In preferred embodiments, the drug is selected from the drug classes or specific exemplary drugs identified in an example, in a table herein, and is subject to a regulatory limitation or suggestion or warning as described above that limits or suggests limiting approved use to patients having specific variances or variant forms of a gene identified in Examples or in the gene list provided below in order to achieve maximal benefit and avoid toxicity or other deleterious effect.

A pharmaceutical composition can be adapted to be preferentially effective in a variety of ways. In some cases, an active compound is selected which was not previously known to be differentially active, or which was not previously recognized as a potential therapeutic compound. In some cases, the concentration of an active compound which has differential activity can be adjusted such that the composition is appropriate for administration to a patient with the specified variances. For example, the presence of a specified variance may allow or require the administration of a much larger dose, which would not be practical with a previously utilized composition. Conversely, a patient may require a much lower dose, such that administration of such a dose with a prior composition would be impractical or inaccurate. Thus, the composition may be prepared in a higher or lower unit dose form, or prepared in a higher or lower concentration of the active compound or compounds. In yet other cases, the composition can include additional compounds needed to enable administration of a particular active compound in a patient with the specified variances, which was not in previous compositions, e.g., because the majority of patients did not require or benefit from the added component, or would be adversely affected by the added component(s).

The term “differential” or “differentially” generally refers to a statistically significant different level in the specified property or effect. Preferably, the difference is also functionally significant. Thus, “differential binding or hybridization” is sufficient difference in binding or hybridization to allow discrimination using an appropriate detection technique. Likewise, “differential effect” or “differentially active” in connection with a therapeutic treatment or drug refers to a difference in the level of the effect or activity which is distinguishable using relevant parameters and techniques for measuring the effect or activity being considered. Preferably the difference in effect or activity is also sufficient to be clinically significant, such that a corresponding difference in the course of treatment or treatment outcome would be expected, at least on a statistical basis.

Also usefully provided in the present invention are probes which specifically recognize a nucleic acid sequence corresponding to a variance or variances in a gene as identified in aspects above or a product expressed from the gene, and are able to distinguish a variant form of the sequence or gene or gene product from one or more other variant forms of that sequence, gene, or gene product under selective conditions. Those skilled in the art recognize and understand the identification or determination of selective conditions for particular probes or types of probes. An exemplary type of probe is a nucleic acid hybridization probe, which will selectively bind under selective binding conditions to a nucleic acid sequence or a gene product corresponding to one of the genes identified for aspects above. Another type of probe is a peptide or protein, e.g., an antibody or antibody fragment which specifically or preferentially binds to a polypeptide expressed from a particular form of a gene as characterized by the presence or absence of at least one variance. Thus, in another aspect, the invention concerns such probes. In the context of this invention, a “probe” is a molecule, commonly a nucleic acid, though also potentially a protein, carbohydrate, polymer, or small molecule, that is capable of binding to one variance or variant form of the gene to a greater extent than to a form of the gene having a different base at one or more variance sites, such that the presence of the variance or variant form of the gene can be determined. Preferably the probe distinguishes at least one variance identified in the Examples or in Tables 1 or 3 of Stanton et al., U.S. application Ser. No. 09/300,747.

In preferred embodiments, the probe is a nucleic acid probe at least 15, preferably at least 17 nucleotides in length, more preferably at least 20 or 22 or 25, preferably 500 or fewer nucleotides in length, more preferably 200 or 100 or fewer, still more preferably 50 or fewer, and most preferably 30 or fewer. In preferred embodiments, the probe has a length in a range between from any one of the above lengths to any other of the above lengths (including endpoints). The probe specifically hybridizes under selective hybridization conditions to a nucleic acid sequence corresponding to a portion of one of the genes identified in connection with above aspects. For certain types of probes, e.g., PNA probes, the probe is often shorter, e.g., at least 6, 7, 8, 10, or 12 nucleotides in length, with the length preferably also being no more than 50, 40, 30, 20, 17, or 15 nucleotides in length. The nucleic acid sequence includes at least one variance site. Also in preferred embodiments, the probe has a detectable label, preferably a fluorescent label. A variety of other detectable labels are known to those skilled in the art. Such a nucleic acid probe can also include one or more nucleic acid analogs.

In preferred embodiments, the probe is an antibody or antibody fragment which specifically binds to a gene product expressed from a form of one of the above genes, where the form of the gene has at least one specific variance with a particular base at the variance site, and preferably a plurality of such variances.

In connection with nucleic acid probe hybridization, the term “specifically hybridizes” indicates that the probe hybridizes to a sufficiently greater degree to the target sequence than to a sequence having a mismatched base at least one variance site to allow distinguishing such hybridization. The term “specifically hybridizes” thus means that the probe hybridizes to the target sequence, and not to non-target sequences, at a level which allows ready identification of probe/target sequence hybridization under selective hybridization conditions. Thus, “selective hybridization conditions” refer to conditions that allow such differential binding. Similarly, the terms “specifically binds” and “selective binding conditions” refer to such differential binding of any type of probe, e.g., antibody probes, and to the conditions which allow such differential binding. Typically hybridization reactions to determine the status of variant sites in patient samples are carried out with two different probes, one specific for each of the (usually two) possible variant nucleotides. The complementary information derived from the two separate hybridization reactions is useful in corroborating the results.

Likewise, the invention provides an isolated, purified or enriched nucleic acid sequence of 15 to 500 nucleotides in length, preferably 15 to 100 nucleotides in length, more preferably 15 to 50 nucleotides in length, and most preferably 15 to 30 nucleotides in length, which has a sequence which corresponds to a portion of one of the genes identified for aspects above. Preferably the lower limit for the preceding ranges is 17, 20, 22, or 25 nucleotides in length. In other embodiments, the nucleic acid sequence is 30 to 300 nucleotides in length, or 45 to 200 nucleotides in length, or 45 to 100 nucleotides in length. The nucleic acid sequence includes at least one variance site. Such sequences can, for example, be amplification products of a sequence which spans or includes a variance site in a gene identified herein. Likewise, such a sequence can be a primer that is able to bind to or extend through a variance site in such a gene. Yet another example is a nucleic acid hybridization probe comprised of such a sequence. In such probes, primers, and amplification products, the nucleotide sequence can contain a sequence or site corresponding to a variance site or sites, for example, a variance site identified herein. Preferably the presence or absence of a particular variant form in the heterozygous or homozygous state is indicative of the effectiveness of a method of treatment in a patient.

In reference to nucleic acid sequences which “correspond” to a gene, the term “correspond” refers to a nucleotide sequence relationship, such that the nucleotide sequence has a nucleotide sequence which is the same as the reference gene or an indicated portion thereof, or has a nucleotide sequence which is exactly complementary in normal Watson-Crick base pairing, or is an RNA equivalent of such a sequence, e.g., an mRNA, or is a cDNA derived from an mRNA of the gene.

In another aspect, the invention provides a method for determining a genotype of an individual in relation to one or more variances in one or more of the genes identified in above aspects by using mass spectrometric determination of a nucleic acid sequence which is a portion of a gene identified for other aspects of this invention or a complementary sequence. Such mass spectrometric methods are known to those skilled in the art. In preferred embodiments, the method involves determining the presence or absence of a variance in a gene; determining the nucleotide sequence of the nucleic acid sequence; the nucleotide sequence is 100 nucleotides or less in length, preferably 50 or less, more preferably 30 or less, and still more preferably 20 nucleotides or less. In general, such a nucleotide sequence includes at least one variance site, preferably a variance site which is informative with respect to the expected response of a patient to a treatment as described for above aspects.

As indicated above, many therapeutic compounds or combinations of compounds or pharmaceutical compositions show variable efficacy and/or safety in various patients in whom the compound or compounds is administered. Thus, it is beneficial to identify variances in relevant genes, e.g., genes related to the action or toxicity of the compound or compounds. Thus, in a further aspect, the invention provides a method for determining whether a compound has a differential effect due to the presence or absence of at least one variance in a gene or a variant form of a gene, where the gene is a gene identified for aspects above.

The method involves identifying a first patient or set of patients suffering from a disease or condition whose response to a treatment differs from the response (to the same treatment) of a second patient or set of patients suffering from the same disease or condition, and then determining whether the occurrence or frequency of occurrence of at least one variance in at least one gene differs between the first patient or set of patients and the second patient or set of patients. A correlation between the presence or absence of the variance or variances and the response of the patient or patients to the treatment indicates that the variance provides information about variable patient response. In general, the method will involve identifying at least one variance in at least one gene. An alternative approach is to identify a first patient or set of patients suffering from a disease or condition and having a particular genotype, haplotype or combination of genotypes or haplotypes, and a second patient or set of patients suffering from the same disease or condition that have a genotype or haplotype or sets of genotypes or haplotypes that differ in a specific way from those of the first set of patients. Subsequently the extent and magnitude of clinical response can be compared between the first patient or set of patients and the second patient or set of patients. A correlation between the presence or absence of a variance or variances or haplotypes and the response of the patient or patients to the treatment indicates that the variance provides information about variable patient response and is useful for the present invention.

The method can utilize a variety of different informative comparisons to identify correlations. For example a plurality of pairwise comparisons of treatment response and the presence or absence of at least one variance can be performed for a plurality of patients. Likewise, the method can involve comparing the response of at least one patient homozygous for at least one variance with at least one patient homozygous for the alternative form of that variance or variances. The method can also involve comparing the response of at least one patient heterozygous for at least one variance with the response of at least one patient homozygous for the at least one variance. Preferably the heterozygous patient response is compared to both alternative homozygous forms, or the response of heterozygous patients is grouped with the response of one class of homozygous patients and said group is compared to the response of the alternative homozygous group.

Such methods can utilize either retrospective or prospective information concerning treatment response variability. Thus, in a preferred embodiment, it is previously known that patient response to the method of treatment is variable.

Also in preferred embodiments, the disease or condition is as for other aspects of this invention; for example, the treatment involves administration of a compound or pharmaceutical composition.

In preferred embodiments, the method involves a clinical trial, e.g., as described herein. Such a trial can be arranged, for example, in any of the ways described herein, e.g., in the Detailed Description.

The present invention also provides methods of treatment of a disease or condition, preferably a disease or condition related to a neurological or psychiatric disease or other neurological or psychiatric clinical symptomatology. Such methods combine identification of the presence or absence of particular variances, preferably in a gene or genes described in U.S. patent application Ser. No. 09/689,506, with the administration of a compound; identification of the presence of particular variances with selection of a method of treatment 30 and administration of the treatment; and identification of the presence or absence of particular variances with elimination of a method of treatment based on the variance information indicating that the treatment is likely to be ineffective or contra-indicated, and thus selecting and administering an alternative treatment effective against the disease or condition. Thus, preferred embodiments of these methods incorporate preferred embodiments of such methods as described for such sub-aspects.

As used herein, a “gene” is a sequence of DNA present in a cell that directs the expression of a “biologically active” molecule or “gene product”, most commonly by transcription to produce RNA and translation to produce protein. The “gene product” is most commonly a RNA molecule or protein or a RNA or protein that is subsequently modified by reacting with, or combining with, other constituents of the cell. Such modifications may include, without limitation, modification of proteins to form glycoproteins, lipoproteins, and phosphoproteins, or other modifications known in the art. RNA may be modified without limitation by polyadenylation, splicing, capping or export from the nucleus or by covalent or noncovalent interactions with proteins. The term “gene product” refers to any product directly resulting from transcription of a gene. In particular this includes partial, precursor, and mature transcription products (i.e., pre-mRNA and mRNA), and translation products with or without further processing including, without limitation, lipidation, phosphorylation, glycosylation, or combinations of such processing

The term “gene involved in the origin or pathogenesis of a disease or condition” refers to a gene that harbors mutations or polymorphisms that contribute to the cause of disease, or variances that affect the progression of the disease or expression of specific characteristics of the disease. The term also applies to genes involved in the synthesis, accumulation, or elimination of products that are involved in the origin or pathogenesis of a disease or condition including, without limitation, proteins, lipids, carbohydrates, hormones, or small molecules.

The term “gene involved in the action of a drug” refers to any gene whose gene product affects the efficacy or safety of the drug or affects the disease process being treated by the drug, and includes, without limitation, genes that encode gene products that are targets for drug action, gene products that are involved in the metabolism, activation or degradation of the drug, gene products that are involved in the bioavailability or elimination of the drug to the target, gene products that affect biological pathways that, in turn, affect the action of the drug such as the synthesis or degradation of competitive substrates or allosteric effectors or rate-limiting reaction, or, alternatively, gene products that affect the pathophysiology of the disease process via pathways related or unrelated to those altered by the presence of the drug compound. (Particular variances in the latter category of genes may be associated with patient groups in whom disease etiology is more or less susceptible to amelioration by the drug. For example, there are several pathophysiological mechanisms in hypertension, and depending on the dominant mechanism in a given patient, that patient may be more or less likely than the average hypertensive patient to respond to a drug that primarily targets one pathophysiological mechanism. The relative importance of different pathophysiological mechanisms in individual patients is likely to be affected by variances in genes associated with the disease pathophysiology.) The “action” of a drug refers to its effect on biological products within the body. The action of a drug also refers to its effects on the signs or symptoms of a disease or condition, or effects of the drug that are unrelated to the disease or condition leading to unanticipated effects on other processes. Such unanticipated processes often lead to adverse events or toxic effects. The terms “adverse event” or “toxic″ event” are known in the art and include, without limitation, those listed in the FDA reference system for adverse events.

In accordance with the aspects above and the Detailed Description below, there is also described for this invention an approach for developing drugs that are explicitly indicated for, and/or for which approved use is restricted to individuals in the population with specific variances or combinations of variances, as determined by diagnostic tests for variances or variant forms of certain genes involved in the disease or condition or involved in the action or metabolism or transport of the drug. Such drugs may provide more effective treatment for a disease or condition in a population identified or characterized with the use of a diagnostic test for a specific variance or variant form of the gene if the gene is involved in the action of the drug or in determining a characteristic of the disease or condition. Such drugs may be developed using the diagnostic tests for specific variances or variant forms of a gene to determine the inclusion of patients in a clinical trial.

Thus, the invention also provides a method for producing a pharmaceutical composition by identifying a compound which has differential activity or effectiveness against a disease or condition in patients having at least one variance in a gene, preferably in a gene described in U.S. patent application Ser. No. xxxxx, compounding the pharmaceutical composition by combining the compound with a pharmaceutically acceptable carrier, excipient, or diluent such that the composition is preferentially effective in patients who have at least one copy of the variance or variances. In some cases, the patient has two copies of the variance or variances. In preferred embodiments, the disease or condition, gene or genes, variances, methods of administration, or method of determining the presence or absence of variances is as described for other aspects of this invention. In preferred embodiments, the active component of the pharmaceutical composition is a compound listed in the compound tables of U.S. patent application Ser. No., or a compound chemically related to one of the listed compounds.

Similarly, the invention provides a method for producing a pharmaceutical agent by identifying a compound which has differential activity against a disease or condition in patients having at least one copy of a form of a gene, preferably a gene described in U.S. patent application serial no., having at least one variance and synthesizing the compound in an amount sufficient to provide a pharmaceutical effect in a patient suffering from the disease or condition. The compound can be identified by conventional screening methods and its activity confirmed. For example, compound libraries can be screened to identify compounds which differentially bind to products of variant forms of a particular gene product, or which differentially affect expression of variant forms of the particular gene, or which differentially affect the activity of a product expressed from such gene. Alternatively, the design of a compound can exploit knowledge of the variances provided herein to avoid significant allele specific effects, in order to reduce the likelihood of significant pharmacogenetic effects during the clinical development process. Preferred embodiments are as for the preceding aspect.

In another aspect, the invention provides a method of treating a disease or condition in a patient by selecting a patient whose cells have an allele of an identified gene, preferably a gene selected from the genes listed in Table 1. The allele contains at least one variance correlated with more effective response to a treatment of said disease or condition. The method also includes altering the level of activity in cells of the patient of a product of the allele, where the altering provides a therapeutic effect.

Preferably the allele contains a variance as shown in U.S. patent application Ser. No. 09/689,506, or in Table 1 or 3 of Stanton et al., U.S. application Ser. No. 09/300,747. Also preferably, the altering involves administering to the patient a compound preferentially active on at least one but less than all alleles of the gene.

Preferred embodiments include those as described above for other aspects of treating a disease or condition.

As recognized by those skilled in the art, all the methods of treating described herein include administration of the treatment to a patient.

In a further aspect, the invention provides a method for determining a method of treatment effective to treat a disease or condition by altering the level of activity of a product of an allele of a gene selected from the genes listed in U.S. patent application Ser. No. 09/689,506, and determining whether that alteration provides a differential effect to(with respect to reducing or alleviating a disease or condition, or with respect to variation in toxicity or tolerance to a treatment) in patients with at least one copy of at least one allele of the gene as compared to patients with at least one copy of one alternative allele. The presence of such a differential effect indicates that altering the level of activity of the gene provides at least part of an effective treatment for the disease or condition.

Preferably the method for determining a method of treatment is carried out in a clinical trial, e.g., as described above and/or in the Detailed Description below.

In still another aspect, the invention provides a method for performing a clinical trial or study, which includes selecting or stratifying subjects in the trial or study using a variance or variances or haplotypes from one or more genes specified in U.S. patent application Ser. No. 09/689,506. Preferably the differential efficacy, tolerance, or safety of a treatment in a subset of patients who have a particular variance, variances, or haplotype in a gene or genes from U.S. patent application Ser. No. 09/689,506 is determined by conducting a clinical trial and using a statistical test to assess whether a relationship exists between efficacy, tolerance, or safety and the presence or absence of any of the variances or haplotype in one or more of the genes. Results of the clinical trial or study are indicative of whether a higher or lower efficacy, tolerance, or safety of the treatment in a subset of patients is associated with any of the variance or variances or haplotype in one or more of the genes. In preferred embodiments, the clinical trial or study is a Phase I, II, III, or IV trial or study. Preferred embodiments include the stratifications and/or statistical analyses as described below in the Detailed Description.

In preferred embodiments, normal subjects or patients are prospectively stratified by genotype in different genotype-defined groups, including the use of genotype as a enrollment criterion, using a variance, variances or haplotypes from U.S. patent application Ser. No. 09/689,506, and subsequently a biological or clinical response variable is compared between the different genotype-defined groups. In preferred embodiments, normal subjects or patients in a clinical trial or study are stratified by a biological or clinical response variable in different biologically or clinically-defined groups, and subsequently the frequency of a variance, variances or haplotypes described in U.S. patent application Ser. No. 09/689,506 is measured in the different biologically or clinically defined groups.

In preferred embodiments, e.g., of the above two analyses (and in other aspects of this invention involving patient or normal subject stratification), the normal subjects or patients in a clinical trial or study are stratified by at least one demographic characteristic selected from the groups consisting of sex, age, racial origin, ethnic origin, or geographic origin.

Generally the method will involve assigning patients or subjects to a group to receive the method of treatment or to a control group.

The present invention provides a method for treating a patient at risk for a disease or condition (for example to prevent or delay the onset of frank disease) or a patient already diagnosed with a disease or a disease associated with pathology. The methods include identifying such a patient and determining the patient's genotype or haplotype for an identified gene or genes. The patient identification can, for example, be based on clinical evaluation using conventional clinical metrics and/or on evaluation of a genetic variance or variances in one or more genes, preferably a gene or genes described in U.S. patent application Ser. No. 09/689,506. The invention provides a method for using the patient's genotype status to determine a treatment protocol that includes a prediction of the efficacy and/or safety of a therapy.

In another related aspect, the invention provides a method for identifying a patient for participation in a clinical trial of a therapy for the treatment of a neurological or psychiatric disease or an associated neuropathological or psychiatric condition. The method involves determining the genotype or haplotype of a patient awith (or at risk for) a disease. Preferably the genotype is for a variance in a gene as described in U.S. patent application Ser. No. 09/689,506. Patients with eligible genotypes are then assigned to a treatment or placebo group, preferably by a blinded randomization procedure. In preferred embodiments, the selected patients have, no copies, or at least one copy or two copies of a wild type allele of an identified gene or genes identified in U.S. patent application Ser. No. 09/689,506. Alternatively, patients selected for the clinical trial may have zero, one or two copies of an allele belonging to a set of alleles, where the set of alleles comprise a group of related alleles. One procedure for rigorously defining a set of alleles is by applying phylogenetic methods to the analysis of haplotypes. (See, for example: Templeton A. R., Crandall K. A. and C. F. Sing, A cladistic analysis of phenotypic associations with haplotypes inferred from restriction endonuclease mapping and DNA sequence data. III. Cladogram estimation. Genetics 1992 Oct. 132(2):619-33.) Regardless of the specific tools used to group alleles, the trial would then test the hypothesis that a statistically significant difference in response to a treatment can be demonstrated between two groups of patients each defined by the presence of zero, one or two alleles (or allele groups) at a gene or genes. Said response may be a desired or an undesired response. In a preferred embodiment, the treatment protocol involves a comparison of placebo vs. treatment response rates in two or more genotype-defined groups. For example, a group with no copies of an allele may be compared to a group with two copies, or a group with no copies may be compared to a group consisting of those with one or two copies. In this manner different genetic models (dominant, co-dominant, recessive) for the transmission of a treatment response trait can be tested. Alternatively, statistical methods that do not posit a specific genetic model, such as contingency tables, can be used to measure the effects of an allele on treatment response.

In another preferred embodiment, patients in a clinical trial can be grouped (at the end of the trial) according to treatment response, and statistical methods can be used to compare allele (or genotype or haplotype) frequencies in two groups. For example, responders can be compared to nonresponders, or patients suffering adverse events can be compared to those not experiencing such effects. Alternatively response data can be treated as a continuous variable and the ability of genotype to predict response can be measured. In a preferred embodiment patients who exhibit extreme phenotypes are compared with all other patients or with a group of patients who exhibit a divergent extreme phenotype. For example if there is a continuous or semi-continuous measure of treatment response (for example the Alzheimer's Disease Assessment Scale, the Mini-Mental State Examination or the Hamilton Depression Rating Scale) then the 10% of patients with the most favorable responses could be compared to the 10% with the least favorable, or the patients one standard deviation above the mean score could be compared to the remainder, or to those one standard deviation below the mean score. One useful way to select the threshold for defining a response is to examine the distribution of responses in a placebo group. If the upper end of the range of placebo responses is used as a lower threshold for an ‘outlier response’ then the outlier response group should be almost free of placebo responders. This is a useful threshold because the inclusion of placebo responders in a ‘true’ response group decreases the ability of statistical methods to detect a genetic difference between responders and nonresponders.

In a related aspect, the invention provides a method for developing a disease management protocol that entails diagnosing a patient with a disease or a disease susceptibility, determining the genotype of the patient at a gene or genes correlated with treatment response and then selecting an optimal treatment based on the disease and the genotype (or genotypes or haplotypes). The disease management protocol may be useful in an education program for physicians, other caregivers or pharmacists; may constitute part of a drug label; or may be useful in a marketing campaign.

By “disease management protocol” or “treatment protocol” is meant a means for devising a therapeutic plan for a patient using laboratory, clinical and genetic data, including the patient's diagnosis and genotype. The protocol clarifies therapeutic options and provides information about probable prognoses with different treatments. The treatment protocol may theprovide an estimate of the likelihood that a patient will respond positively or negatively to a therapeutic intervention. The treatment protocol may also provide guidance regarding optimal drug dose and administration, and likely timing of recovery or rehabilitation. A “disease management protocol” or “treatment protocol” may also be formulated for asymptomatic and healthy subjects in order to forecast future disease risks based on laboratory, clinical and genetic variables. In this setting the protocol specifies optimal preventive or prophylactic interventions, including use of compounds, changes in diet or behavior, or other measures. The treatment protocol may include the use of a computer program.

In another aspect, the invention provides a kit containing at least one probe or at least one primer (or other amplification oligonucleotide) or both (e.g., as described above) corresponding to a gene or genes in U.S. patent application Ser. No. 09/689,506 or other gene related to a disease or condition. The kit is preferably adapted and configured to be suitable for identification of the presence or absence of a particular variance or variances, which can include or consist of a nucleic acid sequence corresponding to a portion of a gene. A plurality of variances may comprise a haplotype of haplotypes. The kit may also contain a plurality of either or both of such probes and/or primers, e.g., 2, 3, 4, 5, 6, or more of such probes and/or primers. Preferably the plurality of probes and/or primers are adapted to provide detection of a plurality of different sequence variances in a gene or plurality of genes, e.g., in 2, 3, 4, 5, or more genes or to amplify and/or sequence a nucleic acid sequence including at least one variance site in a gene or genes. Preferably one or more of the variance or variances to be detected are correlated with variability in a treatment response or tolerance, and are preferably indicative of an effective response to a treatment. In preferred embodiments, the kit contains components (e.g., probes and/or primers) adapted or useful for detection of a plurality of variances (which may be in one or more genes) indicative of the effectiveness of at least one treatment, preferably of a plurality of different treatments for a particular disease or condition. It may also be desirable to provide a kit containing components adapted or useful to allow detection of a plurality of variances indicative of the effectiveness of a treatment or treatment against a plurality of diseases. The kit may also optionally contain other components, preferably other components adapted for identifying the presence of a particular variance or variances. Such additional components can, for example, independently include a buffer or buffers, e.g., amplification buffers and hybridization buffers, which may be in liquid or dry form, a DNA polymerase, e.g., a polymerase suitable for carrying out PCR (e.g., a thermostable DNA polymerase), and deoxy nucleotide triphosphates (dNTPs). Preferably a probe includes a detectable label, e.g., a fluorescent label, enzyme label, light scattering label, or other label. Preferably the kit includes a nucleic acid or polypeptide array on a solid phase substrate. The array may, for example, include a plurality of different antibodies, and/or a plurality of different nucleic acid sequences. Sites in the array can allow capture and/or detection of nucleic acid sequences or gene products corresponding to different variances in one or more different genes. Preferably the array is arranged to provide variance detection for a plurality of variances in one or more genes which correlate with the effectiveness of one or more treatments of one or more diseases, which is preferably a variance as described herein.

The kit may also optionally contain instructions for use, which can include a listing of the variances correlating with a particular treatment or treatments for a disease or diseases and/or a statement or listing of the diseases for which a particular variance or variances correlates with a treatment efficacy and/or safety.

Preferably the kit components are selected to allow detection of a variance described herein, and/or detection of a variance indicative of a treatment, e.g., administration of a drug, pointed out herein.

Additional configurations for kits of this invention will be apparent to those skilled in the art.

The invention also includes the use of such a kit to determine the genotype(s) of one or more individuals with respect to one or more variance sites in one or more genes identified herein. Such use can include providing a result or report indicating the presence and/or absence of one or more variant forms or a gene or genes which are indicative of the effectiveness of a treatment or treatments.

In another aspect, the invention provides a method for determining whether there is a genetic component to intersubject variation in a surrogate treatment response. The method involves administering the treatment to a group of related (preferably normal) subjects and a group of unrelated (preferably normal) subjects, measuring a surrogate pharmacodynamic or pharmacokinetic drug response variable in the subjects, performing a statistical test measuring the variation in response in the group of related subjects and, separately in the group of unrelated subjects, comparing the magnitude or pattern of variation in response or both between the groups to determine if the responses of the groups are different, using a predetermined statistical measure of difference. A difference in response between the groups is indicative that there is a genetic component to intersubject variation in the surrogate treatment response.

In preferred embodiments, the size of the related and unrelated groups is set in order to achieve a predetermined degree of statistical power.

In another aspect, the invention provides a method for evaluating the combined contribution of two or more variances to a surrogate drug response phenotype in subjects (preferably normal subjects) by a. genotyping a set of unrelated subjects participating in a clinical trial or study, e.g., a Phase I trial, of a compound. The genotyping is for two or more variances (which can be a haplotype), thereby identifying subjects with specific genotypes, where the two or more specific genotypes define two or more genotype-defined groups. A drug is administered to subjects with two or more of said specific genotypes, and a surrogate pharmacodynamic or pharmacokinetic drug response variable is measured in the subjects. A statistical test or tests is performed to measure response in the groups separately, where the statistical tests provide a measurement of variation in response with each group. The magnitude or pattern of variation in response or both is compared between the groups to determine if the groups are different using a predetermined statistical measure of difference.

In preferred embodiments, the specific genotypes are homozygous genotypes for two variances. In preferred embodiments, the comparison is between groups of subjects differing in three or more variances, e.g., 3, 4, 5, 6, or even more variances.

In another aspect, the invention provides a method for providing contract research services to clients (preferably in the pharmaceutical and biotechnology industries), by enrolling subjects (e.g., normal and/or patient subjects) in a clinical drug trial or study unit (preferably a Phase I drug trial or study unit) for the purpose of genotyping the subjects in order to assess the contribution of genetic variation to variation in drug response, genotyping the subjects to determine the status of one or more variances in the subjects, administering a compound to the subjects and measuring a surrogate drug response variable, comparing responses between two or more genotype-defined groups of subjects to determine whether there is a genetic component to the interperson variability in response to said compound; and reporting the results of the Phase I drug trial to a contracting entity. Clearly, intermediate results, e.g., response data and/or statistical analysis of response or variation in response can also be reported.

In preferred embodiments, at least some of the subjects have disclosed that they are related to each other and the genetic analysis includes comparison of groups of related individuals. To encourage participation of sufficient numbers of related individuals, it can be advantageous to offer or provide compensation to one or more of the related individuals based on the number of subjects related to them who participate in the clinical trial, or on whether at least a minimum number of related subjects participate, e.g., at least 3, 5, 10, 20, or more.

In a related aspect, the invention provides a method for recruiting a clinical trial population for studies of the influence of genetic variation on drug response, by soliciting subjects to participate in the clinical trial, obtaining consent of each of a set of subjects for participation in the clinical trial, obtaining additional related subjects for participation in the clinical trial by compensating one or more of the related subjects for participation of their related subjects at a level based on the number of related subjects participating or based on participation of at least a minimum specified number of related subjects, e.g., at minimum levels as specified in the preceding aspect.

In yet another aspect, the invention provides a method for identifying phenotypes that vary in cell lines as a result of genetic variation, by measuring one or more phenotypes in cell lines from one or more pedigrees, and testing whether the pattern of phenotype data in the cell lines conforms to the rules of Mendelian transmission. Conformation of the phenotype data to the rules of Mendelian transmission is indicative that said phenotype varies in cell lines as a result of genetic variation.

In preferred embodiments, the cell lines are derived from the CEPH pedigrees. In preferred embodiments, the gene or genes responsible for the inter-cell line variation in phenotype are mapped to chromosomal loci by comparison of the pattern of segregation of the phenotype in the cell lines with the pattern of segregation of known mapped variances in the same cell lines.

In preferred embodiments, at least 5 cell lines from related individuals are tested, preferably at least 50, 100, 200, 300, 400, 500 or even more cell lines are tested. In preferred embodiments, the cells are subjected to a treatment before measuring the phenotype. The treatment includes one or more of: addition of a compound (e.g., a therapeutic compound) to the cells, change in the nutritional environment of the cells, and change in the physical environment of the cells.

Similar to an aspect described above, in another aspect the invention provides a method for identifying mRNAs that vary in levels as a result of genetically determined regulatory factors, by measuring levels of one or more specific mRNAs in cell lines from one or more pedigrees, and testing whether the mRNA levels of said one or more specific mRNAs in said cell lines conforms to the rules of Mendelian transmission. Conformation of any of the mRNA levels to the rules of Mendelian transmission is indicative that the mRNA level varies in cell lines as a result of genetic variation. Preferably the cell lines are derived from the CEPH pedigrees.

In preferred embodiments, the gene or genes responsible for the intersubject variation in levels of specific mRNAs are mapped to chromosomal loci by comparison of the pattern of segregation of the mRNA levels in the cell lines with the pattern of segregation of variances that are already mapped to the human genome.

In preferred embodiments, at least 100 cell lines from related individuals are tested. In other embodiments, at least 200, 300, 400, 500, or even more cell lines are tested. Also in preferred embodiments, the cells are subjected to a treatment before performing the RNA analysis. The treatment includes one or more of: (a) addition of a compound (e.g., a therapeutic compound) to the cells, (b) change in the nutritional environment of the cells, and (c) change in the physical environment of the cells.

By “pathway” or “gene pathway” is meant the goup of biologically relevant genes involved in a pharmacodynamic or pharmacokinetic mechanism of drug, agent, or candidate therapeutic intervention. These mechanisms may further include any physiologic effect the drug or candidate therapeutic intervention renders. Included in this are “biochemical pathway's” which is used in its usual sense to refer to a series of related biochemical processes (and the corresponding genes and gene products) involved in carrying out a reaction or series of reactions. Generally in a cell, a pathway performs a significant process in the cell.

By “pharmacological activity” used herein is meant a biochemical or physiological effect of drugs, compounds, agents, or candidate therapeutic interventions upon administration and the mechanism of action of that effect.

The pharmacological activity is then determined by interactions of drugs, compounds, agents, or candidate therapeutic interventions, or their mechanism of action, on their target proteins or macromolecular components. By “agonist” or “mimetic” or “activators” is meant a drug, agent, or compound that activate physiologic components and mimic the effects of endogenous regulatory compounds. By “antagonists”, “blockers” or “inhibitors” is meant drugs, agents, or compounds that bind to physiologic components and do not mimic endogenous regulatory compounds, or interfere with the action of endogenous regulatory compounds at physiologic components. These inhibitory compounds do not have intrinsic regulatory activity, but prevent the action of agonists. By “partial agonist” or “partial antagonist” is meant an agonist or antagonist, respectively, with limited or partial activity. By “negative agonist” or “inverse antagonists” is meant that a drug, compound, or agent that can interact with a physiologic target protein or macromolecular component and stabilizes the protein or component such that agonist-dependent conformational changes of the component do not occur and agonist mediated mechanism of physiological action is prevented. By “modulators” or “factors” is meant a drug, agent, or compound that interacts with a target protein or macromolecular component and modifies the physiological effect of an agonist.

As used herein the term “chemical class” refers to a group of compounds that share a common chemical scaffold but which differ in respect to the substituent groups linked to the scaffold. Examples of chemical classes of drugs include, for example, phenothiazines, piperidines, benzodiazepines and aminoglycosides. Members of the phenothiazine class include, for example, compounds such as chlorpromazine hydrochoride, mesoridazine besylate, thioridazine hydrochloride, acetophenazine maleate trifluoperazine hydrochloride and others, all of which share a phenothiazine backbone. Members of the piperidine class include, for example, compounds such as meperidine, diphenoxylate and loperamide, as well as phenylpiperidines such as fentanyl, sufentanil and alfentanil, all of which share the piperidine backbone. Chemical classes and their members are recognized by those skilled in the art of medicinal chemistry.

As used herein the term “surrogate marker” refers to a biological or clinical parameter that is measured in place of the biologically definitive or clinically most meaningful parameter. In comparison to definitive markers, surrogate markers are generally either more convenient, less expensive, provide earlier information or provide pharmacological or physiological information not directly obtainable with definitive markers. Examples of surrogate biological parameters: (i) testing erythrocye membrane acetylcholinesterase levels in subjects treated with an acetylcholinesterase inhibitor intended for use in Alzheimer's disease patients (where inhibition of brain acetylcholinesterase would be the definitive biological parameter); (ii) measuring levels of CD4 positive lymphocytes as a surrogate marker for response to a treatment for aquired immune deficiency syndrome (AIDS). Examples of surrogate clinical parameters: (i) performing a psychometric test on normal subjects treated for a short period of time with a candidate Alzheimer's compound in order to determine if there is a measurable effect on cognitive function. The definitive clinical test would entail measurring cognitive function in a clinical trial in Alzheimer's disease patients; (ii) measuring blood pressure as a surrogate marker for myocardial infarction. The measurement of a surrogate marker or parameter may be an endpoint in a clinical study or clinical trial, hence “surrogate endpoint”.

As used herein the term “related” when used with respect to human subjects indicates that the subjects are known to share a common line of descent; that is, the subjects have a known ancestor in common. Examples of preferred related subjects include sibs (brothers and sisters), parents, grandparents, children, grandchildren, aunts, uncles, cousins, second cousins and third cousins. Subjects less closely related than third cousins are not sufficiently related to be useful as “related” subjects for the methods of this invention, even if they share a known ancestor, unless some related individuals that lie between the distantly related subjects are also included. Thus, for a group of related individuals, each subject shares a known ancestor within three generations or less with at least one other subject in the group, and preferably with all other subjects in the group or has at least that degree of consanguinity due to multiple known common ancestors. More preferably, subjects share a common ancestor within two generations or less, or otherwise have equivalent level of consanguinity. Conversely, as used herein the term “unrelated”, when used in respect to human subjects, refers to subjects who do not share a known ancestor within 3 generations or less, or otherwise have known relatedness at that degree.

As used herein the term “pedigree” refers to a group of related individuals, usually comprising at least two generations, such as parents and their children, but often comprising three generations (that is, including grandparents or grandchildren as well). The relation between all the subjects in the pedigree is known and can be represented in a genealogical chart.

As used herein the term “hybridization”, when used with respect to DNA fragments or polynucleotides encompasses methods including both natural polynucleotides, non-natural polynucleotides or a combination of both. Natural polynucleotides are those that are polymers of the four natural deoxynucleotides (deoxyadenosine triphosphate [dA], deoxycytosine triphosphate [dC], deoxyguanine triphosphate [dG] or deoxythymidine triphosphate [dT], usually designated simply thymidine triphosphate [T]) or polymers of the four natural ribonucleotides (adenosine triphosphate [A], cytosine triphosphate [C], guanine triphosphate [G] or uridine triphosphate [U]). Non-natural polynucleotides are made up in part or entirely of nucleotides that are not natural nucleotides; that is, they have one or more modifications. Also included among non-natural polynucleotides are molecules related to nucleic acids, such as peptide nucleic acid [PNA]). Non-natural polynucleotides may be polymers of non-natural nucleotides, polymers of natural and non-natural nucleotides (in which there is at least one non-natural nucleotide), or otherwise modified polynucleotides. Non-natural polynucleotides may be useful because their hybridization properties differ from those of natural polynucleotides. As used herein the term “complementary”, when used in respect to DNA fragments, refers to the base pairing rules established by Watson and Crick: A pairs with T or U; G pairs with C, Complementary DNA fragments have sequences that, when aligned in antiparallel orientation, conform to the Watson-Crick base pairing rules at all positions or at all positions except one. As used herein, complementary DNA fragments may be natural polynucleotides, non-natural polynucleotides, or a mixture of natural and non-natural polynucleotides.

As used herein “amplify” when used with respect to DNA refers to a family of methods for increasing the number of copies of a starting DNA fragment. Amplification of DNA is often performed to simplify subsequent determination of DNA sequence, including genotyping or haplotyping. Amplification methods include the polymerase chain reaction (PCR), the ligase chain reaction (LCR) and methods using Q beta replicase, as well as transcription-based amplification systems such as the isothermal amplification procedure known as self-sustained sequence replication (3SR, developed by T. R. Gingeras and colleagues), strand displacement amplification (SDA, developed by G. T. Walker and colleagues) and the rolling circle amplification method (developed by P. Lizardi and D. Ward).

As used herein “contract research services for a client” refers to a business arrangement wherein a client entity pays for services consisting in part or in whole of work performed using the methods described herein. The client entity may include a commercial or non-profit organization whose primary business is in the pharmaceutical, biotechnology, diagnostics, medical device or contract research organization (CRO) sector, or any combination of those sectors. Services provided to such a client may include any of the methods described herein, particularly including clinical trial services, and especially the services described in the Detailed Description relating to a Pharmacogenetic Phase I Unit. Such services are intended to allow the earliest possible assessment of the contribution of a variance or variances or haplotypes, from one or more genes, to variation in a surrogate marker in humans. The surrogate marker is generally selected to provide information on a biological or clinical response, as defined above.

As used herein, “comparing the magnitude or pattern of variation in response” between two or more groups refers to the use of a statistical procedure or procedures to measure the difference between two different distributions. For example, consider two genotype-defined groups, AA and aa, each homozygous for a different variance or haplotype in a gene believed likely to affect response to a drug. The subjects in each group are subjected to treatment with the drug and a treatment response is measured in each subject (for example a surrogate treatment response). One can then construct two distributions: the distribution of responses in the AA group and the distribution of responses in the aa group. These distributions may be compared in many ways, and the significance of any difference qualified as to its significance (often expressed as a p value), using methods known to those skilled in the art. For example, one can compare the means, medians or modes of the two distributions, or one can compare the variance or standard deviations of the two distributions. Or, if the form of the distributions is not known, one can use nonparametric statistical tests to test whether the distributions are different, and whether the difference is significant at a specified level (for example, the p<0.05 level, meaning that, by chance, the distributions would differ to the degree measured less than one in 20 similar experiments). The types of comparisons described are similar to the analysis of heritability in quantitative genetics, and would draw on standard methods from quantitative genetics to measure heritability by comparing data from related subjects.

Another type of comparison that can be usefully made is between related and unrelated groups of subjects. That is, the comparison of two or more distributions is of particular interest when one distribution is drawn from a population of related subjects and the other distribution is drawn from a group of unrelated subjects, both subjected to the same treatment. (The related subjects may consist of small groups of related subjects, each compared only to their relatives.) A comparison of the distribution of a drug response variable (e.g. a surrogate marker) between two such groups may provide information on whether the drug response variable is under genetic control. For example, a narrow distribution in the group(s) of related subjects (compared to the unrelated subjects) would tend to indicate that the measured variable is under genetic control (i.e. the related subjects, on account of their genetic homogeneity, are more similar than the unrelated individuals). The degree to which the distribution was narrower in the related individuals (compared to the unrelated individuals) would be proportionate to the degree of genetic control. The narrowness of the distribution could be quantified by, for example, computing variance or standard deviation. In other cases the shape of the distribution may not be known and nonparametric tests may be preferable. Nonparametric tests include methods for comparing medians such as the sign test, the slippage test, or the rank correlation coefficient (the nonparametric equivalent of the ordinary correlation coefficient). Pearson's Chi square test for comparing an observed set of frequencies with an expected set of frequencies can also be useful.

The present invention provides a number of advantages. For example, the methods described herein allow for use of a determination of a patient's genotype for the timely administration of the most suitable therapy for that particular patient. The methods of this invention provide a basis for successfully developing and obtaining regulatory approval for a compound even though efficacy or safety of the compound in an unstratified population is not adequate to justify approval. From the point of view of a pharmaceutical or biotechnology company, the information obtained in pharmacogenetic studies of the type described herein could be the basis of a marketing campaign for a drug. For example, a marketing campaign that emphasized the superior efficacy or safety of a compound in a genotype or haplotype restricted patient population, compared to a similar or competing compound used in an undifferentiated population of all patients with the disease. In this respect a marketing campaign could promote the use of a compound in a genetically defined subpopulation, even though the compound was not intrinsically superior to competing compounds when used in the undifferentiated population with the target disease. In fact even a compound with an inferior profile of action in the undifferentiated disease population could become superior when coupled with the appropriate pharmacogenetic test.

By “comprising” is meant including, but not limited to, whatever follows the word “comprising”. Thus, use of the term “comprising” indicates that the listed elements are required or mandatory, but that other elements are optional and may or may not be present. By “consisting of ” is meant including, and limited to, whatever follows the phrase “consisting of ”. Thus, the phrase “consisting of ” indicates that the listed elements are required or mandatory, and that no other elements may be present. By “consisting essentially of ” is meant including any elements listed after the phrase, and limited to other elements that do not interfere with or contribute to the activity or action specified in the disclosure for the listed elements. Thus, the phrase “consisting essentially of ” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DETAILED DESCRIPTION OF THE PREFERRED EMBODIMENTS

### I. Identification of Interpatient Variation in Response; Identification of Genes and

### Variances Relevant to Drug Action; Development of Diagnostic Tests; and Use of Variance Status to Determine Treatment

Development of therapeutics in man follows a course from compound discovery and analysis in a laboratory (preclinical development) to testing the candidate therapeutic intervention in human subjects (clinical development). The preclinical development of candidate therapeutic interventions for use in the treatment of human diseases, disorders, or conditions begins at the discovery stage whereby a candidate therapy is tested in vitro to achieve a desired biochemical alteration of a biochemical or physiological event. If successful, the candidate is generally tested in animals to determine toxicity, adsorption, distribution, metabolism and excretion in a living species. Occasionally, there are available animal models that mimic human diseases, disorders, and conditions in which testing the candidate therapeutic intervention can provide supportive data to warrant proceeding to test the compound in humans. It is widely recognized that preclinical data is imperfect in predicting response to a compound in man. Both safety and efficacy have to ultimately be demonstrated in humans. Therefore, given economic constraints, and considering the complexities of human clinical trials, any technical advance that increases the likelihood of successfully developing and registering a compound, or getting new indications for a compound, or marketing a compound successfully against competing compounds or treatment regimens, will find immediate use. Indeed, there has been much written about the potential of pharmacogenetics to change the practice of medicine. In this application we provide descriptions of the methods one skilled in the art would use to advance compounds through clinical trials using genetic stratification as a tool to circumvent some of the difficulties typically encountered in clinical development, such as poor efficacy or toxicity. We also provide specific genes, variation in which may account for interpatient variation in treatment response, and further we provide specific exemplary variances in those genes that may account for variation in treatment response.

The study of sequence variation in genes that mediate and modulate the action of drugs may provide advances at virtually all stages of drug development. For example, identification of amino acid variances in a drug target during preclinical development would allow development of non-allele selective agents. During early clinical development, knowledge of variation in a gene related to drug action could be used to design a clinical trial parametersin which the variances are taken account of by, for example, including secondary endpoints that incorporate an analysis of response rates in genetic subgroups. In later stages of clinical development the goal might be to first establish retrospectively whether a particular problem, such as liver toxicity, can be understood in terms of genetic subgroups, and thereby controlled using a genetic test to screen patients. Thus genetic analysis of drug response can aid successful development of therapeutic products at any stage of clinical development. Even after a compound has achieved regulatory approval its commercialization can be aided by the methods of this invention, for example by allowing identification of genetically defined responder subgroups in new indications (for which approval in the entire disease population could not be achieved) or by providing the basis for a marketing campaign that highlights the superior efficacy and/or safety of a compound coupled with a genetic test to identify preferential responders. Thus the methods of this invention will provide medical, economic and marketing advantages for products, and over the longer term increase therapeutic alternatives for patients.

### Advantages of Pharmacogenomic Clinical Development of Novel Candidate Therapeutic Interventions for Disease

The evidence that a variance in a gene involved in a pathway that affects drug response, indicates and supports the theory that there is a likelihood that other genes have similar qualities to various degrees. As drug research and development proceeds to identify more lead candidate therapeutic interventions for neurologic and psychiatric disease, there is possible utility in stratifying patients based upon their genotype for these yet to be correlated variances. Further, as described in the Detailed Description, methods for the identification of candidate genes and gene pathways, stratification, clinical trial design, and implementation of genotyping for appropriate medical management of a given disease is easily translated for patients with neurologic and psychiatric disease. As described below there are likely gene pathways as are those that are outlined in U.S. patent application serial no. xxxxx.

The advantages of a clinical research and drug development program that include the use of polymorphic genotyping for the stratification of patients for the appropriate selection of candidate therapeutic intervention includes 1) identification of patients that may respond earlier to therapy, 2) identification of the primary gene and relevant polymorphic variance that directly affects efficacy, safety, or both, 3) identification of pathophysiologic relevant variance or variances and potential therapies affecting those allelic genotypes or haplotypes, and 4) identification of allelic variances or haplotypes in genes that indirectly affects efficacy, safety or both.

Based upon these advantages, designing and performing a clinical trial, either prospective or retrospective, which includes a genotype stratification arm will incorporate analysis of clinical outcomes and potential genetic variation associated with those outcomes, and hypothesis testing of the statistically relevant correlation of the genotypic stratification and therapeutic benefits. If statistical relevance is detectable, these studies will be incorporated into regulatory filings. Ultimately, these clinical trial data will be considered during the approval for marketing process, as well as, incorporated into accepted medical management of anxiety.

By identifying subsets of patients diagnosed with anxiety that respond earlier to agents, optimal candidate therapeutic interventions may reduce the lag time prior to relief of psychiatric symptoms. Appropriate genotyping and correlation to dosing regimen would be beneficial to the patient, caregivers, medical personnel, and the patient's loved ones.

As an example of identification of the primary gene and relevant polymorphic variance that directly affects efficacy, safety, or both one could select a gene pathway as described in the Detailed Description, and determine the effect of genetic polymorphism and therapy efficacy, safety, or both within that given pathway. By embarking on the previously described gene pathway approach, it is technically feasible to determine the relevant genes within such a targeted drug development program for neurologic or psychiatric disease.

Identification of pathophysiologic relevant variance or variances and potential therapies affecting those allelic genotypes or haplotypes will speed the drug development. There is a need for therapies that are targeted to the disease and symptom management with limited or no undesirable side effects. Identification of a specific variance or variances within genes involved in the pathophysiologic manifestation of anxiety and specific genetic polymorphisms of these critical genes can assist the development of novel anxiolytic agents and the identification of those patients that may best benefit from therapy of these candidate therapeutic alternatives.

By identifying allelic variances or haplotypes in genes that indirectly affects efficacy, safety or both one could target specific secondary drug or agent therapeutic actions that affect the overall therapeutic action of conventional, atypical, or novel action.

In U.S. patent application Ser. No. 09/689,506, there is a listing of candidate genes and specific single nucleotide polymorphisms that may be critical for the identification and stratification of an anxiety patient population based upon genotype. One skilled in the art would be able to identify these pathway specific genes or other genes that may be involved in the manifestation of neurologic or psychiatric disease or are likely candidate targets for therapeutic approaches described in this invention.

A sample of therapies approved or in development for preventing or treating the progression of symptoms of neurologic and psychiatric disease currently known in the art is shown in U.S. patent application Ser. No. 09/689,506. In these tables, the candidate therapeutics were sorted and listed by mechanism of action. Further, the product name, the pharmacologic mechanism of action, chemical name (if specified), and the indication is listed as well.

Pharmacogenomics studies for these drugs, as well as other agents, drugs, compounds or candidate therapeutic interventions, could be performed by identifying genes that are involved in the function of a drug including, but not limited to is absorption, distribution metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions, which are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

For each of the described neurologic or psychiatric disease indications one skilled in the art can identify novel candidate therapeutic interventions that may be used to treat the disease or symptoms and/or proceed with a regimen of palliative care. For compounds that have yet to achieve approval, or are still in development one skilled in the art can determine those candidate therapeutic interventions that may be of therapuetic benefit.

### Exemplary Compounds in Development for Disease Management

There are many sources for obtaining information on drugs approved for human therapeutic use an for those compounds under clinical or preclinical investigation, as well as for compounds which have been identified as having a particular pharmacological activity. For products, which have been approved, the PDR contains a listing of the package inserts for all of the products available for human therapeutic intervention. The Merck Index can be used as an additional text to supplement information gathered on the candidate therapeutic interventions. For products that are under clinical or preclinical development, there are databases cataloging information on those candidate therapeutic interventions. Generally that information includes aspects of the drug development process, such as phase of development, identified therapeutic indications, name of manufacturer, mechanistic and pharmacological activities of the product. These databases are available for a fee, and include: PharmaProjects (http://pjbpubs.co.uklpharmamain2/html) and R&D Focus (http://www.ims.global.com/products/lifecycle/r_and_d.htm). One skilled in the art can readily utilize these sources to determine appropriate candidate therapeutic intervention for the identified disease, disorder or condition.

Since there are a large number of candidate therapeutic interventions that are either approved for human therapeutic use or under clinical or preclinical investigation, one skilled in the art could search through publicly available or fee-for-access databases for interventions that may be of therapeutic benefit for a particular disease, disorder, or condition, and determine whether variances in particular genes correlate with interpatient variation in response to one or more of those therapeutic interventions. An example of the results of such searching is provided in U.S. patent application Ser. No. xxxxx. In these tables, the disease, disorder or condition is listed. In order to generate a table or other compendium of information as listed in the table, one skilled in the art can search, for example, in databases for products having the indication “schizophrenia”. Alternatively, one can search for alternative indications or co-morbidities, e.g., pyschoses, neuroleptic, neurological to arrive at a more complete list of the available products. In the table, the candidate therapeutics were sorted and listed by pharmacologic mechanism of action (action). Further, the product name, chemical name (if specified), as well as the indication considered for clinical development. If the candidate therapeutic interventions are approved for therapeutic use, then one skilled in the art can obtain dosing, adverse events, pharmacologic parameters (both pharmacokinetic and pharmacodynamic), and clinical data or information by referring to the PDR. If the candidate therapeutic intervention are in clinical or preclinical stages of drug development, then one skilled in the art would gather data on dosing, adverse events, pharmacologic parameters (both pharmacokinetic and pharmacodynamic), and clinical data or information for the drug or product sponsor. In both cases, selection of a candidate therapeutic intervention for retrospective or prospective pharmacogenetic clinical studies would use an analysis of the likelihood that there is a phenomenological or statistical support for the review of the data to ascertain whether the candidate therapeutic intervention (approved or in development) efficacy or safety profiles can be grouped based upon the individual's genotype or phenotype. In this way, a gene or genes selected, e.g., from a pathway involving the cellular or more broadly the pharmacological mechanism of actions, can be identified and genotyping can be performed in order to determine the allelic variance, variances, for haplotype. Further, one could group the individuals by such genetic variances and further by the therapeutic outcome determinants.

Pharmacogenomics studies for these drugs, as well as other agents, drugs, compounds or candidate therapeutic interventions, can be performed by identifying genes that are involved in the the function of a drug including, but not limited to is absorption, distribution metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions, which are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

Further, there may be genes within pathways that are either involved in metabolism of neurotransmitters or are involved in metabolism of various drugs or compounds. In U.S. patent application Ser. No. 09/689,506, there are listings of candidate genes and specific single nucleotide polymorphisms that may be critical for the identification and stratification of a patient population diagnosed with neurologic or psychiatric disease based upon genotype. Current pathways that may have involvement in the therapeutic benefit of neurologic or psytchiatric disease are listed as gene pathways and are listed in U.S. patent application Ser. No. 09/689,506. One skilled in the art would be able to identify these pathway specific gene or genes that may be involved in the manifestation of the described disease, are likely candidate targets for novel therapeutic approaches, or are involved in mediating patient population differences in drug response to therapies for neurological or psychiatric disease described in the present invention.

As certain aspects of the present invention typically involve the following process, which need not occur separately or in the order stated. Not all of these described processes must be present in a particular method, or need be performed by a single entity or organization or person. Additionally, if certain of the information is available from other sources, that information can be utilized in the present invention. The processes are as follows: a) variability between patients in the response to a particular treatment is observed; b) at least a portion of the variable response is correlated with the presence or absence of at least one variance in at least one gene; c) an analytical or diagnostic test is provided to determine the presence or absence of the at least one variance in individual patients; d) the presence or absence of the variance or variances is used to select a patient for a treatment or to select a treatment for a patient, or the variance information is used in other methods described herein.

### A. Identification of Interpatient Variability in Response to a Treatment

Interpatient variability is the rule, not the exception, in clinical therapeutics. One of the best sources of information on interpatient variability is the nurses and physicians supervising the clinical trial who accumulate a body of first hand observations of physiological responses to the drug in different normal subjects or patients. Evidence of interpatient variation in response can also be measured statistically, and may be best assessed by descriptive statistical measures that examine variation in response (beneficial or adverse) across a large number of subjects, including in different patient subgroups (men vs. women; whites vs. blacks; Northern Europeans vs. Southern Europeans, etc.).

In accord with the other portions of this description, the present invention concerns DNA sequence variances that can affect one or more of:

i. The susceptibility of individuals to a disease;

ii. The course or natural history of a disease;

iii. The response of a patient with a disease to a medical intervention, such as, for example, a drug, a biologic substance, physical energy such as radiation therapy, or a specific dietary regimen. (The terms ‘drug’, ‘compound’ or ‘treatment’ as used herein may refer to any of the foregoing medical interventions.) The ability to predict either beneficial or detrimental responses is medically useful.

Thus variation in any of these three parameters may constitute the basis for initiating a pharmacogenetic study directed to the identification of the genetic sources of interpatient variation. The effect of a DNA sequence variance or variances on disease susceptibility or natural history (i and ii, above) are of particular interest as the variances can be used to define patient subsets which behave differently in response to medical interventions such as those described in (iii). The methods of this invention are also useful in a clinical development program where there is not yet evidence of interpatient variation (perhaps because the compound is just entering clinical trials) but such variation in response can be reliably anticipated. It is more economical to design pharmacogenetic studies from the beginning of a clinical development program than to start at a later stage when the costs of any delay are likely to be high given the resources typically committed to such a program.

In other words, a variance can be useful for customizing medical therapy at least for either of two reasons. First, the variance may be associated with a specific disease subset that behaves differently with respect to one or more therapeutic interventions (i and ii above); second, the variance may affect response to a specific therapeutic intervention (iii above). Consider for exemplary purposes pharmacological therapeutic interventions. In the first case, there may be no effect of a particular gene sequence variance on the observable pharmacological action of a drug, yet the disease subsets defined by the variance or variances differ in their response to the drug because, for example, the drug acts on a pathway that is more relevant to disease pathophysiology in one variance-defined patient subset than in another variance-defined patient subset. The second type of useful gene sequence variance affects the pharmacological action of a drug or other treatment. Effects on pharmacological responses fall generally into two categories; pharmacokinetic and pharmacodynamic effects. These effects have been defined as follows in Goodman and Gilman's Phamacologic Basis of Therapeutics (ninth edition, McGraw Hill, New York, 1986): “Pharmacokinetics” deals with the absorption, distribution, biotransformations and excretion of drugs. The study of the biochemical and physiological effects of drugs and their mechanisms of action is termed “pharmacodynamics.”

Useful gene sequence variances for this invention can be described as variances which partition patients into two or more groups that respond differently to a therapy or that correlate with differences in disease susceptibility or progression, regardless of the reason for the difference, and regardless of whether the reason for the difference is known. The latter is true because it is possible, with genetic methods, to establish reliable associations even in the absence of a pathophysiological hypothesis linking a gene to a phenotype, such as a pharmacological response, disease susceptibility or disease prognosis.

### B. Identification of Specific Genes and Correlation of Variances in Those Genes With Response to Treatment of Diseases or Conditions

It is useful to identify particular genes which do or are likely to mediate the efficacy or safety of a treatment method for a disease or condition, particularly in view of the large number of genes which have been identified and which continue to be identified in humans. As is further discussed in Section C below, this correlation can proceed by different paths. One exemplary method utilizes prior information on the pharmacology or pharmacokinetics or pharmacodynamics of a treatment method, e.g., the action of a drug, which indicates that a particular gene is, or is likely to be, involved in the action of the treatment method, and further suggests that variances in the gene may contribute to variable response to the treatment method. For example if a compound is known to be glucuronidated then a glucuronyltransferase is likely involved. If the compound is a phenol, the likely glucuronyltransferase is UGT1 (either the UGT1*1or UGT1*6 transcripts, both of which catalyze the conjugation of planar phenols with glucuronic acid). Similar inferences can be made for many other biotransformation reactions.

Alternatively, if such information is not known, variances in a gene can be correlated empirically with treatment response. In this method, variances in a gene which exist in a population can be identified. The presence of the different variances or haplotypes in individuals of a study group, which is preferably representative of a population or populations of known geographic, ethnic and/or racial background, is determined. This variance information is then correlated with treatment response of the various individuals as an indication that genetic variability in the gene is at least partially responsible for differential treatment response. It may be useful to independently analyze variances in the different geographic, ethnic and/or racial groups as the presence of different genetic variances in these groups (i.e. different genetic background) may influence the effect of a specific variance. That is, there may be a gene X gene interaction involving one unstudied gene, however the indicated demographic variables may act as a surrogate for the unstudied allele. Statistical measures known to those skilled in the art are preferably used to measure the fraction of interpatient variation attributable to any one variance, or to measure the response rates in different subgroups defined genetically or defined by some combination of genetic, demographic and clinical criteria.

Useful methods for identifying genes relevant to the pharmacological action of a drug or other treatment are known to those skilled in the art, and include review of the scientific literature combined with inteferential or deductive reasoning that one skilled in the art of molecular pharmacology and molecular biology would be capable of; large scale analysis of gene expression in cells treated with the drug compared to control cells; large scale analysis of the protein expression pattern in treated vs. untreated cells, or the use of techniques for identification of interacting proteins or ligand-protein interactions, such as yeast two-hybrid systems.

### C. Development of a Diagnostic Test to Determine Variance Status

In accordance with the description in the Summary above, the present invention generally concerns the identification of variances in genes which are indicative of the effectiveness of a treatment in a patient. The identification of specific variances, in effect, can be used as a diagnostic or prognostic test. Correlation of treatment efficacy and/or toxicity with particular genes and gene families or pathways is provided in Stanton et al., U.S. Provisional Application 60/093,484, filed Jul. 20, 1998, entitled GENE SEQUENCE VARIANCES WITH UTILITY IN DETERMINING THE TREATMENT OF DISEASE (concerns the safety and efficacy of compounds active on folate or pyrimidine metabolism or action) and Stanton, U.S. Provisional Application No. 60/121,047, filed Feb. 22, 1999, entitled GENE SEQUENCE VARIANCES WITH UTILITY IN DETERMINING THE TREATMENT OF DISEASE (concerning Alzheimer's disease and other dementias and cognitive disorders), which are hereby incorporated by reference in their entireties including drawings.

Genes identified in the examples below and in the Tables and Figures can be used in the methods of the present invention. A variety of genes which the inventors realize may account for interpatient variation in response to treatments for neurological and psychiatric diseases, conditions, disorders, and/or the development of same are listed in U.S. patent application Ser. No. 09/689,506. Gene sequence variances in said genes are particularly useful for aspects of the present invention.

Methods for diagnostic tests are well known in the art. Generally in this invention, the diagnostic test involves determining whether an individual has a variance or variant form of a gene that is involved in the disease or condition or the action of the drug or other treatment or effects of such treatment. Such a variance or variant form of the gene is preferably one of several different variances or forms of the gene that have been identified within the population and are known to be present at a certain frequency. In an exemplary method, the diagnostic test involves determining the sequence of at least one variance in at least one gene after amplifying a segment of said gene using a DNA amplification method such as the polymerase chain reaction (PCR). In this method DNA for analysis is obtained by amplifying a segment of DNA or RNA (generally after converting the RNA to EDNA) spanning one or more variances in the gene sequence. Preferably, the amplified segment is <500 bases in length, in an alternative embodiment the amplified segment is <100 bases in length, most preferably <45 bases in length.

In some cases it will be desirable to determine a haplotype instead of a genotype. In such a case the diagnostic test is performed by amplifying a segment of DNA or RNA (cDNA) spanning more than one variance in the gene sequence and preferably maintaining the phase of the variances on each allele. The term “phase” refers to the relationship of variances on a single chromosomal copy of the gene, such as the copy transmitted from the mother (maternal copy or maternal allele) or the father (paternal copy or paternal allele). The haplotyping test may take part in two phases, where first genotyping tests at two or more variant sites reveal which sites are heterozygous in each patient or normal subject. Subsequently the phase of the two or more variant sites can be determined. In performing a haplotyping test preferably the amplified segment is >500 bases in length, more preferably it is >1,000 bases in length, and most preferably it is >2,500 bases in length. One way of preserving phase is to amplify one strand in the PCR reaction. This can be done using one or a pair of oligonucleotide primers that terminate (i.e. have a 3′ end that stops) opposite the variant site, such that one primer is perfectly complementary to one variant form and the other primer is perfectly complementary to the other variant form. Other than the difference in the 3′ most nucleotide the two primers are identical (forming an allelic primer pair). Only one of the allelic primers is used in any PCR reaction, depending on which strand is being amplified. The primer for the opposite strand may also be an allelic primer, or it may prime from a non-polymorphic region of the template. This method exploits the requirement of most polymerases for perfect complementarity at the 3′ terminus of the primer in a primer-template complex. See, for example: Lo Y M, Patel P, Newton C R, Markham A F, Fleming K A and J S Wainscoat. (1991) Direct haplotype determination by double ARMS: specificity, sensitivity and genetic applications. Nucleic Acids Res July 11;19(13):3561-7.

It is apparent that such diagnostic tests are performed after initial identification of variances within the gene, which allows selection of appropriate allele specific primers.

Diagnostic genetic tests useful for practicing this invention belong to two types: genotyping tests and haplotyping tests. A genotyping test simply provides the status of a variance or variances in a subject or patient. For example suppose nucleotide 15of hypothetical gene X on an autosomal chromosome is an adenine (A) or a guanine (G) base. The possible genotypes in any individual are AA, AG or GG at nucleotide 150 of gene X.

In a haplotyping test there is at least one additional variance in gene X, say at nucleotide 810, which varies in the population as cytosine (C) or thymine (T). Thus a particular copy of gene X may have any of the following combinations of nucleotides at positions 150 and 810: 150A-810C, 150A-810T, 150G-810C or 150G-810T. Each of the four possibilities is a unique haplotype. If the two nucleotides interact in either RNA or protein, then knowing the haplotype can be important. The point of a haplotyping test is to determine the haplotypes present in a DNA or cDNA sample (e.g. from a patient). In the example provided there are only four possible haplotypes, but, depending on the number of variances in the gene and their distribution in human populations there may be three, four, five, six or more haplotypes at a given gene. The most useful haplotypes for this invention are those which occur commonly in the population being treated for a disease or condition. Preferably such haplotypes occur in at least 5% of the population, more preferably in at least 10%, still more preferably in at least 20% of the population and most preferably in at least 30% or more of the population. Conversely, when the goal of a pharmacogenetic program is to identify a relatively rare population that has an adverse reaction to a treatment, the most useful haplotypes may be rare haplotypes, which may occur in less than 5%, less than 2%, or even in less than 1% of the population. One skilled in the art will recognize that the frequency of the adverse reaction provides a useful guide to the likely frequency of salient causative haplotypes.

Based on the identification of variances or variant forms of a gene, a diagnostic test utilizing methods known in the art can be used to determine whether a particular form of the gene, containing specific variances or haplotypes, or combinations of variances and haplotypes, is present in at least one copy, one copy, or more than one copy in an individual. Such tests are commonly performed using DNA or RNA collected from blood, cells, tissue scrapings or other cellular materials, and can be performed by a variety of methods including, but not limited to, PCR based methods, hybridization with allele□specific probes, enzymatic mutation detection, chemical cleavage of mismatches, mass spectrometry or DNA sequencing, including minisequencing. Methods for haplotyping are described above. In particular embodiments, hybridization with allele specific probes can be conducted in two formats: (1) allele specific oligonucleotides bound to a solid phase (glass,. silicon, nylon membranes) and the labelled sample in solution, as in many DNA chip applications, or (2) bound sample (often cloned DNA or PCR amplified DNA) and labelled oligonucleotides in solution (either allele specific or short—e.g. 7 mers or 8 mers—so as to allow sequencing by hybridization). Preferred methods for diagnosting testing of variances are described in four patent applications Stanton et al, entitled A METHOD FOR ANALYZING POLYNUCLEOTIDES, Ser. No. 09/394,467; 09/394,457; 09/394,774; and 09/394,387; all filed Sep. 10, 1999. The application of such diagnostic tests is possible after identification of variances that occur in the population. Diagnostic tests may involve a panel of variances from one or more genes, often on a solid support, which enables the simultaneous determination of more than one variance in one or more genes.

### D. Use of Variance Status to Determine Treatment

In U.S. patent application Ser. No. 09/689,506 describes exemplary gene sequence variances in genes and variant forms of these gene that may be determined using diagnostic tests. As indicated in the Summary, such a variance-based diagnostic test can be used to determine whether or not to administer a specific drug or other treatment to a patient for treatment of a disease or condition. Preferably such diagnostic tests are incorporated in texts such as are described in Clinical Diagnosis and Management by Laboratory Methods (19th Ed) by John B. Henry (Editor) W B Saunders Company, 1996; Clinical Laboratory Medicine Clinical Application of Laboratory Data, (6th edition) by R. Ravel, Mosby-Year Book, 1995, or other medical textbooks including, without limitation, textbooks of medicine, laboratory medicine, therapeutics, pharmacy, pharmacology, nutrition, allopathic, homeopathic, and osteopathic medicine; preferably such a test is developed as a ‘home brew’ method by a certified diagnostic laboratory; most preferably such a diagnostic test is approved by regulatory authorities, e.g., by the U.S. Food and Drug Administration, and is incorporated in the label or insert for a therapeutic compound, as well as in the Physicians Desk Reference.

In such cases, the procedure for using the drug is restricted or limited on the basis of a diagnostic test for determining the presence of a variance or variant form of a gene. Alternatively the use of a genetic test may be advised as best medical practice, but not absolutely required, or it may be required in a subset of patients, e.g. those using one or more other drugs, or those with impaired liver or kidney function. The procedure that is dictated or recommended based on genotype may include the route of administration of the drug, the dosage form, dosage, schedule of administration or use with other drugs; any or all of these may require selecting or determination consistent with the results of the diagnostic test or a plurality of such tests. Preferably the use of such diagnostic tests to determine the procedure for administration of a drug is incorporated in a text such as those listed above, or medical textbooks, for example, textbooks of medicine, laboratory medicine, therapeutics, pharmacy, pharmacology, nutrition, allopathic, homeopathic, and osteopathic medicine. As previously stated, preferably such a diagnostic test or tests are required by regulatory authorities and are incorporated in the label or insert as well as the Physicians Desk Reference.

Variances and variant forms of genes useful in conjunction with treatment methods may be associated with the origin or the pathogenesis of a disease or condition. In many useful cases, the variant form of the gene is associated with a specific characteristic of the disease or condition that is the target of a treatment, most preferably response to specific drugs or other treatments. Examples of diseases or conditions ameliorable by the methods of this invention are identified in the Examples and tables below; in general treatment of disease with current methods, particularly drug treatment, always involves some unknown element (involving efficacy or toxicity or both) that can be reduced by appropriate diagnostic methods.

Alternatively, the gene is involved in drug action, and the variant forms of the gene are associated with variability in the action of the drug. For example, in some cases, one variant form of the gene is associated with the action of the drug such that the drug will be effective in an individual who inherits one or two copies of that form of the gene. Alternatively, a variant form of the gene is associated with the action of the drug such that the drug will be toxic or otherwise contra-indicated in an individual who inherits one or two copies of that form of the gene.

In accord with this invention, diagnostic tests for variances and variant forms of genes as described above can be used in clinical trials to demonstrate the safety and efficacy of a drug in a specific population. As a result, in the case of drugs which show variability in patient response correlated with the presence or absence of a variance or variances, it is preferable that such drug is approved for sale or use by regulatory agencies with the recommendation or requirement that a diagnostic test be performed for a specific variance or variant form of a gene which identifies specific populations in which the drug will be safe and/or effective. For example, the drug may be approved for sale or use by regulatory agencies with the specification that a diagnostic test be performed for a specific variance or variant form of a gene which identifies specific populations in which the drug will be toxic. Thus, approved use of the drug, or the procedure for use of the drug, can be limited by a diagnostic test for such variances or variant forms of a gene; or such a diagnostic test may be considered good medical practice, but not absolutely required for use of the drug.

As indicated, diagnostic tests for variances as described in this invention may be used in clinical trials to establish the safety and efficacy of a drug. Methods for such clinical trials are described below and/or are known in the art and are described in standard textbooks. For example, diagnostic tests for a specific variance or variant form of a gene may be incorporated in the clinical trial protocol as inclusion or exclusion criteria for enrollment in the trial, to allocate certain patients to treatment or control groups within the clinical trial or to assign patients to different treatment cohorts. Alternatively, diagnostic tests for specific variances may be performed on all patients within a clinical trial, and statistical analysis performed comparing and contrasting the efficacy or safety of a drug between individuals with different variances or variant forms of the gene or genes. Preferred embodiments involving clinical trials include the genetic stratification strategies, phases, statistical analyses, sizes, and other parameters as described herein.

Similarly, diagnostic tests for variances can be performed on groups of patients known to have efficacious responses to the drug to identify differences in the frequency of variances between responders and non-responders. Likewise, in other cases, diagnostic tests for variance are performed on groups of patients known to have toxic responses to the drug to identify differences in the frequency of the variance between those having adverse events and those not having adverse events. Such outlier analyses may be particularly useful if a limited number of patient samples are available for analysis. It is apparent that such clinical trials can be or are performed after identifying specific variances or variant forms of the gene in the population. In defining outliers it is useful to examine the distribution of responses in the placebo group; outliers should preferably have responses that exceed in magnitude the extreme responses in the placebo group.

The identification and confirmation of genetic variances is described in certain patents and patent applications. The description therein is useful in the identification of variances in the present invention. For example, a strategy for the development of anticancer agents having a high therapeutic index is described in Housman, International Application PCT/US/94 08473 and Housman, INHIBITORS OF ALTERNATIVE ALLELES OF GENES ENCODING PROTEINS VITAL FOR CELL VIABILITY OR CELL GROWTH AS A BASIS FOR CANCER THERAPEUTIC AGENTS, U.S. Pat. No. 5,702,890, issued Dec. 30, 1997, which are hereby incorporated by reference in their entireties. Also, a number of gene targets and associated variances are identified in Housman et al., U.S. patent application Ser. No. 09/045,053, entitled TARGET ALLELES FOR ALLELE-SPECIFIC DRUGS, filed Mar. 19, 1998, which is hereby incorporated by reference in its entirety, including drawings.

The described approach and techniques are applicable to a variety of other diseases, conditions, and/or treatments and to genes associated with the etiology and pathogenesis of such other diseases and conditions and the efficacy and safety of such other treatments.

Useful variances for this invention can be described generally as variances which partition patients into two or more groups that respond differently to a therapy (a therapeutic intervention), regardless of the reason for the difference, and regardless of whether the reason for the difference is known.

### III. From Variance List to Clinical Trial: Identifying Genes and Gene Variances that Account for Variable Responses to Treatment

There are a variety of useful methods for identifying a subset of genes from a large set of candidate genes that should be prioritized for further investigation with respect to their influence on inter-individual variation in disease predisposition or response to a particular drug. These methods include for example, (1) searching the biomedical literature to identify genes relevant to a disease or the action of a drug, (2) screening the genes identified in step 1 for variances. A large set of exemplary variances are provided in U.S. patent application Ser. No. 09/689,506. Other methods include (3) using computational tools to predict the functional effects of variances in specific genes, (4) using in vitro or in vivo experiments to identify genes which may participate in the response to a drug or treatment, and to determine the variances which affect gene, RNA or protein function, and may therefore be important genetic variables affecting disease manifestations or drug response, and (5) retrospective or prospective clinical trials. Computational tools are described in U.S. patent application, Stanton et at., Ser. No., attorney docket number 241/034, filed Apr. 26, 1999, entitled GENE SEQUENCE VARIANCES WITH UTILITY IN DETERMINING THE TREATMENT OF DISEASE, and in Stanton et al., Ser. No. 09/419,705, filed Oct. 14, 1999, entitled VARIANCE SCANNING METHOD FOR IDENTIFYING GENE SEQUENCE VARIANCES, which are hereby incorporated by reference in their entireties, including drawings. Other methods are considered below in some detail.

(1) To begin, one preferably identifies, for a given treatment, a set of candidate genes that are likely to affect disease phenotype or drug response. This can be accomplished most efficiently by first assembling the relevant medical, pharmacological and biological data from available sources (e.g., public databases and publications). One skilled in the art can review the literature (textbooks, monographs, journal articles) and online sources (databases) to identify genes most relevant to the action of a specific drug or other treatment, particularly with respect to its utility for treating a specific disease, as this beneficially allows the set of genes to be analyzed ultimately in clinical trials to be reduced from an initial large set. Specific strategies for conducting such searches are described below. In some instances the literature may provide adequate information to select genes to be studied in a clinical trial, but in other cases additional experimental investigations of the sort described below will be preferable to maximize the likelihood that the salient genes and variances are moved forward into clinical studies. Specific genes relevant to understanding interpatient variation in response to treatments for major neurological and psychiatric diseases are listed in U.S. patent application Ser. No. 09/689,506. In preferred sets of genes for analysis of variable therapeutic response in specific diseases are highlighted. These genes are exemplary; they do not constitute a complete set of genes that may account for variation in clinical response. Experimental data are also useful in establishing a list of candidate genes, as described below.

(2) Having assembled a list of candidate genes generally the second step is to screen for variances in each candidate gene. Experimental and computational methods for variance detection are described in this invention, and tables of exemplary variances are provided in U.S. patent application Ser. No. xxxxx as well as methods for identifying additional variances and a written description of such possible additional variances in the cDNAs of genes that may affect drug action (see Stanton et al., application Ser. No. 09/300,747, filed Apr. 26, 1999, entitled GENE SEQUENCE VARIANCES WITH UTILITY IN DETERMINING THE TREATMENT OF DISEASE, incorporated in its entirety.

(3) Having identified variances in candidate genes the next step is to assess their likely contribution to clinical variation in patient response to therapy, preferably by using informatics-based approaches such as DNA and protein sequence analysis and protein modeling. The literature and informatics-based approaches provide the basis for prioritization of candidate genes, however it may in some cases be desirable to further narrow the list of candidate genes, or to measure experimentally the phenotype associated with specific variances or sets of variances (e.g. haplotypes).

(4) Thus, as a third step in candidate gene analysis, one skilled in the art may elect to perform in vitro or in vivo experiments to assess the functional importance of gene variances, using either biochemical or genetic tests. (Certain kinds of experiments—for example gene expression profiling and proteome analysis—may not only allow refinement of a candidate gene list but may also lead to identification of additional candidate genes.) Combination of two or all of the three above methods will provide sufficient information to narrow and prioritize the set of candidate genes and variances to a number that can be studied in a clinical trial with adequate statistical power.

(5) The fourth step is to design retrospective or prospective human clinical trials to test whether the identified allelic variance, variances, or haplotypes or combination thereof influence the efficacy or toxicity profiles for a given drug or other therapeutic intervention. It should be recognized that this fourth step is the crucial step in producing the type of data that would justify introducing a diagnostic test for at least one variance into clinical use. Thus while each of the above four steps are useful in particular instances of the invention, this final step is indispensable. Further guidance and examples of how to perform these five steps are provided below.

(6) A fifth (optional) step entails methods for using a genotyping test in the promotion and marketing of a treatment method. It is widely appreciated that there is a tendency in the pharmaceutical industry to develop many compounds for well established therapeutic targets. Examples include beta adrenergic blockers, hydroxymethylglutaryl (HMG) CoA reductase inhibitors (statins), dopamine D2 receptor antagonists and serotonin transporter inhibitors. Frequently the pharmacology of these compounds is quite similar in terms of efficacy and side effects. Therefore the marketing of one compound vs. other members of the class is a challenging problem for drug companies, and is reflected in the lesser success that late products typically achieve compared to the first and second approved products. It occurred to the inventors that genetic stratification can provide the basis for identifying a patient population with a superior response rate or improved safety to one member of a class of drugs, and that this information can be the basis for commercialization of that compound. Such a commercialization campaign can be directed at caregivers, particularly physicians, or at patients and their families, or both.

### 1. Identification of Candidate Genes Relevant to the Action of a Drug

Practice of this invention will often begin with identification of a specific pharmaceutical product, for example a drug, that would benefit from improved efficacy or reduced toxicity or both, and the recognition that pharmacogenetic investigations as described herein provide a basis for achieving such improved characteristics. The question then becomes which genes and variances, such as those provided in this application in U.S. patent application Ser. No. 09/689,506, would be most relevant to interpatient variation in response to the drug. As discussed above, the set of relevant genes includes both genes involved in the disease process and genes involved in the interaction of the patient and the treatment—for example genes involved in pharmacokinetic and pharmacodynamic action of a drug. The biological and biomedical literature and online databases provide useful guidance in selecting such genes. Specific guidance in the use of these resources is provided below.

### Review the literature and online sources

One way to find genes that affect response to a drug in a particular disease setting is to review the published literature and available online databases regarding the pathophysiology of the disease and the pharmacology of the drug. Literature or online sources can provide specific genes involved in the disease process or drug response, or describe biochemical pathways involving multiple genes, each of which may affect the disease process or drug response.

Alternatively, biochemical or pathological changes characteristic of the disease may be described; such information can be used by one skilled in the art to infer a set of genes that can account for the biochemical or pathologic changes. For example, to understand variation in response to a drug that modulates serotonin levels in a central nervous system (CNS) disorder associated with altered levels of serotonin one would preferably study, at a minimum, variances in genes responsible for serotonin biosynthesis, release from the cell, receptor binding, presynaptic reuptake, and degradation or metabolism. Genes responsible for each of these functions should be examined for variation that may account for interpatient differences in drug response or disease manifestations. As recognized by those skilled in the art, a comprehensive list of such genes can be obtained from textbooks, monographs and the literature.

There are several types of scientific information, described in some detail below, that are valuable for identifying a set of candidate genes to be investigated with respect to a specific disease and therapeutic intervention. First there is the medical literature, which provides basic information on disease pathophysiology and therapeutic interventions. A subset of this literature is devoted to specific description of pathologic conditions. Second there is the pharmacology literature, which will provide additional information on the mechanism of action of a drug (pharmacodynamics) as well as its principal routes of metabolic transformation (pharmacokinetics) and the responsible proteins. Third there is the biomedical literature (principally genetics, physiology, biochemistry and molecular biology), which provides more detailed information on metabolic pathways, protein structure and function and gene structure. Fourth, there are a variety of online databases that provide additional information on metabolic pathways, gene families, protein function and other subjects relevant to selecting a set of genes that are likely to affect the response to a treatment.

### Medical Literature

A good starting place for information on molecular pathophysiology of a specific disease is a general medical textbook such as Harrison's Principles of Internal Medicine, 14th edition, (2 Vol Set) by A. S. Fauci, E. Braunwald, K. J. Isselbacher, et al. (editors), McGraw Hill, 1997, or Cecil Textbook of Medicine (20th Ed) by R. L. Cecil, F. Plum and J. C. Bennett (Editors) W B Saunders Co., 1996. For pediatric diseases texts such as Nelson Textbook of Pediatrics (1 5th edition) by R. E. Behrman, R. M. Kliegman, A. M. Arvin and W. E. Nelson (Editors), W B Saunders Co., 1995 or Oski's Principles and Practice of Pediatrics (3rd Edition) by J. A. Mamillan & F. A. Oski Lippincott-Raven, 1999 are useful introductions. For obstetrical and gynecological disorders texts such as Williams Obstetrics (20th Ed) by F.G. Cunningham, N. F. Gant, P. C. McDonald et al. (Editors), Appleton & Lange, 1997 provide general information on disease pathophysiology. For psychiatric disorders texts such as the Comprehensive Textbook of Psychiatry, VI (2 Vols) by H. I. Kaplan and B. J. Sadock (Editors), Lippincott, Williams & Wilkins, 1995, or The American Psychiatric Press Textbook of Psychiatry (3rd edition) by R. E. Hales, S. C. Yudofsky and J. A. Talbott (Editors) Amer Psychiatric Press, 1999 provide an overview of disease nosology, pathophysiological mechanisms and treatment regimens.

In addition to these general texts, there are a variety of more specialized medical texts that provide greater detail about specific disorders which can be utilized in developing a list of candidate genes and variances relevant to interpatient variation in response to a treatment. For example, within the field of medicine there are standard textbooks for each of the subspecialties. Some specific examples include:

Heart Disease: A Textbook of Cardiovascular Medicine (2 Volume set) by E. Braunwald (Editor), W B Saunders Co., 1996.

Hurst's the Heart, Arteries and Veins (9th Ed) (2 Vol Set) by R. W. Alexander, R. C. Schlant, V. Fuster, W. Alexander and E. H. Sonnenblick (Editors) McGraw Hill, 1998.

Principles of Neurology (6th edition) by R. D. Adams, M. Victor (editors), and A. H. Ropper (Contributor), McGraw Hill, 1996.

Sleisenger & Fordtran's Gastrointestinal and Liver Disease: Pathophysiology, Diagnosis, Management (6th edition) by M. Feldman, B. F. Scharschmidt and M. Sleisenger (Editors), W B Saunders Co., 1997.

Textbook of Rheumatology (5th edition) by W. N. Kelley, S. Ruddy, E. D. Harris Jr. and C. B. Sledge (Editors) (2 volume set) W B Saunders Co., 1997.

Williams Textbook of Endocrinology (9th edition) by J. D. Wilson, D. W. Foster, H. M. Kronenberg and Larsen (Editors), W B Saunders Co., 1998.

Wintrobe's Clinical Hematology (10th Ed) by G. R. Lee, J. Foerster (Editor) and J. Lukens (Editors) (2 Volumes) Lippincott, Williams & Wilkins, 1998.

Cancer: Principles & Practice of Oncology (Sth edition) by V. T. Devita, S. A. Rosenberg and S. Hellman (editors), Lippincott-Raven Publishers, 1997.

Principles of Pulmonary Medicine (3rd edition) by S. E. Weinberger & J Fletcher (Editors), W B Saunders Co., 1998.

Diagnosis and Management of Renal Disease and Hypertension (2nd edition) by A. K. Mandal & J. C. Jennette (Editors), Carolina Academic Press, 1994.Massry & Glassock's Textbook of Nephrology (3rd edition) by S. G. Massry & R. J. Glassock (editors) Williams & Wilkins, 1995.

The Management of Pain by J. J. Bonica, Lea and Febiger, 1992 Ophthalmology by M. Yanoff & J. S. Duker, Mosby Year Book, 1998

Clinical Ophthalmology: A Systemic Approach by J. J. Kanski, Butterworth-Heineman, 1994.Essential Otolaryngology by J. K. Lee Appleton and Lange 1998.

In addition to these subspecialty texts there are many textbooks and monographs that concern more restricted disease areas, or specific diseases. Such books provide more extensive coverage of pathophysiologic mechanisms and therapeutic options. The number of such books is too great to provide examples for all but a few diseases, however one skilled in the art will be able to readily identify relevant texts. One simple way to search for relevant titles is to use the search engine of an online bookseller such as http://www.amazon.com or http://www.barnesandnoble.com using the disease or drug (or the group of diseases or drugs to which they belong) as search terms. For example a search for asthma would tun up titles such as Asthma: Basic Mechanisms and Clinical Management (3rd edition) by P. J. Barnes, I. W. Rodger and N. C. Thomson (Editors), Academic Press, 1998 and Airways and Vascular Remodelling in Asthma and Cardiovascular Disease Implications for Therapeutic Intervention, by C. Page & J. Black (Editors), Academic Press, 1994.

### Pathology Literature

In addition to medical texts there are texts that specifically address disease etiology and pathologic changes associated with disease. A good general pathology text is Robbins Pathologic Basis of Disease (6th edition) by R. S. Cotran, V. Kumar, T. Collins and S. L. Robbins, W B Saunders Co., 1998. Specialized pathology texts exist for each organ system and for specific diseases, similar to medical texts. These texts are useful sources of information for one skilled in the art for developing lists of genes that may account for some of the known pathologic changes in disease tissue. Exemplary texts are as follows:

Bone Marrow Pathology 2nd edition, by B. J. Bain, I. Lampert. & D. Clark, Blackwell Science, 1996

Atlas of Renal Pathology by F. G. Silva, W. B. Saunders, 1999.

Fundamentals of Toxicologic Pathology by W. M. Haschek and C. G. Rousseaux, Academic Press, 1997.

Gastrointestinal Pathology by P. Chandrasoma, Appleton and Lange, 1998.

Ophthalmic Pathology with Clinical Correlations by J. Sassani, Lippincott-Raven, 1997.

Pathology of Bone and Joint Disorders by F. McCarthy, F. J. Frassica and A. Ross, W. B. Saunders, 1998.

Pulmonary Pathology by M. A. Grippi, Lippicott-Raven, 1995.

Neuropathology by D. Ellison, L. Chimelli, B. Harding, S. Love& J. Lowe, Mosby Year Book, 1997.

Greenfield's Neuropatholgy 6th edition by J. G. Greenfield, P. L. Lantos & D. I. Graham, Edward Arnold, 1997.

### Pharmacology, Pharmacogenetics and Pharmacy Literature

There are also both general and specialized texts and monographs on pharmacology that provide data on pharmacokinetics and pharmacodynamics of drugs. The discussion of pharnacodynamics (mechanism of action of the drug) in such texts is often supported by a review of the biochemical pathway or pathways that are affected by the drug. Also, proteins related to the target protein are often listed; it is important to account for variation in such proteins as the related proteins may be involved in drug pharmacology. For example, there are 14 known serotonin receptors. Various pharmacological serotonin agonists or antagonists have different affinities for these different receptors. Variation in a specific receptor may affect the pharmacology not only of drugs targeted to that receptor, but also drugs that are principally agonists or antagonists of different receptors. Such compounds may produce different effects on two allelic forms of a non-targeted receptor; for example on variant form may bind the compound with higher affinity than the other, or a compound that is principally an antagonist for one allele may be a partial agonist for another allele. Thus genes encoding proteins structurally related to the target protein should be screened for variance in order to successfully realize the methods of the present invention. A good general pharmacology text is Goodman & Gilman's the Pharmacological Basis of Therapeutics (9th Ed) by J. G. Hardman, L. E. Limbird, P. B. Molinoff, R. W. Ruddon and A. G. Gilman (Editors) McGraw Hill, 1996. There are also texts that focus on the pharmacology of drugs for specific disease areas, or specific classes of drugs (e.g. natural products) or adverse drug interactions, among other subjects. Specific examples include:

The American Psychiatric Press Textbook of Psychopharmacology (2nd edition) by A. F. Schatzberg & C. B. Nemeroff (Editors), American Psychiatric Press, 1998. Essential Psychopharmacology: Neuroscientific Basis and Practical Applications by N. Muntner and S. M. Stahl, Cambridge Univ Press, 1996.

There are also texts on pharmacogenetics which are particularly useful for identifying genes which may contribute to variable pharmacokinetic response. In addition there are texts on some of the major xenobiotic metabolizing proteins, such as the cytochrome P450 genes.

Pharmacogenetics of Drug Metabolism (International Encyclopedia of Pharmacology and Therapeutics) by Werner Kalow (Editor) Pergamon Press, 1992.

Genetic Factors in Drug Therapy: Clinical and Molecular Pharmacogenetics by D. A Price Evans, Cambridge Univ Press, 1993.

Pharmacogenetics (Oxford Monographs on Medical Genetics, 32) by W. W. Weber, Oxford Univ Press, 1997.

Cytochrome P450: Structure, Mechanism, and Biochemistry by P. R. Ortiz de Montellano (Editor), Plenum Publishing Corp, 1995.

Appleton & Lange's Review of Pharmacy, 6th edition, (Appleton & Lange's Review Series) by G. D. Hall & B. S. Reiss, Appleton & Lange, 1997.

### Genetics, Biochemistry and Molecular Biology Literature

In addition to the medical, pathology, and pharmacology texts listed above there are several information sources that one skilled in the art will turn to for information on the genetic, physiologic, biochemical, and molecular biological aspects of the disease, disorder or condition or the effect of the therapeutic intervention on specific physiologic processes. The biomedical literature may include information on nonhuman organisms that is relevant to understanding the likely disease or pharmacological pathways in man.

Also provided below are illustrative texts which will aid in the identification of a pathway or pathways, and a gene or genes that may be relevant to interindividual variation in response to a therapy. Textbooks of biochemistry, genetics and physiology are often useful sources for such pathway information. In order to ascertain the appropriate methods to analyze the effects of an alleleic variance, variances, or haplotypes in vitro, one skilled in the art will review existing information on molecular biology, cell biology, genetics, biochemistry; and physiology. Such texts are useful sources for general and specific information on the genetic and biochemical processes involved in disease and in drug action, as well as experimental procedures that may be useful in performing in vitro research on an allelic variance, variances, or haplotye.

Texts on gene structure and function and RNA biochemistry will be useful in evaluating the consequences of variances that do not change the coding sequence (silent variances). Such variances may alter the interaction of RNA with proteins or other regulatory molecules affecting RNA processing, polyadenylation, or export.

### Molecular and Cellular Biology

Molecular Cell Biology by H. Lodish, D. Baltimore, A. Berk, L. Zipurksy & J. Darnell, W H Freeman & Co., 1995.

Essentials of Molecular Biology, D. Freifelder and MalacinskiJones and Bartlett, 1993.

Genes and Genomes: A Changing Perspective, M. Singer and P. Berg, 1991. University Science Books

Gene Structure and Expression, J. D. Hawkins, 1996. Cambridge University Press

Molecular Biology of the Cell, 2nd edition, B. Alberts et al., Garland Publishing, 1994.

### Molecular Genetics

The Metabolic and Molecular Bases of Inherited Disease by C. R. Scriver, A. L. Beaudet, W. S. Sly (Editors), 7th edition, McGraw Hill, 1995

Genetics and Molecular Biology, R. Schleif, 1994. 2nd edition, Johns Hopkins University Press

Genetics, P. J. Russell, 1996. 4th edition, Harper Collins

An Introduction to Genetic Analysis, Griffiths et al. 1993. 5th edition, W. H. Freeman and Company

Understanding Genetics: A molecular approach, Rothwell, 1993. Wiley-Liss

### General Biochemistry

Biochemistry, L. Stryer, 1995. W. H. Freeman and Company

Biochemistry, D. Voet and J. G. Voet, 1995. John Wiley and Sons

Principles of Biochemistry, A. L. Lehninger, D. L. Nelson, and M. M. Cox, 1993. Worth Publishers

Biochemistry, G. Zubay, 1998. Wm. C. Brown Communications

Biochemistry, C. K. Mathews and K. E. van Holde, 1990. Benjamin/Cummings

### Transcription

Eukaryotic Transcription Factors, D. S. Latchman, 1995. Academic Press

Eukaryotic Gene Transcription, S. Goodbourn (ed.), 1996. Oxford University Press.

Transcription Factors and DNA Replication, D. S. Pederson and N. H. Heintz, 1994. CRC Press/R. G. Landes Company

Transcriptional Regulation, S. L. McKnight and K. Yamamoto (eds.), 1992. 2 volumes, Cold Spring Harbor Laboratory Press

### RNA

Control of Messenger RNA Stability, J. Belasco and G. Brawerman (eds.), 1993. Academic Press

RNA-Protein Interactions, Nagai and Mattaj (eds.), 1994. Oxford University Press

mRNA Metabolism and Post-transcriptional Gene Regulation, Harford and Morris (eds.), 1997. Wiley-Liss

### Translation

Translational Control, J. W. B. Hershey, M. B. Mathews, and N. Sonenberg (eds.), 1995. Cold Spring Harbor Laboratory Press

### General Physiology

Textbook of Medical Physiology 9th Edtion by A. C. Guyton and J. E. Hall W. B. Saunders, 1997

Review of Medical Physiology, 18th Edition by W. F. Ganong, Appleton and Lange, 1997

### Online Databases

Those skilled in the art are familiar with how to search the biomedical literature, such as, e.g., libraries, online PubMed, abstract listings, and online mutation databases. One particularly useful resource is maintained at the web site of the National Center for Biotechnology Information (ncbi): http://www.ncbi.nlm.nih.gov/. From the ncbi site one can access Online Mendelian Inheritance in Man (OMIM). OMIM can be found at: http://www3.ncbi.nlm.nih.gov/Omim/searchomim.html. OMIM is a medically oriented database of genetic information with entries for thousands of genes. The OMIM record number is provided for many of the genes in In U.S. patent application serial no.xxxxx (see column 3), and constitutes an excellent entry point for identification of references that point to the broader literature. Another useful site at NCBI is the Entrez browser, located at http://www3.ncbi.nlm.nih.gov/Entrez/. One can search genomes, polynucleotides, proteins, 3D structures, taxonomy or the biomedical literature (PubMed) via the Entrez site. More generally links to a number of useful sites with biomedical or genetic data are maintained at sites such as Med Web at the Emory University Health Sciences Center Library: http://WWW.MedWeb.Emory.Bdu/MedWeb/; Riken, a Japanese web site at: http://www.rtc.riken.go jp/othersite.html with links to DNA sequence, structural, molecular biology, bioinformatics, and other databases; at the Oak Ridge National Laboratory web site: http://www.ornl.gov/hgmis/links.html; or at the Yahoo website of Diseases and Conditions: http://dir.yahoo.com/health/diseases_and_conditions/index.html. Each of the indicated web sites has additional useful links to other sites.

Another type of database with utility in selecting the genes on a biochemical pathway that may affect the response to a drug are databases that provide information on biochemical pathways. Examples of such databases include the Kyoto Encyclopedia of Genes and Genomes (KEGG), which can be found at: http://www.genome.adjp/kegg/kegg.html. This site has pictures of many biochemical pathways, as well as links to other metabolic databases such as the well known Boehringer Mannheim biochemical pathways charts: http://www.expasy.ch/cgi-bin/search-biochem-index. The metabolic charts at the latter site are comprehensive, and excellent starting points for working out the salient enzymes on any given pathway.

Each of the web sites mentioned above has links to other useful web sites, which in turn can lead to additional sites with useful information.Research Libraries

Those skilled in the art will often require information found only at large libraries. The National Library of Medicine (http://www.nlm.nih.gov/) is the largest medical library in the world and its catalogs can be searched online. Other libraries, such as university or medical school libraries are also useful to conduct searches. Biomedical books such as those referred to above can often be obtained from online bookstores as described above.

### Biomedical Literature

To obtain up to date information on drugs and their mechanism of action and biotransformation; disease pathophysiology; biochemical pathways relevant to drug action and disease pathophysiology; and genes that encode proteins relevant to drug action and disease one skilled in the art will consult the biomedical literature. A widely used, publically accessible web site for searching published journal articles is PubMed (http://www.ncbi.nlm.nih.gov/PubMed/). At this site, one can search for the most recent articles (within the last 1-2 months) or oler literature (back to 1966). Many Journals also have their own sites on the world wide web and can be searched online. For example see the IDEAL web site at: http://www.apnet.com/www/ap/aboutid.html. This site is an online library, featuring full text journals from Academic Press and selected journals from W.B. Saunders and Churchill Livingstone. The site provides access (for a fee) to nearly 2000 scientific, technical, and medical journals.

### Experimental methods for identification of genes involved in the action of a drug

There are a number of experimental methods for identifying genes and gene products that mediate or modulate the effects of a drug or other treatment. They encompass analyses of RNA and protein expression as well as methods for detecting protein-protein interactions and protein-ligand interactions. Two preferred experimental methods for identification of genes that may be involved in the action of a drug are (1) methods for measuring the expression levels of many mRNA transcripts in cells or organisms treated with the drug (2) methods for measuring the expression levels of many proteins in cells or organisms treated with the drug.

RNA transcripts or proteins that are substantially increased or decreased in drug treated cells or tissues relative to control cells or tissues are candidates for mediating the action of the drug. Preferably the level of an mRNA is at least 30% higher or lower in drug treated cells, more preferably at least 50% higher or lower, and most preferably two fold higher or lower than levels in non-drug treated control cells. The analysis of RNA levels can be performed on total RNA or on polyadenylated RNA selected by oligodT affinity. Further, RNA from different cell compartments can be analyzed independently—for example nuclear vs. cytoplasmic RNA. In addition to RNA levels, RNA kinetics can be examined, or the pool of RNAs currently being translated can be analyzed by isolation of RNA from polysomes. Other useful experimental methods include protein interaction methods such as the yeast two hybrid system and variants thereof which facilitate the detection of protein-protein interactions. Preferably one of the interacting proteins is the drug target or another protein strongly implicated in the action of the compound being assessed.

The pool of RNAs expressed in a cell is sometimes referred to as the transcriptome. Methods for measuring the transcriptome, or some part of it, are known in the art. A recent collection of articles summarizing some current methods appeared as a supplement to the journal Nature Genetics. (The Chipping Forecast. Nature Genetics supplement, volume 21, January 1999.) A preferred method for measuring expression levels of mRNAs is to spot PCR products corresponding to a large number of specific genes on a nylon membrane such as Hybond N Plus (Amersham-Pharmacia). Total cellular mRNA is then isolated, labelled by random oligonucleotide priming in the presence of a detectable label (e.g. alpha 33P labelled radionucleotides or dye labelled nucleotides), and hybridized with the filter containing the PCR products. The resulting signals can be analyzed by commercially available software, such as can be obtained from Clontech/Molecular Dynamics or Research Genetics, Inc.

Experiments have been described in model systems that demonstrate the utility of measuring changes in the transcriptome before before and after changing the growth conditions of cells, for example by changing the nutrient environment. The changes in gene expression help reveal the network of genes that mediate physiological responses to the altered growth condition. Similarly, the addition of a drug to the cellular or in vivo environment, followed by monitoring the changes in gene expression can aid in identification of gene networks that mediate pharmacological responses.

The pool of proteins expressed in a cell is sometimes referred to as the proteome. Studies of the proteome may include not only protein abundance but also protein subcellular localization and protein-protein interaction. Methods for measuring the proteome, or some part of it, are known in the art. One widely used method is to extract total cellular protein and separate it in two dimensions, for example first by size and then by isoelectric point. The resulting protein spots can be stained and quantitated, and individual spots can be excised and analyzed by mass spectrometry to provide definitive identification. The results can be compared from two or more cell lines or tissues, at least one of which has been treated with a drug. The differential up or down modulation of specific proteins in response to drug treatment may indicate their role in mediating the pharmacologic actions of the drug. Another way to identify the network of proteins that mediate the actions of a drug is to exploit methods for identifying interacting proteins. By starting with a protein known to be involved in the action of a drug—for example the drug target—one can use systems such as the yeast two hybrid system and variants thereof (known to those skilled in the art; see Ausubel et al., Current Protocols in Molecular Biology, op. cit.) to identify additional proteins in the network of proteins that mediate drug action. The genes encoding such proteins would be useful for screening for DNA sequence variances, which in turn may be useful for analysis of interpatient variation in response to treatments. For example, the protein 5-lipoxygenase (5LO) is an enzyme which is at the beginning of the leukotriene biosynthetic pathway and is a target for anti-inflammatory drugs used to treat asthma and other diseases. In order to detect proteins that interact with 5-lipoxygenase the two-hybrid system was recently used to isolate three different proteins, none previously known to interact with 5LO. (Provost et al., Interaction of 5-lipoxygenase with cellular proteins. Proc. Natl. Acad. Sci. U.S.A. 96: 1881-1885, 1999.) A recent collection of articles summarizing some current methods in proteomics appeared in the August 1998 issue of the journal Electrophoresis (volume 19, number 11). Other useful articles include: Blackstock W P, et al. Proteomics: quantitative and physical mapping of cellular proteins. Trends Biotechnol. 17 (3): p. 121-7, 1999, and Patton W. F., Proteome analysis II. Protein subcellular redistribution: linking physiology to genomics via the proteome and separation technologies involved. J Chromatogr B Biomed Sci App. 722(1-2):203-23. 1999.

Since many of these methods can also be used to assess whether specific polymorphisms are likely to have biological effects, they are also relevant in Section 3, below, concerning methods for assessing the likely contribution of variances in candidate genes to clinical variation in patient responses to therapy.

### 2. Screen for Variances in Genes that may be Related to Therapeutic Response

Having identified a set of genes that may affect response to a drug the next step is to screen the genes for variances that may account for interindividual variation in response to the drug. There are a variety of levels at which a gene can be screened for variances, and a variety of methods for variance screening. The two main levels of variance screening are genomic DNA screening and cDNA screening. Genomic variance detection may include screening the entire genomic segment spanning the gene from 2 kb to 10 kb upstream of the transcription start site to the polyadenylation site, or 2 to 10 kb beyond the polyadenylation site. Alternatively genomic variance detection may (for intron containing genes) include the exons and some region around them containing the splicing signals, for example, but not all of the intronic sequences. In addition to screening introns and exons for variances it is generally desirable to screen regulatory DNA sequences for variances. Promoter, enhancer, silencer and other regulatory elements have been described in human genes. The promoter is generally proximal to the transcription start site, although there may be several promoters and several transcription start sites. Enhancer, silencer and other regulatory elements may be intragenic or may lie outside the introns and exons, possibly at a considerable distance, such as 100 kb away. Variances in such sequences may affect basal gene expression or regulation of gene expression. In either case such variation may affect the response of an individual patient to a therapeutic intervention, for example a drug, as described in the examples. Thus in practicing the present invention it is useful to screen regulatory sequences as well as transcribed sequences, in order to identify variances that may affect gene transcription. Frequently the genomic sequence of a gene can be found in the sources above, particularly by searching GenBank or Medline (PubMed). The name of the gene can be entered at a site such as Entrez: http://www.ncbi.nlm.nih.gov/Entrez/nucleotide.html. Using the genomic sequence and information from the biomedical literature one skilled in the art can perform a variance detection procedure such as those described in examples 15, 16 and 17.

Variance detection is often first performed on the CDNA of a gene for several reasons. First, available data on functional sequence variances suggests that variances in the transcribed portion of a gene may be most likely to have functional consequences as they can affect the interaction of the transcript with a wide variety of cellular factors during the complex processes of RNA transcription, processing and translation, with consequent effects on RNA splicing, stability, translational efficiency or other processes. Second, as a practical matter the cDNA sequence of a gene is often available before the genomic structure is known, although the reverse will be true in the future as the sequence of the human genome is determined. Third, the cDNA is often compact compared to the genomic locus, and can be screened for variances with much less effort. If the genomic structure is not known then only the EDNA seqence can be scanned for variances. Methods for preparing cDNA are described in Example 7. Methods for variance detection on cDNA are described below and in the examples.

In general it is preferable to catalog genetic variation at the genomic DNA level because there are an increasing number of well documented instances of functionally important variances that lie outside of transcribed sequence. Also, to properly use optimal genetic methods to assess the contribution of a candidate gene to variation in a phenotype of interest it is desirable to understand the character of sequence variation in the candidate gene: what is the nature of linkage disequilibrium between different variances in the gene; are there sites of recombination within the gene; what is the extent of homoplasy in the gene (i.e. occurance of two variant sites that are identical by state but not identical by descent because the same variance arose at least twice in human evolutionary history on two different haplotypes); what are the different haplotypes and how can they be grouped to increase the power of genetic analysis?

Methods for variance screening have been described, including DNA sequencing. See for example: U.S. Pat. No. 5,698,400: Detection of mutation by resolvase cleavage; U.S. Pat. No. 5,217,863: Detection of mutations in nucleic acids; and U.S. Pat No. 5,750,335: Screening for genetic variation, as well as the examples and references cited therein for examples of useful variance detection procedures. Detailed variance detection procedures are also described in examples 15, 16 and 17. One skilled in the art will recognize that depending on the specific aims of a variance detection project (number of genes being screened, number of individuals being screened, total length of DNA being screened) one of the above cited methods may be preferable to the others, or yet another procedure may be optimal. A preferred method of variance detection is chain terminating DNA sequencing using dye labeled primers, cycle sequencing and software for assessing the quality of the DNA sequence as well as specialized software for calling heterozygotes. The use of such procedures has been described by Nickerson and colleagues. See for example: Rieder M. J., et al. Automating the identification of DNA variations using quality-based fluorescence re-sequencing: analysis of the human mitochondrial genome. Nucleic Acids Res. 26 (4):967-73, 1998, and: Nickerson D. A., et al. PolyPhred: automating the detection and genotyping of single nucleotide substitutions using fluorescence-based resequencing. Nucleic Acids Res. 25 (14):2745-51, 1997.Although the variances provided in U.S. patent application Ser. No. 09/689,506 consist principally of cDNA variances, it is an aspect of this invention that detection of genomic variances is also a useful method for identification of variances that may account for interpatient variation in response to a therapy.

Another important aspect of variance detection is the use of DNA from a panel of human subjects that represents a known population. For example, if the subjects are being screened for variances relevant to a specific drug development program it is desirable to include both subjects with the target disease and healthy subjects in the panel, because certain variances may occur at different frequencies in the healthy and disease populations and can only be reliably detected by screening both populations. Also, for example, if the drug development program is taking place in Japan, it is important to include Japanese individuals in the screening population. In general, it is always desirable to include subjects of known geographic, racial or ethnic identity in a variance screening experiment so the results can be interpreted appropriately for different patient populations, if necessary. Also, in order to select optimal sets of variances for genetic analysis of a gene locus it is desirable to know which variances have occurred recently—perhaps on multiple different chromosomes—and which are ancient. Inclusion of one or more apes or monkees in the variance screening panel is one way of gaining insight into the evolutionary history of variances. Chimpanzees are preferred subjects for inclusion in a variance screening panel.

### 3. Assess the Likely Contribution of Variances in Candidate Genes to Clinical Variation in Patient Responses to Therapy

Once a set of genes likely to affect disease pathophysiology or drug action has been identified, and those genes have been screened for variances, said variances (e.g., provided in Tables 3, and 4) can be assessed for their contribution to variation in the pharmacological or toxicological phenotypes of interest. Such studies are useful for reducing a large number of candidate variances to a smaller number of variances to be tested in clinical trials. There are several methods which can be used in the present invention for assessing the medical and pharmaceutical implications of a DNA sequence variance. They range from computational methods to in vitro and/or in vivo experimental methods, to prospective human clinical trials, and also include a variety of other laboratory and clinical measures that can provide evidence of the medical consequences of a variance. In general, human clinical trials constitute the highest standard of proof that a variance or set of variances is useful for selecting a method of treatment, however, computational and in vitro data, or retrospective analysis of human clinical data may provide strong evidence that a particular variance will affect response to a given therapy, often at lower cost and in less time than a prospective clinical trial. Moreover, at an early stage in the analysis when there are many possible hypotheses to explain interpatient variation in treatment response, the use of informatics-based approaches to evaluate the likely functional effects of specific variances is an efficient way to proceed.

Informatics-based approaches to the prediction of the likely functional effects of variances include DNA and protein sequence analysis (phylogenetic approaches and motif searching) and protein modeling (based on coordinates in the protein database, or pdb; see http://www.rcsb.org/pdb/). See, for example: Kawabata et al. The Protein Mutant Database. Nucleic Acids Research 27: 355-357, 1999; also available at: http://pmd.ddbj.nig.acjp. Such analyses can be performed quickly and inexpensively, and the results may allow selection of certain genes for more extensive in vitro or in vivo studies or for more variance detection or both.

The three dimensional structure of many medically and pharmaceutically important proteins, or homologs of such proteins in other species, or examples of domains present in such proteins, is known as a result of x-ray crystallography studies and, increasingly, nuclear magnetic resonance studies. Further, there are increasingly powerful tools for modeling the structure of proteins with unsolved structure, particularly if there is a related (homologous) protein with known structure. (For reviews see: Rost et al., Protein fold recognition by prediction-based threading, J. Mol. Biol. 270:471-480, 1997; Firestine et al., Threading your way to protein function, Chem. Biol. 3:779-783, 1996) There are also powerful methods for identifying conserved domains and vital amino acid residues of proteins of unknown structure by analysis of phylogenetic relationships. (Deleage et al., Protein structure prediction: Implications for the biologist, Biochimie 79:681-686, 1997; Taylor et al., Multiple protein structure alignment, Protein Sci. 3:1858-1870, 1994) These methods can permit the prediction of functionally important variances, either on the basis of structure or evolutionary conservation. For example, a crystal structure can reveal which amino acids comprise a small molecule binding site. The identification of a polymorphic amino acid variance in the topological neighborhood of such a site, and, in particular, the demonstration that at least one variant form of the protein has a variant amino acid which impinges on (or which may otherwise affect the chemical environment around) the small molecule binding pocket differently from another variant form, provides strong evidence that the variance may affect the function of the protein. From this it follows that the interaction of the protein with a treatment method, such an administered compound, will likely be variable between different patients. One skilled in the art will recognize that the application of computational tools to the identification of functionally consequential variances involves applying the knowledge and tools of medicinal chemistry and physiology to the analysis.

Phylogenetic approaches to understanding sequence variation are also useful. Thus if a sequence variance occurs at a nucleotide or encoded amino acid residue where there is usually little or no variation in homologs of the protein of interest from non-human species, particularly evolutionarily remote species, then the variance is more likely to affect function of the RNA or protein. Computational methods for phylogenetic analysis are known in the art, (see below for citations of some methods).

Computational methods are also useful for analyzing DNA polymorphisms in transcriptional regulatory sequences, including promoters and enhancers. One useful approach is to compare variances in potential or proven transcriptional regulatory sequences to a catalog of all known transcriptional regulatory sequences, including consensus binding domains for all transcription factor binding domains. See, for example, the databases cited in: Burks, C. Molecular Biology Database List. Nucleic Acids Research 27: 1-9, 1999, and links to useful databases on the internet at: http://www.oup.co.uk/nar/Volume_{—}27/issue_{—}01/summary/gkcO 05_gml.html. In particular see the Transcription Factor Database (Heinemeyer, T., et al. (1999) Expanding the TRANSFAC database towards an expert system of regulatory molecular mechanisms. Nucleic Acids Res. 27: 318-322, or on the internet at: http://193.175.244.40/TRANSFAC/index.html). Any sequence variances in transcriptional regulatory sequences can be assessed for their effects on mRNA levels using standard methods, either by making plasmid constructs with the different allelic forms of the sequence, transfecting them into cells and measuring the output of a reporter transcript, or by assays of cells with different endogenous alleles of variances. One example of a polymorphism in a transcriptional regulatory element that has a pharmacogenetic effect is described by Drazen et al. (1999) Pharmacogenetic association between ALOX5 promoter genotype and the response to anti-asthma treatment. Nature Genetics 22: 168-170. Drazen and co-workers found that a polymorphism in an Sp1-transcription factor binding domain, which varied among subjects from 3-6 tandem copies, accounted for varied expression levels of the 5-lipoxygenase gene when assayed in vitro in reporter construct assays. This effect would have been flagged by an informatics analysis that surveyed the 5-lipoxygenase candidate promoter region for transcriptional regulatory sequences (resulting in discovery of polymorphism in the Spl motif).

### 4. Perform in vitro or in vivo Experiments to Assess the Functional Importance of Gene Variances

There are two broad types of studies useful for assessing the likely functional importance of variances: (1) analysis of RNA or protein abundance and (2) analysis of functional differences in variant forms of a gene, mRNA or protein (e.g. variation in the catalytic properties or stability of an enzyme). Studies of functional differences may involve direct measurements of biochemical activity of different variant forms of an mRNA or protein, or may involve assaying the influence of a variance or variances on cell properties, including properties that can be measured in tissue culture or in vivo studies.

The selection of an appropriate experimental program for testing the medical consequences of a variance may differ depending on the nature of the variance, the gene, the disease and the type of treatment that the variance is likely to affect (e.g. treatment with a specific drug). For example, if there is evidence that a protein is involved in the pharmacologic action of a drug, then an in vitro or in vivo demonstration that an amino acid variance in the protein affects its biochemical activity, or is very likely to have such an effect, is strong evidence that the variance will have an effect on the pharmacology of the drug in patients, and therefore that patients with different variant forms of the gene may have different responses to the same dose of drug. Thus, the demonstration that a variance or variances in the gene encoding such a protein has an effect on mRNA or protein levels or function would constitute prima facie evidence that the variance has an effect on a therapeutic outcome. If the variance is silent with respect to protein coding information, or if it lies in a non-coding portion of the gene (e.g., a promoter or other regulatory sequence, an intron, or a 5′- or 3′-untranslated region), then the appropriate biochemical assay may be to assess mRNA abundance, half life, subcellular localization or translational efficiency (including, for example, the fraction of RNA bound to translational regulatory factors).

If, on the other hand, there is no substantial evidence that the protein encoded by a particular gene is relevant to drug pharmacology, but instead is a candidate gene due to its involvement in disease pathophysiology, or its differential expression in normal vs. disease tissue, then the optimal test of the therapeutic importance of a variance may be a clinical study addressing whether two patient groups distinguished on the basis of the variance respond differently to a therapeutic intervention.

In summary, if there is a plausible hypothesis regarding the effect of a protein on the action of a drug, then in vitro and in vivo approaches, including those described below, will often be useful to predict whether a given variance is therapeutically consequential. If, on the other hand, there is no evidence of such an effect, then the preferred test is often a clinical study of the impact of the variance on efficacy or toxicity (which requires no evidence or assumptions regarding the mechanism by which the variance may exert an effect on therapeutic response). Alternatively, a clinical study may focus on an accepted surrogate measure of efficacy or toxicity, in order to reduce the time and cost of the clinical study (e.g., the study may be a Phase I trial). However, given the expense and statistical constraints of clinical trials, it is preferable to limit clinical testing to variances for which there is at least some experimental or computational (i.e. predicted by phylogenetic analysis or modeling) evidence of a functional effect.

One can identify genetic determinants of drug response by studying the variation in drug response phenotypes among cell lines that have been typed for polymorphic markers. One then tests whether the phenotypic variation co-segregates with specific gene sequence variances or combinations of variances. Preferably the cell lines are derived from related individuals, because that approach allows the use of powerful genetic linkage analysis methods. Cells from unrelated individuals will also be useful, as described below, to show that specific variances have measurable effects even in subjects of widely varying genetic background. However, if there is an already established relationship between levels or functional activity of a protein and drug response then it is not necessary to treat cells with drug in order to produce data that strongly suggests a variance or variances in the gene encoding the protein affect treatment response. For example, if it is known that the level of expression of the drug target is an important determinant of treatment response, then demonstrating that level of the target, or of an mRNA encoding the target, vary among cell lines in a pattern that reveals co-segregation of expression levels with variances in the target, then that observation constitutes strong evidence of a pharmacogenetically important variance.

This method outlined above can be illustrated by considering thymidylate synthetase (TS), a primary target of the fluoropyrimidine drugs, including the direct-acting TS inhibitors such as raltitrexed, and some of the antifolate drugs. It is well documented that levels of TS mRNA or protein are inversely related to response to 5-fluorouracil/leukovorin treatment. Thus, low TS levels are associated with high response rates and vice versa. Hence identification of genetic determinants of TS mRNA or protein levels is likely to be of clinical significance. Thus observation that TS mRNA levels vary among cell lines, and that the variation segregates with the TS locus, indicates that a variance or variances at the TS locus affect mRNA levels, and constitutes good evidence that the variance or variances may be clinically significant. Similar arguments can be made for the targets of many other drugs.

One advantage of using cell lines from pedigrees is that it is not necessary to have identified a functionally important variance in order to determine that there must be such a variance. For example, consider a cellular drug response phenotype that is readily measured and that varies among cell lines. Again, an illuminating example might be levels of thymidylate synthetase mRNA in the translational pool 30 minutes after adding 5-fluorouracil, since 5-fluorouracil generally induces increased translation of thymidylate synthetase mRNA. A demonstration of Mendelian transmission of the drug response phenotype (here alteration of mRNA levels after drug administration) in cell lines from related individuals would constitute evidence of a genetic component to the drug response phenotype.

The expected pattern of segregation depends on making an assumption about the genetic model: recessive, dominant or co-dominant alleles will produce different proportions in the progeny of a cross. Since the location of the thymidylate synthetase (TS) gene is known (chromosome 18p) it can be readily determined whether polymorphic markers near the TS gene on 18p co-segregate with TS mRNA levels or any other TS related phenotype. Note that virtually any informative polymorphism in the vicinity of the TS gene—whether or not it is the functionally important polymorphism—will be sufficient to identify the TS gene as the causal gene. In some cases it will be desirable to confirm the results of genetic linkage or association studies using biochemical studies.

Alternatively, if levels of TS mRNA co-segregate with another chromosomal region then a variance in a different gene—perhaps a gene that encodes a transcription factor that is vital in regulating levels of TS transcription, or a gene that encodes an RNA binding protein that stabilizes TS mRNA—is primarily responsible for the effect. Based on the location and size of the chromosomal region that co-segregates with TS levels, and the known location of virtually all human genes, one can generate plausible hypotheses about the candidate genes likely to be responsible for any observed pattern of co-segregation. (Note that the size of the chromosomal region that co-segregates with TS levels is determined by the number of informative meioses that are analyzed in the linkage study; thus by analyzing more pedigrees, or by increasing the number of polymorphic markers in a specific chromosomal region until virtually all meioses are informative, one can improve the genetic resolution.)

It is also possible, even probable, that levels of TS mRNA are under the control of several loci on different chromosomes. There are well-tested methods for identifying loci responsible for a quantitative trait (quantitative trait loci, or QTLs). These methods are useful for mapping the location and magnitude of effect of two or more loci responsible for variation in an observed phenotype such as TS mRNA levels. Having identified genetic linkage between drug response and one or more loci in cell lines from one set of pedigrees, and having identified candidate genes at the loci that co-segregate with drug response, one can then perform genetic association studies in cell lines from unrelated individuals to determine whether the locus or loci identified by linkage also plays a significant role in cell lines derived from subjects with different genetic backgrounds.

The value of studying cell lines as surrogates for people is that experiments can be performed at a small fraction of the cost of clinical studies. The value of studying cell lines from related individuals is that genetic effects on drug response are likely to be much easier to identify when genetic background among the subjects is substantially similar. In particular, in cell lines from a pedigree it is known that only four parental alleles are segregating in the children, and that any two children are on average 50% genetically identical. In a more heterogeneous genetic background (i.e., cell lines from unrelated subjects) the effect of allelic variation at multiple genes that modulate the measured drug response phenotypes is more likely to create a nearly continuous distribution of responses, except in cases where the product of one gene accounts for most of the measured drug response phenotype.

Many cell lines have been derived from groups of related individuals, or pedigrees. A source of such cell lines is the Human Genetic Mutant Cell Repository, supported by the National Institute of General Medical Sciences (NIGMS) and housed at the Coriell Cell Repository, Camden, New Jersey. A directory of these cell lines is available on the world wide web: http://locus.umdnj.edu/nigms/. One preferred set of cell lines for pharmacogenetic studies, available from the Coriell Cell Repository, is the set of cell lines used by the Centre d'Etudes du Polymorphisme Humain (CEPH) consortium (Paris, France) to establish a detailed genetic map of man. See, for example: Gyapay, G., Morissette, J., Vignal, A., et al. (1994) The 1993-94 Genethon human genetic linkage map. Nature Genetics 7(2 Spec No):246-339. More current data on the CEPH genetic linkage map can be found on the world wide web at: http://landru.cephb.fr/cephdb/. Lymphoblastoid cell lines from 57 CEPH families are available from the Coriell Repository. In most cases the families consist of four grandparents, two parents and between four and twelve children.

The principal attraction of the CEPH cell lines for pharmacogenetic studies is that a detailed genetic map of 14,404 polymorphic markers has been established via an international effort (version 9.0 of the database was released in September 2000), and the map data are freely available for downloading via anonymous FTP on the world wide web at the following address: ftp://ftp.cephb.fr/pub/ceph_genotype_db. The current version of the database includes over 9,900 microsatellite markers, 56% of which are highly polymorphic. Further, according to information available at the web site, the mean observed heterozygote frequency of all the loci in version 9.0 is 0.70 (i.e. the heterozygote frequency for the average locus is 70% of the tested subjects). Also included in version 9.0 is data on 1,494 single nucleotide polymorphisms (SNPs) located throughout the human genome. Since the genotypes of thousands of polymorphic markers are known in most of the CEPH cell lines (not all markers were studied in all cell lines), one skilled in the art can determine the chromosomal location of any locus that controls a heritable trait in these cell lines, using software for linkage analysis such as the programs LINKAGE, CRIMAP or MAPMAKER. (See, for example: Lander, E. S., Green, P., Abrahamson, J., et al. (1987) MAPMAKER: an interactive computer package for constructing primary genetic linkage maps of experimental and natural populations. Genomics 1(2):174-81. See also: Ott, J. (1999) Analysis of Human Genetic Linkage. John Hopkins University Press, Baltimore, for a primer on the methods of genetic linkage analysis, and Terwilliger, J. and J. Ott (1994), Handbook of Human Linkage Analysis. John Hopkins University Press, Baltimore for a description of how to use linkage analysis software to analyze different types of data.)

Linkage between a variance or variances (multipoint linkage) and a phenotype is measured by a score called the LOD score, which is the logarithm of the ratio of the odds of the observed data occurring under the hypothesis of linkage to the odds of the observed data occurring under the hypothesis of no linkage (that is, a 50% chance of the genotype and phenotype assorting in the same way in each informative meiosis). LOD scores are calculated for specified values of theta, a measure of the genetic distance (recombination fraction) between the functionally important variance (read as the phenotype—e.g., mRNA levels of the gene encoding the drug target) and the variance which has been typed in the cell lines, and is being used to calculate the LOD score. As a rule, LOD scores over 3, indicating a 1000-fold greater likelihood of the hypothesis of linkage compared to the hypothesis of no linkage, are judged significant. Therefore, the LOD score for a genotype-phenotype linkage is preferably at least 3, more preferably 4 or more, still more preferably 5 or more and ideally 6 or greater (signifying one million fold greater likelihood that the observed data are explained by linkage). Given the density of markers in the CEPH map the value of theta is generally close to zero (that is, a variance can nearly always be found very close to the candidate gene). In the case of multipoint linkage analysis one can either use parametric techniques, which require specification of a mode of inheritance (dominant, co-dominant, recessive), or non-parametric techniques, which make no assumption about mode of inheritance.

As indicated above, one set of interesting Mendelian traits to study using the CEPH cell lines (or similar cell lines from pedigrees) and the genetic approach just described are drug response phenotypes. Consider, for example, a G protein coupled receptor that exists in two allelic forms that behave differently in the presence of a compound being developed for human clinical use (e.g., one form receptor binds the compound, an antagonist of the receptor, with higher affinity than the other form of the receptor). Methods for assaying G protein mediated signal transduction are well known in the art. By adding the compound, either at a fixed concentration or at a series of different concentrations, to a set of lymphoblastoid cell lines (which of course must express the G protein coupled receptor) derived from members of a family and measuring the signal produced by, for example, adding agonist in the presence of the drug it should be possible to determine whether the drug effect, however measured, segregates in the pedigree (represented by the cell lines), and in particular whether it segregates with the locus which encodes the G protein coupled receptor (GPCR). Detection of co-segregation of the drug response trait with the GPCR locus indicates the presence of functional variances in the GPCR. For example, consider two alleles of the receptor: if allele A produces a greater signal than allele B at a given concentration of the compound, and if one parent is an AB heterozygote while the other parent is a BB heterozygote then, assuming a co-dominant trait, the levels of signal in the children should be medium (in AB heterozygotes) or low (in BB homozygotes) compared to AA homozygotes in other families. The detection of such a pattern in cell lines of the family would constitute evidence that the G protein coupled receptor polymorphism was responsible for intersubject differences in response to the compound. (More generally, the detection of any discrete partitioning of responses in the data—high and low, or high medium and low—is suggestive of genetic control, with the genetic model to be inferred from the pattern of inheritance, and support for the hypothesis to come from the analysis of multiple families.)

It is not necessary to know the identity of the variant gene in advance (as in the G protein coupled receptor example just provided). The pattern of segregation of the drug response phenotype in the cell lines of the various members of the CEPH families can be compared to the pattern of segregation of the thousands of polymorphic markers already typed in the same cell lines. Those polymorphic markers that co-segregate with the drug response phenotype are candidates for marking the location of the locus or loci responsible for the drug response phenotype. By performing the same experiment in cell lines from multiple (e.g., from two up to 57 or more CEPH) families, the chromosome locations co-segregating with the drug response phenotype can be mapped to a high degree of resolution. Knowing (i) the chromosomal location of the gene (or genes) implicated by the linkage analysis, together with (ii) information about the location and function of genes in that chromosomal region (available from online databases, for example, those at the US National Center for Biotechnology Information (see http://www.ncbi.nlm.nih.gov/LocusLink/), and further (iii) knowing something of the pharmacology of the compound and consequently the metabolic and regulatory pathways likely to influence its action, should constrain the list of candidate genes likely to be responsible for the observed variation to a small number of genes. These genes (if there is more than one) can be systematically evaluated for phannacogenetic impact by identifying polymorphisms and testing whether they co-segregate with drug response phenotypes in the pedigrees, in new pedigrees, in cells from unrelated individuals, or in vivo in a population of non-related individuals, for example in a clinical trial.

Some drug response phenotypes may not behave as Mendelian traits, but may rather be continuous (quantitative) traits under the control of several genes. Variation at any of the relevant gene loci could affect drug response, often to different extents. Robust methods for mapping quantitative trait loci (QTL) are known in the art. For example, see: Shugart, Y. Y. and Goldgar, D. E. (1999) Multipoint genomic scanning for quantitative loci: effects of map density, sibship size and computational approach. Eur J Hum Genet 7(2):103-9. It is worth emphasizing that in the approach described (using the CEPH cell lines) there is no need for genotyping in order to map the drug response traits in the cell lines; the effort already expended to produce a human linkage map in the CEPH cell lines can be exploited.

Cell responses that could be usefully characterized by the above methods include, for example, the level of signaling in a pathway that mediates the response to a compound (as in the G protein coupled receptor assays where levels of a second messenger are measured), compound uptake, compound biotransformation (hydrolysis, oxidation, reduction, nitration, methylation, glyscosylation, glucuronidation and so forth), levels of endogenous small molecules such as folates, nucleosides, nucleotides, sugars, lipids, organic or inorganic ions, peptides and so forth that may be affected by a compound, levels of molecules involved in signal transduction such as diacylglycerol and phosphoinositol, proteins (including enzymes in biochemical pathways related to the action of the compound), levels of an inhibitory complex formed by a compound, and other molecules and assays known to those skilled in the art of pharmacology and assay development. For example, a study of the genetic basis of variation in response to the anti-neoplastic drug 5-fluorouracil might include measurement of cell uptake of radiolabelled 5-FU, conversion of 5-FU to inactive metabolites such as 5,6-dihydrofluorouridine or fluoro-beta alanine, conversion of 5-FU to active metabolites such as 5-fluorodeoxyuridine monophosphate, or 5fluorodeoxythymidine monophosphate, levels of thymidylate synthetase (an enzyme inhibited by 5-FU), levels of 5, 10 methylenetetrahydrofolate (a folate co-factor essential for 5-FU mediated inhibition of thymidylate synthetase) and the enzymes that produce it, or levels of nucleotide pools or the enzymes that produce them. All of the relevant transporters and enzymes are expressed in lymphoblastoid cells, even though 5-FU is not routinely used in the therapy of lymphoid malignancies.

However, a limitation of lymphoblastoid cell lines for the methods described above is that they are not suitable for all of the different types of assays one might wish to perform. One alternative is to use fibroblast cell lines, which, like lymphoblastoid cell lines, are already available from multiple different families through the Coriell Cell Repository. Fibroblasts are not available from the CEPH pedigrees, however a set of fibroblasts from pedigrees in the Coriell catalog could be genotyped at a set of highly polymorphic markers to produce a genetic map. Another approach is to treat lymphoblastoid cells with a procedure or agent that induces differentiation to a different cell type, such as an adipocye or a myocyte. For example, there are genes which effectively control differentiation programs (e.g., peroxisome proliferator activated receptor gamma [PPAR gamma] mediates adipocyte differentiation, myoD mediates myocyte differentiation). Introduction of such a gene into a cell line of one type can alter its differentiated state to another cell type. Alternatively, stimulation of the gene product of such a regulatory gene (e.g., treatment of cells with the PPAR gamma agonist troglitazone) can be used to induce differentiation to a different cell type. Such procedures are known in the art, and may be effectively applied to human lymphoblasts in order to create a cell type that expresses the gene(s) relevant to the pharmacogenetic project being undertaken.

In preferred embodiments of the above methods the cells used are from the CEPH pedigrees. Preferably at least one pedigree is studied, more preferably two pedigrees, still more preferably five pedigrees and most preferably eight pedigrees or more. The more pedigrees there are the more informative meioses and the higher the achievable LOD score. It is useful to perform a statistical power calculation before embarking on an analysis of cell lines, in order to determine how many pedigrees and cell lines should be studied to have acceptable power to detect an effect, making assumptions about the magnitude of the effect.

In another aspect, described below, the methods described above can be used to identify mRNAs that vary in levels between cell lines as a result of genetically controlled regulatory factors, such as, for example, polymorphisms in promoters that affect the binding or action of transcriptional regulatory factors. Such variation in mRNA levels may be responsible for intersubject variation in drug response.

In another aspect, it is useful to test for correlation between genetic variation and mRNA or protein levels in cell lines from unrelated individuals, using genetic association methods rather than linkage methods.

### Experimental Methods: Genomic DNA Analysis

Variances in DNA may affect the basal transcription or regulated transcription of a gene locus. Such variances may be located in any part of the gene but are most likely to be located in the promoter region, the first intron, or in DNA sequences flanking the 5′ or 3′ end of the gene, where enhancer or silencer elements may be located. Methods for analyzing transcription are well known to those skilled in the art and exemplary methods are briefly described above and in some of the texts cited elsewhere in this application. Transcriptional run off assay is one useful method. Detailed protocols can be found in texts such as: Current Protocols in Molecular Biology edited by: F. M. Ausubel, et al. John Wiley & Sons, Inc, 1999, or: Molecular Cloning: A Laboratory Manual by J. Sambrook, E. F. Fritsch and T Maniatis. 1989. 3 vols, 2nd edition, Cold Spring Harbor Laboratory Press

### Experimental Methods: RNA Analysis

RNA variances may affect a wide range of processes including RNA splicing, polyadenylation, capping, export from the nucleus, interaction with translation initiation, elongation or termination factors, or the ribosome, or interaction with cellular factors including regulatory proteins, or factors that may affect mRNA half life. However, the effect of most RNA sequence variances on RNA function, if any, should ultimately be measurable as an effect on RNA or protein levels—either basal levels or regulated levels or levels in some abnormal cell state, such as cells from patients with a disease. Therefore, one preferred method for assessing the effect of RNA variances on RNA function is to measure the levels of RNA produced by different alleles in one or more conditions of cell or tissue growth. Said measuring can be done by conventional methods such as Northern blots or RNAase protection assays (kits available from Ambion, Inc.), or by methods such as the Taqman assay (developed by the Applied Biosystems Division of the Perkin Elmer Corporation), or by using arrays of oligonucleotides or arrays of cDNAs attached to solid surfaces. Systems for arraying cDNAs are available commercially from companies such as Nanogen and General Scanning. Complete systems for gene expression analysis are available from companies such as Molecular Dynamics. For recent reviews of systems for high throughput RNA expression analysis see the supplement to volume 21 of Nature Genetics entitled “The Chipping Forecast”, especially articles beginning on pages 9, 15, 20 and 25.

Additional methods for analyzing the effect of variances on RNA include secondary structure probing, direct measurement of RNA half-life or turnover, and measuring RNA abundance in different cellular compartments (nucleus, cytoplasm, polysomes, etc.). Secondary structure can be determined by techniques such as enzymatic probing (using enzymes such as T1, T2 and S1 nuclease), chemical probing or RNAase H probing using oligonucleotides. Most RNA structural assays are performed in vitro, however some techniques can be performed on cell extracts or even in living cells, using fluorescence resonance energy transfer to monitor the state of RNA probe molecules.

In another aspect, the methods described above (relating to the use of cell lines from pedigrees to genetically map phenotypes amenable to analysis in tissue culture cells) can be used to identify mRNAs that vary in levels between individuals as a result of genetically controlled factors. Genetic factors include both cis-acting polymorphisms, such as might be present in promoters (e.g. polymorphisms that affect the binding or action of transcription factors) as well as trans-acting factors such as might be present in transcription factors (e.g., an amino acid polymorphism that affects the interaction of a transcription factor with a promoter element, or that might affect levels of the transcription factor itself). Variation in mRNA levels may contribute to intersubject variation in drug response, disease susceptibility or disease manifestations. (See above for example of promoter polymorphism in 5-lipoxygenase and its effect on response to anti-asthma medications.)

The methods for identifying mRNAs which vary in abundance as a consequence of genetic mechanisms are similar to those described above for drug response phenotypes. There are several kinds of experiments that would be useful in different settings.

First, consider a pharmacogenetic project in which there are one or more candidate genes that are known or believed to mediate the action of a drug. The questions one wishes to address include: is there variation in the levels or activity of the candidate genes; if so, is the variation in activity attributable to genetic variation (vs. environmental factors); and, optionally, is there evidence that the variation affects the way cells respond to drug. Second, consider a pharmacogenetic project in which relatively little is known about the molecular pharmacology of the compound being tested. The drug target may be known, but little else about the pharmacodynamic and pharmacokinetic behaviour of the compound is understood. in such a case it may be desirable to treat cells from related individuals with the compound and then measure gene expression as well as any drug response indices for which assays are available. The next step is to search for variation among the cell lines in patterns of gene expression, and specifically to identify genes whose expression is correlated with drug response indices. For example, one might find that most of the cell lines that have very low levels of a small molecule—the production of which was expected to be inhibited by the compound—also have high levels of expression of an mRNA that was not on the initial candidate gene list. Such a pattern of co-variation between the RNA levels and the drug response assay would identify the mRNA as a good candidate gene for explaining variation in response to the drug. The extreme version of this experiment is to use gene chip technology to simultaneously screen substantially all genes, to perform multiple assays (preferably real time, non-invasive assays) and to study cell lines from a large number of pedigrees in an attempt to identify virtually all of the significant associations between gene expression and inter-cell line variation in drug response. Clearly the genes whose expression is up or down modulated simply in response to exposure to the drug would be among the candidate genes one would monitor carefully for possible association with drug response.

The analysis of candidate genes could proceed as follows. First, by examining whether levels of an mRNA (say the mRNA for gene X) segregate with the locus encoding the mRNA in one or more pedigrees it is possible to infer whether there is a genetic component to the variation in mRNA levels. Second, if, by analyzing the CEPH genotype data using linkage methods it is possible to identify additional loci (beyond the locus which encodes gene X) that co-segregate with the mRNA expression levels (either increased or decreased) in the cell lines, then, as part of the output of the linkage analysis, one obtains the chromosomal location of the locus or loci that encodes a regulator of gene X mRNA levels. Third, by inspection of the genes known in the art to be located at the chromosomal region shown by linkage analysis to co-segregate with mRNA levels of gene X it should be possible to identify one or a few candidate genes that, on the basis of biological inference, are likely to account for the variation in mRNA levels (i.e., to be the regulators). These genes can then be definitively evaluated by identifying all variances (if not already known) and testing if they predict mRNA levels (or other phenotypes) in the pedigree cell lines, in cell lines from unrelated individuals, or in vivo. Fourth, the above analysis can be performed on cell lines subjected to various pharmacological or nutritional manipulations. For example, cell lines from one or more pedigrees can be treated with a drug, or deprived of an amino acid and mRNA levels measured at various times after treatment. Any variation in mRNA levels in response to the treatment, if the variation differs among individual cell lines, and if the different patterns of variation segregate in pedigrees, can be subjected to steps 1-3. Fifth, as indicated in the previous paragraph, this analysis can be performed at very large scale using arrays of gridded cDNAs, PCR products or oligonucleotides corresponding to an unlimited number of genes. In each experiment the RNA from the pedigree cell lines (drug treated or not) is isolated, labeled using standard methods and hybridized to the grids containing the nucleic acids corresponding to the genes being investigated. Current commercial methods permit up to 400,000 oligonucleotides (more than the total number of human genes) to be queried in one experiment, although lower density formats are also well suited to the methods described. A preferred density of oligonucleotides or PCR products is at least 1000 per glass slide, more preferably 2000 per slide. Thus, in a comparatively modest number of experiments the entire transcript population of lymphoblasts (probably <25,000 unique transcripts) can be queried for genetically controlled variation in MRNA abundance. Other types of cell lines can be subjected to similar analysis.

In another embodiment one can use mRNAexpression profiling data from cell lines from pedigrees to identify substantially all loci that exhibit population variation in mRNA abundance that is determined by genetic variation at the locus. The steps are to (i) perform gene expression studies of a large number of cell lines from pedigrees, and (ii) for all mRNAs that exhibit variation, test for linkage with the locus that encodes the mRNA. This approach has the advantage of being a one-step method to identify a substantial fraction of all genes that exhibit variation due to DNA polymorphism.

In general, the variation in mRNA levels due to gene polymorphisms is likely to be of small magnitude (generally two-fold differences or less are expected). Therefore a key aspect of experimental systems used to measure mRNA levels is their accuracy. Preferably a system capable of resolving mRNAs that differ in abundance (measured in molecules per cell, or relative to a standard such as total mRNA or one or more specific RNAs such as actin or clathrin or glucose-6-phosphare dehydrogenase) is sufficiently sensitive to detect differences as small as 50%, more preferably as small as 30%, and most preferably as small as 20%.

There are 757 individuals in the 57 CEPH cell lines. Thus all the CEPH cell lines could fit in eight 96 well microtiter plates. Microtiter plates provide a convenient format for growing cells and for performing cell manipulations, such as those described above, using multichannel pipettes or automated pipetting robots. By growing cells in large volume flasks, counting them (by hemocytometer or Coulter counter or other means) and then aliquoting them robotically to 96 well plates it is possible to assure that each well has nearly the same number of cells. A large number of plates can be prepared in this way and then stored frozen in appropriate medium until needed for experiments.

### Experimental Methods: Protein Analysis

There are a variety of experimental methods for investigating the effect of an amino acid variance on response of a patient to a treatment. The preferred method will depend on the availability of cells expressing a particular protein, and the feasibility of a cell-based assay vs. assays on cell extracts, on proteins produced in a foreign host, or on proteins prepared by in vitro translation.

For example, the methods and systems listed below can be utilized to demonstrate differential expression, stability and/or activity of different variant forms of a protein, or in phenotype/genotype correlations in a model system.

For the determination of protein levels or protein activity a variety of techniques are available. The in vitro protein activity can be determined by transcription or translation in bacteria, yeast, baculovirus, COS cells (transient), Chinese Hamster Ovary (CHO) cells, or studied directly in human cells, or other cell systems can be used. Further, one can perform pulse chase experiments to determine if there are changes in protein stability (half-life).

One skilled in the art can construct cell-based assays of protein function, and then perform the assays in cells with different genotypes or haplotypes. For example, identification of cells with different genotypes, e.g., cell lines established from families and subsequent determination of relevant protein phenotypes (e.g., expression levels, post translational modifications, activity assays) may be performed using standard methods.

Assays of protein levels or function can also be performed on cell lines (or extracts from cell lines) derived from pedigrees in order to determine whether there is a genetic component to variation in protein levels or function. The experimental analysis is as above for RNAs, except the assays are different. Experiments can be performed on naive cells or on cells subjected to various treatments, including pharmacological treatments.

In another approach to the study of amino acid variances one can express genes corresponding to different alleles in experimental organisms and examine effects on disease phenotype (if relevant in the animal model), or on response to the presence of a compound. Such experiments may be performed in animals that have disrupted copies of the homologous gene (e.g. gene knockout animals engineered to be deficient in a target gene), or variant forms of the human gene may be introduced into germ cells by transgenic methods, or a combination of approaches may be used. To create animal strains with targeted gene disruptions a DNA construct is created (using DNA sequence information from the host animal) that will undergo homologous recombination when inserted into the nucleus of an embryonic stem cell. The targeted gene is effectively inactivated due to the insertion of non-natural sequence—for example a translation stop codon or a marker gene sequence that interrupts the reading frame. Well-known PCR based methods are then used to screen for those cells in which the desired homologous recombination event has occurred. Gene knockouts can be accomplished in worms, drosophila, mice or other organisms. Once the knockout cells are created (in whatever species) the candidate therapeutic intervention can be administered to the animal and pharmacological or biological responses measured, including gene expression levels. If variant forms of the gene are useful in explaining interpatient variation in response to the compound in man, then complete absence of the gene in an experimental organism should have a major effect on drug response. As a next step various human forms of the gene can be introduced into the knockout organism (a technique sometimes referred to as a knock-in). Again, pharmacological studies can be performed to assess the impact of different human variances on drug response. Methods relevant to the experimental approaches described above can be found in the following exemplary texts:

### General Molecular Biology Methods

Molecular Biology: A project approach, S. J. Karcher, Fall 1995. Academic Press

DNA Cloning: A Practical Approach, D. M. Glover and B. D. Hayes (eds). 1995. IRL/Oxford University Press. Vol. 1—Core Techniques; Vol 2—Expression Systems; Vol. 3—Complex Genomes; Vol. 4—Mammalian Systems.

Short Protocols in Molecular Biology, Ausubel et al. October 1995. 3rd edition, John Wiley and Sons

Current Protocols in Molecular Biology Edited by: F. M. Ausubel, R. Brent, R. E. Kingston, D. D. Moore, J. G. Seidman, K. Struhl, (Series Editior: V. B. Chanda), 1988

Molecular Cloning: A laboratory manual, J. Sambrook, E. F. Fritsch. 1989. 3 vols, 2nd edition, Cold Spring Harbor Laboratory Press

### Polymerase Chain Reaction (PCR)

PCR Primer: A laboratory manual, C. W. Diffenbach and G. S. Dveksler (eds.). 1995. Cold Spring Harbor Laboratory Press.

The Polymerase Chain Reaction, K. B. Mullis et al. (eds.), 1994. Birkhauser

PCR Strategies, M. A. Innis, D. H. Gelf, and J. J. Sninsky (eds.), 1995. Academic Press

### General Procedures for Discipline Specific Studies

Current Protocols in Neuroscience Edited by: J Crawley, C. Gerfen, R. McKay, M. Rogawski, D. Sibley, P. Skolnick, (Series Editor: G. Taylor), 1997.

Current Protocols in Pharmacology Edited by: S. J. Enna/M. Williams, J. W. Ferkany, T. Kenakin, R. E. Porsolt, J. P. Sullivan, (Series Editor: G. Taylor),1998.

Current Protocols in Protein Science Edited by: J. E. Coligan, B. M. Dunn, H. L. Ploegh, D. W. Speicher, P. T. Wingfield, (Series Editor: Virginia Benson Chanda), 1995.

Current Protocols in Cell Biology Edited by: J. S. Bonifacino, M. Dasso, J. Lippincott-Schwartz, J. B. Harford, K. M. Yamada, (Series Editor: K. Morgan) 1999.

Current Protocols in Cytometry Managing Editor: J. P. Robinson, Z. Darzynkiewicz (ed)/P. Dean (ed), A. Orfao (ed), P. Rabinovitch (ed), C. Stewart (ed), H. Tanke (ed), L. Wheeless (ed), (Series Editor: J. Paul Robinson), 1997.

Current Protocols in Human Genetics Edited by: N. C. Dracopoli, J. L. Haines, B. R. Korf, et al., (Series Editor: A. Boyle), 1994.

Current Protocols in Immunology Edited by: J. E. Coligan, A. M. Kruisbeek, D. H. Margulies, E. M. Shevach, W. Strober, (Series Editor: R. Coico), 1991.

### IV. Clinical Trials

A clinical trial is the definitive test of the utility of a variance or variances for the selection of optimal therapy. A clinical trial in which an interaction of gene variances and clinical outcomes (desired or undesired) is explored will be referred to herein as a “pharmacogenetic clinical trial”. Pharmacogenetic clinical trials require no knowledge of the biological function of the gene containing the variance or variances to be assessed, nor any knowledge of how the therapeutic intervention to be assessed works at a biochemical level. The pharmacogenetics effects of a variance can be addressed at a purely statistical level: either a particular variance or set of variances is consistently associated with a significant difference in a salient drug response parameter (e.g. response rate, effective dose, side effect rate, etc.) or not. On the other hand, if there is information about either the biochemical basis of a therapeutic intervention or the biochemical effects of a variance, then a pharmacogenetic clinical trial can be designed to test a specific hypothesis. In preferred embodiments of the methods of this application the mechanism of action of the compound to be genetically analyzed is at least partially understood.

Methods for performing clinical trials are well known in the art. (see e.g. Guide to Clinical Trials by Bert Spilker, Raven Press, 1991; The Randomized Clinical Trial and Therapeutic Decisions by Niels Tygstrup (Editor), Marcel Dekker; Recent Advances in Clinical Trial Design and Analysis (Cancer Treatment and Research, Ctar 75) by Peter F. Thall (Editor) Kluwer Academic Pub, 1995. Clinical Trials: A Methodologic Perspective by Steven Piantadosi, Wiley Series in Probability and Statistics, 1997). However, performing a clinical trial to test the genetic contribution to interpatient variation in drug response entails additional design considerations, including (i) defining the genetic hypothesis or hypotheses, (ii) devising an analytical strategy for testing the hypothesis, including determination of how many patients will need to be enrolled to have adequate statistical power to measure an effect of a specified magnitude (power analysis), (iii) definition of any primary or secondary genetic endpoints, and (iv) definition of methods of statistical genetic analysis, as well as other aspects. In the outline below some of the major types of genetic hypothesis testing, power analysis and statistical testing and their application in different stages of the drug development process are reviewed. One skilled in the art will recognize that certain of the methods will be best suited to specific clinical situations, and that additional methods are known and can be used in particular instances.

### V. Variance Identification and Use

### A. Initial Identification of Variances in Genes

Selection of Population Size and Composition

Prior to testing to identify the presence of sequence variances in a particular gene or genes, it is useful to understand how many individuals should be screened to provide confidence that most or nearly all pharmacogenetically relevant variances will be found. The answer depends on the frequencies of the phenotypes of interest and what assumptions we make about heterogeneity and magnitude of genetic effects. Prior to testing to identify the presence of sequence variances in a particular gene or genes, it is useful to understand how many individuals should be screened to provide confidence that most or nearly all pharmacogenetically relevant variances will be found. The answer depends on the frequencies of the phenotypes of interest and what assumptions we make about heterogeneity and magnitude of genetic effects. At the beginning we only know phenotype frequencies (e.g. responders vs. nonresponders, frequency of various side effects, etc.).

The most conservative assumption (resulting in the lowest estimate of allele frequency, and consequently the largest suggested screening population) is (i) that the phenotype (e.g. toxicity or efficacy) is multifactorial (i.e. can be caused by two or more variances or combinations of variances), (ii) that the variance of interest has a high degree of penetrance (i.e. is consistently associated with the phenotype), and (iii) that the mode of transmission is Mendelian dominant. Consider a pharmacogenetic study designed to identify predictors of efficacy for a compound that produces a 15% response rate in a nonstratified population. If half the response is sustantially attributable to a given variance, and the variance is consistently associated with a positive response (in 80% of cases) and the variance need only be present in one copy to produce a positive result then ˜10% of the subjects are likely heterozygotes for the variance that produces the response. The Hardy-Weinberg equation can be used to infer an allele frequency in the range of 5% from these assumptions (given allele frequencies of 5%/95% then: 2×0.05×0.95=0.095, or 9.5% heterozygotes are expected, and 0.05×0.05=0.0025, or 0.25% homozygotes are expected. They sum to 9.5% +0.25% =9.75% likely responders, 80% of whom, or 7.6%, are likely real responders due to presence of the positive response allele. Thus about half of the 15% responders are accounted for.). From the Table it can be seen that, in order to have a 99% chance of detecting an allele present at a frequency of 5% nearly 50 subjects should be screened for variances, assuming that the variances occur in the screening population at the same frequency as they occur in the patient population. Similar analyses can be performed for other assumptions regarding likely magnitude of effect, penetrance and mode of genetic transmission.

At the beginning we only know phenotype frequencies (e.g. responders vs. nonresponders, frequency of various side effects, etc.). As an example, the occurrence of serious 5-FU/FA toxicity—e.g. toxicity requiring hospitalization is often >10%. The occurrence of life threatening toxicity is in the 1-3% range (Buroker et al. 1994). The occurrence of complete remissions is on the order of 2-8%. The lowest frequency phenotypes are thus on the order of ˜2%. If we assume that (i) homogeneous genetic effects are responsible for half the phenotypes of interest and (ii) for the most part the extreme phenotypes represent recessive genotypes, then we need to detect alleles that will be present at ˜10% frequency (0.1×0.1=0.01, or 1% frequency of homozygotes) if the population is at Hardy-Weinberg equilibrium. To have a ˜99% chance of identifying such alleles would require searching a population of 22 individuals (see Table below). If the major phenotypes are associated with heterozygous genotypes then we need to detect alleles present at ˜0.5% frequency (2×0.005×0.995=0.00995, or ˜1% frequency of heterozygotes). A 99% chance of detecting such alleles would require ˜40 individuals (Table below). Given the heterogeneity of the North American population we cannot assume that all genotypes are present in Hardy-Weinberg proportions, therefore a substantial oversampling may be done to increase the chances of detecting relevant variances: For our initial screening, usually, 62 individuals of known race/ethnicity are screened for variance. Variance detection studies can be extended to outliers for the phenotypes of interest to cover the possibility that important variances were missed in the normal population screening.

| Allele | Number of subjects genotyped | | | | | | | |
|---|---|---|---|---|---|---|---|---|
| frequencies | n = 5 | n = 10 | n = 15 | n = 20 | n = 25 | n = 30 | n = 35 | n = 50 |
| p = .99, | 9.56 | 18.21 | 26.03 | 33.10 | 39.50 | 45.28 | 50.52 | 63.40 |
| p = .97, | 26.26 | 45.62 | 59.90 | 70.43 | 78.19 | 83.92 | 88.14 | 95.24 |
| p = .95, | 40.13 | 64.15 | 78.53 | 87.15 | 92.30 | 95.39 | 97.24 | 99.65 |
| p = .93, | 51.60 | 76.58 | 88.66 | 94.51 | 97.34 | 98.71 | 99.38 | 99.93 |
| p = .9, q = | 65.13 | 87.84 | 95.76 | 98.52 | 99.48 | 99.82 | 99.94 | >99.9 |
| p = .8, q = | 89.26 | 98.84 | 99.88 | 99.99 | >99.9 | >99.9 | >99.9 | >99.9 |
| p = .7, q = | 97.17 | 99.92 | 99.99 | >99.9 | >99.9 | >99.9 | >99.9 | >99.9 |

### Likelihood of Detecting Polymorphism in a Population as a Function of Allele Frequency & Number of Individuals Genotyped

The table above shows the probability (expressed as percent) of detecting both alleles (i.e. detecting heterozygotes) at a biallelic locus as a function of (i) the allele frequencies and (ii) the number of individuals genotyped. The chances of detecting heterozygotes increases as the frequencies of the two alleles approach 0.5 (down a column), and as the number of individuals genotyped increases (to the right along a row). The numbers in the table are given by the formula: 1−(p)2n−(q)2n. Allele frequencies are designated p and q and the number of individuals tested is designated n. (Since humans are diploid, the number of alleles tested is twice the number of individuals, or 2n.)

While it is preferable that numbers of individuals, or independent sequence samples, are screened to identify variances in a gene, it is also very beneficial to identify variances using smaller numbers of individuals or sequence samples. For example, even a comparison between the sequences of two samples or individuals can reveal sequence variances between them. Preferably, 5, 10, or more samples or individuals are screened.

### Source of Nucleic Acid Samples

Nucleic acid samples, for example for use in variance identification, can be obtained from a variety of sources as known to those skilled in the art, or can be obtained from genomic or cDNA sources by known methods. For example, the Coriell Cell Repository (Camden, N.J.) maintains over 6,000 human cell cultures, mostly fibroblast and lymphoblast cell lines comprising the NIGMS Human Genetic Mutant Cell Repository. A catalog (http://locus.umdnj.edu/nigms) provides racial or ethnic identifiers for many of the cell lines. It is preferable to perform polymorphism discovery on a population that mimics the population to be evaluated in a clinical trial, both in terms of racial/ethnic/geographic background and in terms of disease status. Otherwise, it is generally preferable to include a broad population sample including, for example, (for trials in the United States): Caucasians of Northern, Central and Southern European origin, Africans or African-Americans, Hispanics or Mexicans, Chinese, Japanese, American Indian, East Indian, Arabs and Koreans.

### Source of Human DNA, RNA and cDNA Samples

PCR based screening for DNA polymorphism can be carried out using either genomic DNA or cDNA produced from mRNA. For many genes, only cDNA sequences have been published, therefore the analysis of those genes is, at least initially, at the cDNA level since the determination of intron-exon boundaries and the isolation of flanking sequences is a laborious process. However, screening genomic DNA has the advantage that variances can be identified in promoter, intron and flanking regions. Such variances may be biologically relevant. Therefore preferably, when variance analysis of patients with outlier responses is performed, analysis of selected loci at the genomic level is also performed. Such analysis would be contingent on the availability of a genomic sequence or intron-exon boundary sequences, and would also depend on the anticipated biological importance of the gene in connection with the particular response.

When cDNA is to be analyzed it is very beneficial to establish a tissue source in which the genes of interest are expressed at sufficient levels that cDNA can be readily produced by RT-PCR. Preliminary PCR optimization efforts for 19 of the 29 genes in Table 2 reveal that all 19 can be amplified from lymphoblastoid cell mRNA. The 7 untested genes belong on the same pathways and are expected to also be PCR amplifiable.

### PCR Optimization

Primers for amplifying a particular sequence can be designed by methods known to those skilled in the art, including by the use of computer programs such as the PRIMER software available from Whitehead Institute/MIT Genome Center. In some cases it is preferable to optimize the amplification process according to parameters and methods known to those skilled in the art; optimization of PCR reactions based on a limited array of temperature, buffer and primer concentration conditions is utilized. New primers are obtained if optimization fails with a particular primer set.

### Variance Detection Using T4 Endonuclease VII Mismatch Cleavage Method

Any of a variety of different methods for detecting variances in a particular gene can be utilized, such as those described in the patents and applications cited in section A above. An exemplary method is a T4 EndoVII method. The enzyme T4 endonuclease VII (T4E7) is derived from the bacteriophage T4. T4E7 specifically cleaves heteroduplex DNA containing single base mismatches, deletions or insertions. The site of cleavage is 1 to 6 nucleotides 3′ of the mismatch. This activity has been exploited to develop a general method for detecting DNA sequence variances (Youil et al. 1995; Mashal and Sklar, 1995). A quality controlled T4E7 variance detection procedure based on the T4E7 patent of R. G. H. Cotton and co-workers. (Del Tito et al., in press) is preferably utilized. T4E7 has the advantages of being rapid, inexpensive, sensitive and selective. Further, since the enzyme pinpoints the site of sequence variation, sequencing effort can be confined to a 25 -30 nucleotide segment.

The major steps in identifying sequence variations in candidate genes using T4E7 are: (1) PCR amplify 400-600 bp segments from a panel of DNA samples; (2) mix a fluorescently-labeled probe DNA with the sample DNA; (3) heat and cool the samples to allow the formation of heteroduplexes; (4) add T4E7 enzyme to the samples and incubate for 30 minutes at 37oC, during which cleavage occurs at sequence variance mismatches; (5) run the samples on an ABI 377 sequencing apparatus to identify cleavage bands, which indicate the presence and location of variances in the sequence; (6) a subset of PCR fragments showing cleavage are sequenced to identify the exact location and identity of each variance.

The T4E7 Variance Imaging procedure has been used to screen particular genes. The efficiency of the T4E7 enzyme to recognize and cleave at all mismatches has been tested and reported in the literature. One group reported detection of 81 of 81 known mutations (Youil et al. 1995) while another group reported detection of 16 of 17 known mutations (Mashal and Sklar, 1995). Thus, the T4E7 method provides highly efficient variance detection.

### DNA sequencing

A subset of the samples containing each unique T4E7 cleavage site is selected for sequencing. DNA sequencing can, for example, be performed on ABI 377 automated DNA sequencers using BigDye chemistry and cycle sequencing. Analysis of the sequencing runs will be limited to the 30-40 bases pinpointed by the T4E7 procedure as containing the variance. This provides the rapid identification of the altered base or bases.

In some cases, the presence of variances can be inferred from published articles which describe Restriction Fragment Length Polymorphisms (RFLP). The sequence variances or polymorphisms creating those RFLPs can be readily determined using convention techniques, for example in the following manner. If the RFLP was initially discovered by the hybridization of a cDNA, then the molecular sequence of the RFLP can be determined by restricting the cDNA probe into fragments and separately hybridizing to a Southern blot consisting of the restriction digestion with the enzyme which reveals the polymorphic site, identifying the sub-fragment which hybridizes to the polymorphic restriction fragment, obtaining a genomic clone of the gene (e.g., from commercial services such as Genome Systems (Saint Louis, Mo.) or Research Genetics (Alabama) which will provide appropriate genomic clones on receipt of appropriate primer pairs). Using the genomic clone, restrict the genomic clone with the restriction enzyme which revealed the polymorphism and isolate the fragment which contains the polymorphism, e.g., identifying by hybridization to the cDNA which detected the polymorphism. The fragment is then sequenced across the polymorphic site. A copy of the other allele can be obtained by PCT from addition samples.

### Variance Detection Using Sequence Scanning

In addition to the physical methods, e.g., those described above and others known to those skilled in the art (see, e.g., Housman, U.S. Pat. No. 5,702,890; Housman et al., U.S. patent application Ser. No. 09/045,053), variances can be detected using computational methods, involving computer comparison of sequences from two or more different biological sources, which can be obtained in various ways, for example from public sequence databases. The term “variance scanning” refers to a process of identifying sequence variances using computer-based comparison and analysis of multiple representations of at least a portion of one or more genes. Computational variance detection involves a process to distinguish true variances from sequencing errors or other artifacts, and thus does not require perfectly accurate sequences. Such scanning can be performed in a variety of ways, preferably, for example, as described in Stanton et al., filed Oct. 14, 1999, Ser. No. 09/419,705, attorney docket number 246/128.

While the utilization of complete cDNA sequences is highly preferred, it is also possible to utilize genomic sequences. Such analysis may be desired where the detection of variances in or near splice sites is sought. Such sequences may represent full or partial genomic DNA sequences for a gene or genes. Also, as previously indicated, partial cDNA sequences can also be utilized although this is less preferred. As described below, the variance scanning analysis can simply utilize sequence overlap regions, even from partial sequences. Also, while the present description is provided by reference to DNA, e.g., cDNA, some sequences may be provided as RNA sequences, e.g., mRNA sequences. Such RNA sequences may be converted to the corresponding DNA sequences, or the analysis may use the RNA sequences directly.

### B. Determination of Presence or Absence of Known Variances

The identification of the presence of previously identified variances in cells of an individual, usually a particular patient, can be performed by a number of different techniques as indicated in the Summary above. Such methods include methods utilizing a probe which specifically recognizes the presence of a particular nucleic acid or amino acid sequence in a sample. Common types of probes include nucleic acid hybridization probes and antibodies, for example, monoclonal antibodies, which can differentially bind to nucleic acid sequences differing in one or more variance sites or to polypeptides which differ in one or more amino acid residues as a result of the nucleic acid sequence variance or variances. Generation and use of such probes is well-known in the art and so is not described in detail herein.

Preferably, however, the presence or absence of a variance is determined using nucleotide sequencing of a short sequence spanning a previously identified variance site. This will utilize validated genotyping assays for the polymorphisms previously identified. Since both normal and tumor cell genotypes can be measured, and since tumor material will frequently only be available as paraffin embedded sections (from which RNA cannot be isolated), it will be necessary to utilize genotyping assays that will work on genomic DNA. Thus PCR reactions will be designed, optimized, and validated to accommodate the intron-exon structure of each of the genes. If the gene structure has been published (as it has for some of the listed genes), PCR primers can be designed directly. However, if the gene structure is unknown, the PCR primers may need to be moved around in order to both span the variance and avoid exon-intron boundaries. In some cases one-sided PCR methods such as bubble PCR (Ausubel et al. 1997) may be useful to obtain flanking intronic DNA for sequence analysis.

Using such amplification procedures, the standard method used to genotype normal and tumor tissues will be DNA sequencing. PCR fragments encompassing the variances will be cycle sequenced on ABI 377 automated sequencers using Big Dye chemistry

### C. Correlation of the Presence or Absence of Specific Variances with Differential Treatment Response

Prior to establishment of a diagnostic test for use in the selection of a treatment method or elimination of a treatment method, the presence or absence of one or more specific variances in a gene or in multiple genes is correlated with a differential treatment response. (As discussed above, usually the existence of a variable response and the correlation of such a response to a particular gene is performed first.) Such a differential response can be determined using prospective and/or retrospective data. Thus, in some cases, published reports will indicate that the course of treatment will vary depending on the presence or absence of particular variances. That information can be utilized to create a diagnostic test and/or incorporated in a treatment method as an efficacy or safety determination step.

Usually, however, the effect of one or more variances is separately determined. The determination can be performed by analyzing the presence or absence of particular variances in patients who have previously been treated with a particular treatment method, and correlating the variance presence or absence with the observed course, outcome, and/or development of adverse events in those patents. This approach is useful in cases in which observation of treatment effects was clearly recorded and cell samples are available or can be obtained. Alternatively, the analysis can be performed prospectively, where the presence or absence of the variance or variances in an individual is determined and the course, outcome, and/or development of adverse events in those patients is subsequently or concurrently observed and then correlated with the variance determination.

### Analysis of Haplotypes Increases Power of Genetic Analysis

In some cases, variation in activity due to a single gene or a single genetic variance in a single gene may not be sufficient to account for a clinically significant fraction of the observed variation in patient response to a treatment, e.g., a drug, there may be other factors that account for some of the variation in patient response. Drug response phenotypes may vary continuously, and such (quantitative) traits may be influenced by a number of genes (Falconer and Mackay, Quantitative Genetics, 1997). Although it is impossible to determine a priori the number of genes influencing a quantitative trait, potentially only one or a few loci have large effects, where a large effect is 5-20% of total variation in the phenotype (Mackay, 1995).

Having identified genetic variation in enzymes that may affect action of a specific drug, it is useful to efficiently address its relation to phenotypic variation. The sequential testing for correlation between phenotypes of interest and single nucleotide polymorphisms may be adequate to detect associations if there are major effects associated with single nucleotide changes; certainly it is useful to this type of analysis. However there is no way to know in advance whether there are major phenotypic effects associated with single nucleotide changes and, even if there ale, there is no way to be sure that the salient variance has been identified by screening cDNAs. A more powerful way to address the question of genotype-phenotype correlation is to assort genotypes into haplotypes. (A haplotype is the cis arrangement of polymorphic nucleotides on a particular chromosome.) Haplotype analysis has several advantages compared to the serial analysis of individual polymorphisms at a locus with multiple polymorphic sites.

(1) Of all the possible haplotypes at a locus (2n haplotypes are theoretically possible at a locus with n binary polymorphic sites) only a small fraction will generally occur at a significant frequency in human populations. Thus, association studies of haplotypes and phenotypes will involve testing fewer hypotheses. As a result there is a smaller probability of Type I errors, that is, false inferences that a particular variant is associated with a given phenotype.

(2) The biological effect of each variance at a locus may be different both in magnitude and direction. For example, a polymorphism in the 5′ UTR may affect translational efficiency, a coding sequence polymorphism may affect protein activity, a polymorphism in the 3′ UTR may affect mRNA folding and half life, and so on. Further, there may be interactions between variances: two neighboring polymorphic amino acids in the same domain—say cys/arg at residue 29 and met/val at residue 166—may, when combined in one sequence, for example, 29cys-166val, have a deleterious effect, whereas 29cys-166met, 29arg-166met and 29arg-166val proteins may be nearly equal in activity. Haplotype analysis is the best method for assessing the interaction of variances at a locus.

(3) Templeton and colleagues have developed powerful methods for assorting haplotypes and analyzing haplotype/pherotype associations (Templeton et al., 1987). Alleles which share common ancestry are arranged into a tree structure (cladogram) according to their (inferred) time of origin in a population (that is, according to the principle of parsimony). Haplotypes that are evolutionarily ancient will be at the center of the branching structure and new ones (reflecting recent mutations) will be represented at the periphery, with the links representing intermediate steps in evolution. The cladogram defines which haplotype-phenotype association tests should be performed to most efficiently exploit the available degrees of freedom, focusing attention on those comparisons most likely to define functionally different haplotypes (Haviland et al., 1995). This type of analysis has been used to define interactions between heart disease and the apolipoprotein gene cluster (Haviland et al 1995) and Alzheimer's Disease and the Apo-E locus (Templeton 1995) among other studies, using populations as small as 50 to 100 individuals. The methods of Templeton have also been applied to meaure the genetic determinants of variation in the angiotensin-I converting enzyme gene. (Keavney, B., McKenzie, C. A., Connoll, J. M. C., et al. Measured haplotype analysis of the angiotensin-I converting enzyme gene. Human Molecular Genetics 7: 1745-1751.)

### Methods for Determining Haplotyipes

The goal of haplotyping is to identify the common haplotypes at selected loci that have multiple sites of variance. Haplotype are usually determined at the cDNA level. Several general approaches to identification of haplotyes can be employed. Haplotypes may also be estimated using computational methods or determined definitively using experimental approaches. Computational approaches generally include an expectation maximization (E-M) algorithm (see, for example: Excoffier and Slatkin, Mol. Biol. Evol. 1995) or a combination of Parsimony (see below) and E-M methods.

Haplotypes can be determined experimentally without requirement of a haplotyping method by genotyping samples from a set of pedigrees and observing the segregation of haplotypes. For example families collected by the Centre d'Etude du Polymorphisme Humaine (CEPH) can be used. Cell lines from these families are available from the Coriell Repository. This approach will be useful for cataloging common haplotypes and for validating methods on samples with known haplotypes. The set of haplotypes determined by pedigree analysis can be useful in computational methods, including those utilizing the E-M algorithm.

Haplotypes can also be determined directly from cDNA using the T4E7 procedure. T4E7 cleaves mismatched heteroduplex DNA at the site of the mismatch. If a heteroduplex contains only one mismatch, cleavage will result in the generation of two fragments. However, if a single heteroduplex (allele) contains two mismatches, cleavage will occur at two different sites resulting in the generation of three fragments. The appearance of a fragment whose size corresponds to the distance between the two cleavage sites is diagnostic of the two mismatches being present on the same strand (allele). Thus, T4E7 can be used to determine haplotypes in diploid cells.

An alternative method, allele specific PCR, may be used for haplotyping. The utility of allele specific PCR for haplotyping has already been established (Michalatos-Beloin et al., 1996; Chang et al. 1997). Opposing PCR primers are designed to cover two sites of variance (either adjacent sites or sites spanning one or more internal variances). Two versions of each primer are synthesized, identical to each other except for the 3′ terminal nucleotide. The 3′ terminal nucleotide is designed so that it will hybridize to one but not the other variant base. PCR amplification is then attempted with all four possible primer combinations in separate wells. Because Taq polymerase is very inefficient at extending 3′ mismatches, the only samples which will be amplified will be the ones in which the two primers are perfectly matched for sequences on the same strand (allele). The presence or absence of PCR product allows haplotyping of diploid cell lines. At most two of four possible reactions should yield products. This procedure has been successfully applied, for example, to haplotype the DPD amino acid polymorphisms.

Parsimony methods are also useful for classifying DNA sequences, haplotypes or phenotypic characters. Parsimony principle maintains that the best explanation for the observed differences among sequences, phenotypes (individuals, species) etc., is provided by the smallest number of evolutionary changes. Alternatively, simpler hypotheses are preferable to explain a set of data or patterns, than more complicated ones, and ad hoc hypotheses should be avoided whenever possible (Molecular Systematics, Hillis et al., 1996). Parsimony methods thus operate by minimizing the number of evolutionary steps or mutations (changes from one sequence/character) required to account for a given set of data.

For example, supposing we want to obtain relationships among a set of sequences and construct a structure (tree/topology), we first count the minimum number of mutations that are required for explaining the observed evolutionary changes among a set of sequences. A structure (topology) is constructed based on this number. When once this number is obtained, another structure is tried. This process is continued for all reasonable number of structures. Finally, the structure that required the smallest number of mutational steps is chosen as the likely structure/evolutionary tree for the sequences studied.

### D. Selection of Treatment Method Using Variance Information

1. General

Once the presence or absence of a variance or variances in a gene or genes is shown to correlate with the efficacy or safety of a treatment method, that information can be used to select an appropriate treatment method for a particular patient. In the case of a treatment which is more likely to be effective when administered to a patient who has at least one copy of a gene with a particular variance or variances (in some cases the correlation with effective treatment is for patients who are homozygous for a variance or set of variances in a gene) than in patients with a different variance or set of variances, a method of treatment is selected (and/or a method of administration) which correlates positively with the particular variance presence or absence which provides the indication of effectiveness. As indicated in the Summary, such selection can involve a variety of different choices, and the correlation can involve a variety of different types of treatments, or choices of methods of treatment. In some cases, the selection may include choices between treatments or methods of administration where more than one method is likely to be effective, or where there is a range of expected effectiveness or different expected levels of contra-indication or deleterious effects. In such cases the selection is preferably performed to select a treatment which will be as effective or more effective than other methods, while having a comparatively low level of deleterious effects. Similarly, where the selection is between method with differing levels of deleterious effects, preferably a method is selected which has low such effects but which is expected to be effective in the patient.

Alternatively, in cases where the presence or absence of the particular variance or variances is indicative that a treatment or method of administration is more likely to be ineffective or contra-indicated in a patient with that variance or variances, then such treatment or method of administration is generally eliminated for use in that patient.

2. Diagnostic Methods

Once a correlation between the presence and absence of at least one variance in a gene or genes and an indication of the effectiveness of a treatment, the determination of the presence or absence of that at least one variance provides diagnostic methods, which can be used as indicated in the Summary above to select methods of treatment, methods of administration of a treatment, methods of selecting a patient or patients for a treatment and others aspects in which the determination of the presence or absence of those variances provides useful information for selecting or designing or preparing methods or materials for medical use in the aspects of this invention. As previously stated, such variance determination or diagnostic methods can be performed in various ways as understood by those skilled in the art.

In certain variance determination methods, it is necessary or advantageous to amplify one or more nucleotide sequences in one or more of the genes identified herein. Such amplification can be performed by conventional methods, e.g., using polymerase chain reaction (PCR) amplification. Such amplification methods are well-known to those skilled in the art and will not be specifically described herein. For most applications relevant to the present invention, a sequence to be amplified includes at least one variance site, which is preferably a site or sites which provide variance information indicative of the effectiveness of a method of treatment or method of administration of a treatment, or effectiveness of a second method of treatment which reduces a deleterious effect of a first treatment method, or which enhances the effectiveness of a first method of treatment. Thus, for PCR, such amplification generally utilizes primer oligonucleotides which bind to or extent through at least one such variance site under amplification conditions.

For convenient use of the amplified sequence, e.g., for sequencing, it is beneficial that the amplified sequence be of limited length, but still long enough to allow convenient and specific amplification. Thus, preferably the amplified sequence has a length as described in the Summary.

Also, in certain variance determination, it is useful to sequence one or more portions of a gene or genes, in particular, portions of the genes identified in this disclosure. As understood by persons familiar with nucleic acid sequencing, there are a variety of effective methods. In particular, sequencing can utilize dye termination methods and mass spectrometric methods. The sequencing generally involves a nucleic acid sequence which includes a variance site as indicated above in connection with amplification. Such sequencing can directly provide determination of the presence or absence of a particular variance or set of variances, e.g., a haplotype, by inspection of the sequence (visually or by computer). Such sequencing is generally conducted on PCR amplified sequences in order to provide sufficient signal for practical or reliable sequence determination.

Likewise, in certain variance determinations, it is useful to utilize a probe or probes. As previously described, such probes can be of a variety of different types.

The invention described herein features methods for determining the appropriate identification of a patient diagnosed with a disease or dysfunction based on an analysis of the patient's allele status for a gene listed in U.S. patent application Ser. No. xxxxx. Specifically, the presence of at least one allele indicates that a patient will respond to a candidate therapeutic intervention aimed at treating a neurological clinical symptoms. In a preferred approach, the patient's allele status is rapidly diagnosed using a sensitive PCR assay and a treatment protocol is rendered. The invention also provides a method for forecasting patient outcome and the suitability of the patient for entering a clinical drug trial for the testing of a candidate therapeutic intervention for a neurological disease, condition, or dysfunction.

The findings described herein indicate the predictive value of the target allele in identifying patients at risk for neurologic disease or neurologic dysfunction. In addition, because the underlying mechanism influenced by the allele status is not disease-specific, the allele status is suitable for making patient predictions for diseases not affected by the pathway as well.

The following examples, which describe exemplary techniques and experimental results, are provided for the purpose of illustrating the invention, and should not be construed as limiting.

### EXAMPLE 1

Method for Detecting Variances by Single Strand Conformation Polymorphism (SSCP) Analysis

This example describes the SSCP technique for identification of sequence variances of genes. SSCP is usually paired with a DNA sequencing method, since the SSCP method does not provide the nucleotide identity of variances. One useful sequencing method, for example, is DNA cycle sequencing of 32P labeled PCR products using the Femtomole DNA cycle sequencing kit from Promega (WI) and the instructions provided with the kit. Fragments are selected for DNA sequencing based on their behavior in the SSCP assay.

Single strand conformation polymorphism screening is a widely used technique for identifying an discriminating DNA fragments which differ from each other by as little as a single nucleotide. As originally developed by Orita et al. (Detection of polymorphisms of human DNA by gel electrophoresis as single-strand conformation polymorphisms. Proc Natl Acad Sci U S A. 86(8):2766-70, 1989), the technique was used on genomic DNA, however the same group showed that the technique works very well on PCR amplified DNA as well. In the last 10 years the technique has been used in hundreds of published papers, and modifications of the technique have been described in dozens of papers. The enduring popularity of the technique is due to (1) a high degree of sensitivity to single base differences (>90%) (2) a high degree of selectivity, measured as a low frequency of false positives, and (3) technical ease. SSCP is almost always used together with DNA sequencing because SSCP does not directly provide the sequence basis of differential fragment mobility. The basic steps of the SSCP procedure are described below.

When the intent of SSCP screening is to identify a large number of gene variances it is useful to screen a relatively large number of individuals of different racial, ethnic and/or geographic origins. For example, 32 or 48 or 96 individuals is a convenient number to screen because gel electrophoresis apparatus are available with 96 wells (Applied Biosystems Division of Perkin Elmer Corporation), allowing 3×32, 2×48 or 96 samples to be loaded per gel.

The 32 (or more) individuals screened should be representative of most of the worlds major populations. For example, an equal distribution of Africans, Europeans and Asians constitutes a reasonable screening set. One useful source of cell lines from different populations is the Coriell Cell Repository (Camden, N.J.), which sells EBV immortalized lyphoblastoid cells obtained from several thousand subjects, and includes the racial/ethnic/geographic background of cell line donors in its catalog. Alternatively, a panel of cDNAs can be isolated from any specific target population.

SSCP can be used to analyze cDNAs or genomic DNAs. For many genes cDNA analysis is preferable because for many genes the full genomic sequence of the target gene is not available, however, this circumstance will change over the next few years. To produce CDNA requires RNA. Therefore each cell lines is grown to mass culture and RNA is isolated using an acid/phenol protocol, sold in kit form as Trizol by Life Technologies (Gaithersberg, MD). The unfractionated RNA is used to produce cDNA by the action of a modified Maloney Murine Leukemia Virus Reverse Transcriptase, purchased in kit form from Life Technologies (Superscript II kit). The reverse transcriptase is primed with random hexamer primers to initiate CDNA synthesis along the whole length of the RNAs. This proved useful later in obtaining good PCR products from the 5′ ends of some genes. Alternatively, oligodT can be used to prime CDNA synthesis.

Material for SSCP analysis can be prepared by PCR amplification of the cDNA in the presence of one a 32P labeled dNTP (usually a 32P dCTP). Usually the concentration of nonradioactive dCTP is dropped from 200 uM (the standard concentration for each of the four dNTPs) to about 100 uM, and 32P dCTP is added to a concentration of about 0.1-0.3 uM. This involves adding a 0.3-1 ul (3-10 uCi) of 32P cCTP to a 10 ul PCR reaction. Radioactive nucleotides can be purchased from DuPont/New England Nuclear.

The customary practice is to amplify about 200 base pair PCR products for SSCP, however, an alternative approach is to amplify about 0.8-1.4 kb fragments and then use several cocktails of restriction endonucleases to digest those into smaller fragments of about 0.1-0.4 kb, aiming to have as many fragments as possible between 0.15 and 0.3 kb. The digestion strategy has the advantage that less PCR is required, reducing both time and costs. Also, several different restriction enzyme digests can be performed on each set of samples (for example 96 cDNAs), and then each of the digests can be run separately on SSCP gels. This redundant method (where each nucleotide is surveyed in three different fragments) is reduces both the false negative and false positive rates. For example: a site of variance might lie within 2 bases of the end of a fragment in one digest, and as a result not affect the conformation of that strand; the same variance, in a second or third digest, would likely lie in a location more prone to affect strand folding, and therefore be detected by SSCP.

After digestion, the radiolabelled PCR products are diluted 1:5 by adding formamide load buffer (80% formamide, 1×SSCP gel buffer) and then denatured by heating to 90% C for 10 minutes, and then allowed to renature by quickly chilling on ice. This procedure (both the dilution and the quick chilling) promotes intra- (rather than inter-) strand association and secondary structure formation. The secondary structure of the single strands influences their mobility on nondenaturing gels, presumably by influencing the number of collisions between the molecule and the gel matrix (i.e., gel sieving). Even single base differences consistently produce changes in intrastrand folding sufficient to register as mobility differences on SSCP.

The single strands were then resolved on two gels, one a 5.5% acrylamide, 0.5×TBE gel, the other an 8% acrylamide, 10% glycerol, 1×TTE gel. (Other gel recipes are known to those skilled in the art.) The use of two gels provides a greater opportunity to recognize mobility differences. Both glycerol and acrylamide concentration have been shown to influence SSCP performance. By routinely analyzing three different digests under two gel conditions (effectively 6 conditions), and by looking at both strands under all 6 conditions, one can achieve a 12-fold sampling of each base pair of cDNA. However, if the goal is to rapidly survey many genes or cDNAs then a less redundant procedure would be optimal.

### EXAMPLE 2

Method for Detecting Variances by T4 Endonuclease VII (T4E7) Mismatch Cleavage Method

The enzyme T4 endonuclease VII is derived from the bacteriophage T4. T4 endonuclease VII is used by the bacteriophage to cleave branched DNA intermediates which form during replication so the DNA can be processed and packaged. T4 endonuclease can also recognize and cleave heteroduplex DNA containing single base mismatches as well as deletions and insertions. This activity of the T4 endonuclease VII enzyme can be exploited to detect sequence variances present in the general population.

The following are the major steps involved in identifying sequence variations in a candidate gene by T4 endonuclease VII mismatch cleavage:

1. Amplification by the polymerase chain reaction (PCR) of 400-600 bp regions of the candidate gene from a panel of DNA samples The DNA samples can either be CDNA or genomic DNA and will represent some cross section of the world population.

2. Mixing of a fluorescently labeled probe DNA with the sample DNA. Heating and cooling the mixtures causing heteroduplex formation between the probe DNA and the sample DNA.

3. Addition of T4 endonuclease VII to the heteroduplex DNA samples. T4 endonuclease will recognize and cleave at sequence variance mismatches formed in the heteroduplex DNA.

4. Electrophoresis of the cleaved fragments on an ABI sequencer to determine the site of cleavage.

5. Sequencing of a subset of PCR fragments identified by T4 endonuclease VI to contain variances to establish the specific base variation at that location.

A more detailed description of the procedure is as follows:

A candidate gene sequence is downloaded from an appropriate database. Primers for PCR amplification are designed which will result in the target sequence being divided into amplification products of between 400 and 600 bp. There will be a minimum of a 50 bp of overlap not including the primer sequences between the 5′ and 3′ ends of adjacent fragments to ensure the detection of variances which are located close to one of the primers.

Optimal PCR conditions for each of the primer pairs is determined experimentally. Parameters including but not limited to annealing temperature, pH, MgCl2 concentration, and KCl concentration will be varied until conditions for optimal PCR amplification are established. The PCR conditions derived for each primer pair is then used to amplify a panel of DNA samples (CDNA or genomic DNA) which is chosen to best represent the various ethnic backgrounds of the world population or some designated subset of that population.

One of the DNA samples is chosen to be used as a probe. The same PCR conditions used to amplify the panel are used to amplify the probe DNA. However, a flourescently labeled nucleotide is included in the deoxy-nucleotide mix so that a percentage of the incorporated nucleotides will be fluorescently labeled.

The labeled probe is mixed with the corresponding PCR products from each of the DNA samples and then heated and cooled rapidly. This allows the formation of heteroduplexes between the probe and the PCR fragments from each of the DNA samples. T4 endonuclease VII is added directly to these reactions and allowed to incubate for 30 min. at 37 C. 10 ul of the Formamide loading buffer is added directly to each of the samples and then denatured by heating and cooling. A portion of each of these samples is electrophoresed on an ABI 377 sequencer. If there is a sequence variance between the probe DNA and the sample DNA a mismatch will be present in the heteroduplex fragment formed. The enzyme T4 endonuclease VII will recognize the mismatch and cleave at the site of the mismatch. This will result in the appearance of two peaks corresponding to the two cleavage products when run on the ABI 377 sequencer.

Fragments identified as containing sequencing variances are subsequently sequenced using conventional methods to establish the exact location and sequence variance.

### EXAMPLE 3

Method for Detecting Variances by DNA sequencing.

Sequencing by the Sanger dideoxy method or the Maxim Gilbert chemical cleavage method is widely used to determine the nucleotide sequence of genes. Presently, a worldwide effort is being put forward to sequence the entire human genome. The Human Genome Project as it is called has already resulted in the identification and sequencing of many new human genes. Sequencing can not only be used to identify new genes, but can also be used to identify variations between individuals in the sequence of those genes.

The following are the major steps involved in identifying sequence variations in a candidate gene by sequencing:

1. Amplification by the polymerase chain reaction (PCR) of 400-700 bp regions of the candidate gene from a panel of DNA samples The DNA samples can either be cDNA or genomic DNA and will represent some cross section of the world population.

2. Sequencing of the resulting PCR fragments using the Sanger dideoxy method. Sequencing reactions are performed using flourescently labeled dideoxy terminators and fragments are separated by electrophoresis on an ABI 377 sequencer or its equivalent.

3. Analysis of the resulting data from the ABI 377 sequencer using software programs designed to identify sequence variations between the different samples analyzed.

A more detailed description of the procedure is as follows:

A candidate gene sequence is downloaded from an appropriate database. Primers for PCR amplification are designed which will result in the target sequence being divided into amplification products of between 400 and 700 bp. There will be a minimum of a 50 bp of overlap not including the primer sequences between the 5′ and 3′ ends of adjacent fragments to ensure the detection of variances which are located close to one of the primers.

Optimal PCR conditions for each of the primer pairs is determined experimentally. Parameters including but not limited to annealing temperature, pH, MgCl2 concentration, and KCl concentration will be varied until conditions for optimal PCR amplification are established. The PCR conditions derived for each primer pair is then used to amplify a panel of DNA samples (cDNA or genomic DNA) which is chosen to best represent the various ethnic backgrounds of the world population or some designated subset of that population.

PCR reactions are purified using the QIAquick 8 PCR purification kit (Qiagen cat# 28142) to remove nucleotides, proteins and buffers. The PCR reactions are mixed with 5 volumes of Buffer PB and applied to the wells of the QIAquick strips. The liquid is pulled through the strips by applying a vacuum. The wells are then washed two times with 1 ml of buffer PE and allowed to dry for 5 minutes under vacuum. The PCR products are eluted from the strips using 60 ul of elution buffer.

The purified PCR fragments are sequenced in both directions using the Perkin Elmer ABI PrismTM Big DyeTM terminator Cycle Sequencing Ready Reaction Kit (Cat# 4303150). The following sequencing reaction is set up: 8.0 ul Terminator Ready Reaction Mix, 6.0 ul of purified PCR fragment, 20 picomoles of primer, deionized water to 20 ul. The reactions are run through the following cycles 25 times: 96oC for 10 second, annealing temperature for that particular PCR product for 5 seconds, 60oC for 4 minutes.

The above sequencing reactions are ethanol precipitated directly in the PCR plate, washed with 70% ethanol, and brought up in a volume of 6 ul of formamide dye. The reactions are heated to 90 oC for 2 minutes and then quickly cooled to 4 oC. 1 ul of each sequencing reaction is then loaded and run on an ABI 377 sequencer.

The output for the ABI sequencer appears as a series of peaks where each of the different nucleotides, A, C, G, and T appear as a different color. The nucleotide at each position in the sequence is determined by the most prominent peak at each location. Comparison of each of the sequencing outputs for each sample can be examined using software programs to determine the presence of a variance in the sequence. One example of heterozygote detection using sequencing with dye labeled terminators is described by Kwok et. al. (Kwok, P.-Y.; Carlson, C.; Yager, T. D., Ankener, W.,and D. A. Nickerson, Genomics 23, 138-144, 1994). The software compares each of the normalized peaks between all the samples base by base and looks for a 40% decrease in peak height and the concomitant appearance of a new peak underneath. Possible variances flagged by the software are further analyzed visually to confirm their validity.

### EXAMPLE 4

Hardy-Weinberg Equilibrium

Evolution is the process of change and diversification of organisms through time, and evolutionary change affects morphology, physiology and reproduction of organisms, including humans. These evolutionary changes are the result of changes in the underlying genetic or hereditary material. Evolutionary changes in a group of interbreeding individuals or Mendelian population, or simply populations, are described in terms of changes in the frequency of genotypes and their constituent alleles. Genotype frequencies for any given generation is the result of the mating among members (genotypes) of their previous generation. Thus, the expected proportion of genotypes from a random union of individuals in a given population is essential for describing the total genetic variation for a population of any species. For example, the expected number of genotypes that could form from the random union of two alleles, A and a, of a gene are AA, Aa and aa. The expected frequency of genotypes in a large, random mating population was discovered to remain constant from generation to generation; or achieve Hardy-Weinberg equilibrium, named after its discoverers. The expected genotypic frequencies of alleles A and a (AA, 2Aa, aa) are conventionally described in terms of p2+2pq+q2 in which p and q are the allele frequencies of A and a. In this equation (p²+2pq+q²=1), p is defined as the frequency of one allele and q as the frequency of another allele for a trait controlled by a pair of alleles (A and a). In other words, p equals all of the alleles in individuals who are homozygous dominant (AA) and half of the alleles in individuals who are heterozygous (Aa) for this trait. In mathematical terms, this is

p=AA+½Aa

Likewise, q equals the other half of the alleles for the trait in the population, or

q=aa+½Aa

Because there are only two alleles in this case, the frequency of one plus the frequency of the other must equal 100%, which is to say

p+q=1

Alternatively,

p=1−q OR q=1−p

All possible combinations of two alleles can be expressed as:

(p+q)²−1

or more simply,

p²+2pq+q²=1

In this equation, if p is assumed to be dominant, then p² is the frequency of homozygous dominant (AA) individuals in a population, 2pq is the frequency of heterozygous (Aa) individuals, and q² is the frequency of homozygous recessive (aa) individuals.

From observations of phenotypes, it is usually only possible to know the frequency of homozygous dominant or recessive individuals, because both dominant and recessives will express the distinguishable traits. However, the Hardy-Weinberg equation allows us to determine the expected frequencies of all the genotypes, if only p or q is known. Knowing p and q, it is a simple matter to plug these values into the Hardy-Weinberg equation (p²+2pq+q²=1). This then provides the frequencies of all three genotypes for the selected trait within the population.

This illustration shows Hardy-Weinberg frequency distributions for the genotypes AA, Aa, and aa at all values for frequencies of the alleles, p and q. It should be noted that the proportion of heterozygotes increases as the values of p and q approach 0.5.

### Linkage Disequilibirum

Linkage is the tendency of genes or DNA sequences (e.g. SNPs) to be inherited together as a consequence of their physical proximity on a single chromosome. The closer together the markers are, the lower the probability that they will be separated during DNA crossing over, and hence the greater the probability that they will be inherited together. Suppose a mutational event introduces a “new” allele in the close proximity of a gene or an allele. The new allele will tend to be inherited together with the alleles present on the “ancestral,” chromosome or haplotype. However, the resulting association, called linkage disequilibrium, will decline over time due to recombination. Linkage disequilibrium has been used to map disease genes. In general, both allele and haplotype frequencies differ among populations. Linkage disequilibrium is varied among the populations, being absent in some and highly significant in others.

### Quantification of the Relative Risk of Observable Outcomes of a Pharmacogenetics Trial

Let PlaR be the placebo response rate (0%(PlaR(100%) and TntR be the treatment response rate (0%(TntR(100%) of a classical clinical trial. ObsRR is defined as the relative risk between TntR and PlaR:

ObsRR=TntR/PlaR.

Suppose that in the treatment group there is a polymorphism in relation to drug metabolism such as the treatment response rate is different for each genotypic subgroup of patients. Let q be the allele a frequency of a recessive biallelic locus (e.g. SNP) and p=1−q the allele A frequency. Following Hardy-Weinberg equilibrium, the relative frequency of homozygous and heterozygous patients are as follows:

AA: p2 Aa: 2pq aa: q²

with (p2+2pq+q2)=1.

Defining AAR, AaR, aaR as respectively the response rates of the AA, Aa and aa patients, we have the following relationship:

TntR=AAR*p2+AaR*2pq+aaR*q2.

Suppose that the aa genotypic group of patients has the lowest response rate, i.e. a response rate equal to the placebo response rate (which means that the polymorphism has no impact on natural disease evolution but only on drug action) and let's define ExpRR as the relative risk between AAR and aaR, as

ExpRR=AAR/aaR.

From the previous equations, we have the following relationships:

ObsRR(ExpRR(1/PlaR

TntR/PlaR=(AAR*p2+AaR*2pq+aaR*q2)/PlaR

The maximum of the expected relative risk, max(ExpRR), corresponding to the case of heterozygous patients having the same response rate as the placebo rate, is such that:

ObsRR=ExpRR*p2+2pq+q2 [symbol] ExpRR=(ObsRR−2pq−q2)/p2

The minimum of the expected relative risk, min(ExpRR), corresponding to the case of heterozygous patients having the same response rate as the homozygous non-affected patients, is such that:

ObsRR=ExpRR*(p2+2pq)+q2 [symbol] ExpRR=(ObsRR−q2)/(p2+2pq)

For example, if q=0.4, PlaR=40% and ObsRR=1.5 (i.e. TntR=60%), then 1.6 (ExpRR(2.4. This means that the best treatment response rate we can expect in a genotypic subgroup of patients in these conditions would be 95.6% instead of 60%.

This can also be expressed in terms of maximum potential gain between the observed difference in response rates (TntR−PlaR) without any pharmacogenetic hypothesis and the maximum expected difference in response rates (max(ExpRR)*PlaR−TntR) with a strong pharmacogenetic hypothesis:

(max(ExpRR)*PlaR−TntR)=[(ObsRR−2pq−q2)/p2]*PlaR−TntR

[symbol]

(max(ExpRR)*PlaR−TntR)=[TntR−PlaR*(2pq+q2)−TntR*p2]/p2

[symbol]

(max(ExpRR)*PlaR−TntR)=[TntR*(1−p2)−PlaR*(2pq+q2)]/p2

[symbol]

(max(ExpRR)*PlaR−TntR)=[(1−p2)/p2]*(TntR−PlaR)

that is for the previous example,

(95.6%−60%)=[(1−0.62)/0.62]*(60%−40%)=35.6%

Suppose that, instead of one SNP, we have p loci of SNPs for one gene. This means that we have 2p possible haplotypes for this gene and (2p)(2p−1)/2 possible genotypes. And with 2 genes with p1 and p2 SNP loci, we have [(2p1)(2p1−1)/2]*[(2p2)(2p2−1)/2] possibilities; and so on. Examining haplotypes instead of combinations of SNPs is especially useful when there is linkage disequilibrium enough to reduce the number of combinations to test, but not complete since in this latest case one SNP would be sufficient. Yet the problem of frequency above still remains with haplotypes instead of SNPs since the frequency of a haplotype cannot be higher than the highest SNP frequency involved.

Statistical Methods to be used in Objective Analyses The statistical significance of the differences between variance frequencies can be assessed by a Pearson chi-squared test of homogeneity of proportions with n-1 degrees of freedom. Then, in order to determine which variance(s) is(are) responsible for an eventual significance, we can consider each variance individually against the rest, up to n comparisons, each based on a 2×2 table. This should result in chi-squared tests that are individually valid, but taking the most significant of these tests is a form of multiple testing. A Bonferroni's adjustment for multiple testing will thus be made to the P-values, such as p*=1−(1−p)n.

The statistical significance of the difference between genotype frequencies associated to every variance can be assessed by a Pearson chi-squared test of homogeneity of proportions with 2 degrees of freedom, using the same Bonferroni's adjustment as above.

Testing for unequal haplotype frequencies between cases and controls can be considered in the same framework as testing for unequal variance frequencies since a single variance can be considered as a haplotype of a single locus. The relevant likelihood ratio test compares a model where two seqarate sets of haplotype frequencies apply to the cases and controls, to one where the entire sample is characterized by a single common set of haplotype frequencies. This can be performed by repeated use of a computer program (Terwilliger and Ott, 1994, Handbook of Human Linkage Analysis, Baltimore, John Hopkins University Press) to successively obtain the log-likelihood corresponding to the set of haplotpe frequency estimates on the cases (lnLcase), on the controls (lnLcontrol), and on the overall (lnLcombined). The test statistic 2((InLcase)+(InLcontrol)−(InLcombined)) is then chi-squared with r−1 degrees of freedom (where r is the number of haplotypes).

To test for potentially confounding effects or effect-modifiers, such as sex, age, etc., logistic regression can be used with case-control status as the outcome variable, and genotypes and covariates (plus possible interactions) as predictor variables.

### EXAMPLE 5

Exemplary Pharmacogenetic Analysis Steps

In accordance with the discussion of distribution frequencies for variances, alleles, and haplotypes, variance detection, and correlation of variances or haplotypes with treatment response variability, the points below list major items which will typically be performed in an analysis of the pharmacogenetic determination of the effects of variances in the treatment of a disease and the selection/optimization of treatment.

1) List candidate gene/genes for a known genetic disease, and assign them to the respective metabolic pathways.

2) Determine their alleles, observed and expected frequencies, and their relative distributions among various ethnic groups, gender, both in the control and in the study (case) groups.

3) Measure the relevant clinical/phenotypic (biochemical/physiological) variables of the disease.

4) If the causal variance/allele in the candidate gene is unknown, then determine linkage disequilibria among variances of the candidate gene(s).

5) Divide the regions of the candidate genes into regions of high linkage disequilibrium and low disequilibrium.

6) Develop haplotypes among variances that show strong linkage disequilibrium using the computation methods.

7) Determine the presence of rare haplotypes experimentally. Confirm if the computationally determined rare haplotypes agree with the experimentally determined haplotypes.

8) If there is a disagreement between the experimentally determined haplotypes and the computationally derived haplotypes, drop the computationally derived rare haplotypes, construct cladograms from these haplotypes using the Templeton (1987) algorithm.

9) Note regions of high recombination. Divide regions of high recombination further to see patterns of linkage disequilibria.

10) Establish association between cladograms and clinical variables using the nested analysis of variance as presented by Templeton (1995), and assign causal variance to a specific haplotype.

11) For variances in the regions of high recombination, use permutation tests for establishing associations between variances and the phenotypic variables.

12) If two or more genes are found to affect a clinical variable determine the relative contribution of each of the genes or variances in relation to the clinical variable, using step-wise regression or discriminant function or principal component analysis.

13) Determine the relative magnitudes of the effects of any of the two variances on the clinical variable due to their genetic (additive, dominant or epistasis) interaction.

14) Using the frequency of an allele or haplotypes, as well as biochemical/clinical variables determined in the in vitro or in vivo studies, determine the effect of that gene or allele on the expression of the clinical variable, according to the measured genotype approach of Boerwinkle et al (Ann. Hum. Genet 1986).

15) Stratify ethnic/clinical populations based on the presence or absence of a given allele or a haplotype.

16) Optimize drug dosages based on the frequency of alleles and haplotypes as well as their effects using the measured genotype approach as a guide.

### EXAMPLE 6

Exemplary Pharmacogenetic Analysis Steps—biological function analysis

In many cases when a gene which may affect drug action is found to exhibit variances in the gene, RNA, or protein sequence, it is preferable to perform biological experiments to determine the biological impact of the variances on the structure and function of the gene or its expressed product and on drug action. Such experiments may be performed in vitro or in vivo using methods known in the art.

The points below list major items which may typically be performed in an analysis of the effects of variances in the treatment of a disease and the selection/optimization of treatment using biological studies to determine the structure and function of variant forms of a gene or its expressed product.

1) List candidate gene/genes for a known genetic disease, and assign them to the respective metabolic pathways.

2) Identify variances in the gene sequence, the expressed mRNA sequence or expressed protein sequence.

3) Match the position of variances to regions of the gene, mRNA, or protein with known biological functions. For example, specific sequences in the promotor of a gene are known to be responsible for determining the level of expression of the gene; specific sequences in the mRNA are known to be involved in the processing of nuclear mRNA into cytoplasmic mRNA including splicing and polyadenylation; and certain sequences in proteins are known to direct the trafficking of proteins to specific locations within a cell and to constitute active sites of biological functions including the binding of proteins to other biological consituents or catalytic functions. Variances in sites such as these, and others known in the art, are candidates for biological effects on drug action.

4) Model the effect of the variance on mRNA or protein structure. Computational methods for predicting the structure of mRNA are known and can be used to assess whether a specific variance is likely to cause a substantial change in the structure of mRNA. Computational methods can also be used to predict the structure of peptide sequences enabling predictions to be made concerning the potential impact of the variance on protein function. Most useful are structures of proteins determined by X-ray diffraction, NMR or other methods known in the art which provide the atomic structure of the protein. Computational methods can be used to consider the effect of changing an amino acid within such a structure to determine whether such a change would disrupt the structure and/or function of the protein. Those skilled in the art will recognize that this analysis can be performed on crystal structures of the protein known to have a variance as well as homologous proteins expressed from different loci in the human genome, or homologous proteins from other species, or non-homologous but analogous proteins with similar functions from humans or other species.

5) Produce the gene, mRNA or protein in amounts sufficient to experimentally characterize the structure and function of the gene, mRNA or protein. It will be apparent to those skilled in the art that by comparing the activity of two genes or their products which differ by a single variance, the effect of the variance can be determined. Methods for producing genes or gene products which differ by one or more bases for the purpose of experimental analysis are known in the art.

6) Experimental methods known in the art can be used to determine whether a specific variance alters the transcription of a gene and translation into a gene product. This involves producing amounts of the gene by molecular cloning sufficient for in vitro or in vivo studies. Methods for producing genes and gene products are known in the art and include cloning of segments of genetic material in prokaryotes or eukarotic hosts, run off transcription and cell-free translation assays that can be performed in cell free extracts, transfection of DNA into cultured cells, introduction of genes into live animals or embryos by direct injection or using vehicles for gene delivery including transfection mixtures or viral vectors.

7) Experimental methods known in the art can be used to determine whether a specific variance alters the ability of a gene to be transcribed into RNA. For example, run off transcription assays can be performed in vitro or expression can be characterized in transfected cells or transgenic animals.

8) Experimental methods known in the art can be used to determine whether a specific variance alters the processing, stability, or translation of RNA into protein. For example, reticulocyte lysate assays can be used to study the production of protein in cell free systems, transfection assays can be designed to study the production of protein in cultured cells, and the production of gene products can be measured in transgenic animals.

9) Experimental methods known in the art can be used to determine whether a specific variant alters the activity of an expressed protein product. For example, protein can be producted by reticulocyte lystae systems or by introducing the gene into prokaryotic organisms such as bacteria or lowre eukaryotic organisms such as yeast or fungus), or by introducing the gene into cultured cells or transgenic animals. Protein produced in such systems can be extracted or purified and subjected to bioassays known to those in the art as measures of the action of that particular protein. Bioassays may involve, but are not limited to, binding, inhibition, or catalytic functions.

10) Those skilled in the art will recognize that it is sometimes preferred to perform the above experiments in the presence of a specific drug to determine whether the drug has differential effects on the activity being measured. Alternatively, studies may be performed in the presence of an analogue or metabolite of the drug.

11) Using methods described above, specific variances which alter the biological function of a gene or its gene product that could have an impact on drug action can be identified. Such variances are then studied in clinical trial populations to determine whether the presence or absence of a specific variance correlates with observed clinical outcomes such as efficacy or toxicity.

12) It will be further recognized that there may be more than one variance within a gene that is capable of altering the biological function of the gene or gene product. These variances may exhibit similar, synergistic effects, or may have opposite effects on gene function. In such cases, it is necessary to consider the haplotype of the gene, namely the combination of variances that are present within a single allele, to assess the composite function of the gene or gene product.

13) Perform clinical trials with stratification of patients based on presence or absence of a given variance, allele or haplotype of a gene. Establish associations between observed drug responses such as toxicity, efficacy, drug response, or dose toleration and the presence or absense of a specific variance, allele, or haplotype.

14) Optimize drug dosage or drug usage based on the presence of the variant.

### Other Embodiments

The invention described herein provides a method for identifying patients with a risk of developing neurological disease or dysfunction by determining the patients allele status for a gene listed in U.S. patent application Ser. No. 09/689,506 and providing a forecast of the patients ability to respond to or tolerate a given drug treatment. In particular, the invention provides a method for determining, based on the presence or absence of a polymorphism, a patient's likely response to drug therapies of neurological disease or dysfunction. Given the predictive value of the described polymorphisms a candidate polymorphism is likely to have a similar predictive value for other drugs acting through other pharmacological mechanisms. Thus, the methods of the invention may be used to determine a patient's response to other drugs including, without limitation, antihypertensives, anti-obesity, anti-hyperlipidemic, or anti-proliferative, antioxidants, or enhancers of terminal differentiation.

In addition, while determining the presence or absence of the candidate allele is a clear predictor determining the efficacy of a drug on a given patient, other allelic variants of reduced catalytic activity are envisioned as predicting drug efficacy using the methods described herein. In particular, the methods of the invention may be used to treat patients with any of the possible variances, e.g., as described in Table 3 of Stanton et al., U.S. application Ser. No. 09/300,747.

In addition, while the methods described herein are preferably used for the treatment of human patients, non-human animals (e.g., dogs, cats, sheep, cattle and other bovines, swine, and apes and other non-human primates) may also be treated using the methods of the invention.

It will be readily apparent to one skilled in the art that varying substitutions and modifications may be made to the invention disclosed herein without departing from the scope and spirit of the invention. For example, using other compounds, and/or methods of administration are all within the scope of the present invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The details of one or more embodiments of the invention are set forth in the accompanying drawings and the description below. Other features, objects, and advantages of the invention will be apparent from the description and drawings, and from the claims.

1. A method for identifying phenotypes that vary in cell lines as a result of genetic variation, comprising:
(a) measuring one or more phenotypes in cell lines from one or more pedigrees; and
(b) testing whether the pattern of phenotype data in the cell lines conforms to the rules of Mendelian transmission,
wherein conformation of said phenotype data to the rules of Mendelian transmission is indicative that said phenotype varies in cell lines as a result of genetic variation.

2. A method for identifying phenotypes that vary in cell lines as a result of genetic variation, comprising:
(a) measuring one or more phenotypes in cell lines from one or more pedigrees; and
(b) testing whether the pattern of phenotype variation in the cell lines segregates in the pedigree so as to produce a LOD score of at least 2 with one or more loci, and wherein detection of a LOD score of at least 2 is indicative that said phenotype varies in cell lines as a result of genetic variation.

3. The method of claim 1, wherein the phenotype is the mRNA level of a selected gene.

4. The method of claim 2 where the LOD score is at least 3.

5. The method of any of claims 1 or 2, wherein the cell lines are derived from the CEPH pedigrees.

6. The method of any of claims 1 or 2, wherein the gene or genes responsible for the inter-cell line variation in phenotype are mapped to chromosomal loci by comparison of the pattern of segregation of the phenotype in the cell lines with the pattern of segregation of known mapped variances in the same cell lines.

7. The method of claim 4, wherein one or more candidate genes are evaluated by determining if their chromosomal position is one of the chromosomal positions (loci) that displays segregation with the phenotype.

8. The method of any of claims 1 or 2, wherein at least 15 cell lines from related individuals are tested.

9. The method of any of claims 1 or 2, wherein the cells are subjected to a treatment before measuring the phenotype, the treatment selected from the group consisting of:
a. addition of a compound to the cells,
b. change in the nutritional environment of the cells, and
c. change in the physical environment of the cells.

10. A method for identifying mRNAs that vary in levels as a result of genetic variation, comprising:
a. measuring levels of one or more specific mRNAs in cell lines from one or more pedigrees; and
b. testing whether the mRNA levels of said one or more specific mRNAs in said cell lines conforms to the rules of Mendelian transmission,
wherein conformation of any of said mRNA levels to the rules of Mendelian transmission is indicative that said mRNA level varies in cell lines as a result of genetic variation.

11. The method of claim 10, wherein said cell lines are derived from one or more of the CEPH pedigrees.

12. The method of claim 10, wherein the gene or genes responsible for the intersubject variation in levels of specific mRNAs are mapped to chromosomal loci by comparison of the pattern of segregation of the mRNA levels in the cell lines with the pattern of segregation of variances that are already mapped to the human genome.

13. The method of claim 10, wherein at least 100 cell lines from related individuals are tested.

14. The method of claim 10, wherein said cells are subjected to a treatment before performing the RNA analysis, the treatment selected from the group consisting of:
a. addition of a compound to the cells,
b. change in the nutritional environment of the cells, and
c. change in the physical environment of the cells.

15. A method for the identification of phenotypes that vary among cell lines as a consequence of genetic variation, the method comprising:
a. Determining the genotype of a set of cell lines from unrelated subjects at candidate genes for the phenotypes of interest;
b. measuring the phenotype in the cell lines; and
c. Measuring whether genetic variation among the cell lines correlates with variation in the phenotype.

16. The method of claim 15 where at lest 20 cell lines are analyzed.
